(19) United States
(12) Patent Application Publication
(10) Pub. No.: US 2016/0106711 A1
(43) Pub. Date: Apr. 21, 2016

(54) 3-3-Di-Substituted-Oxindoles as Inhibitors of Translation Initiation

(71) Applicant: President and Fellows of Harvard College, Cambridge, MA (US)

(72) Inventors: José A. HALPERIN, Brookline, MA (US); Michael CHOREV, Chestnut Hill, MA (US); Bertal Huseyin AKTAS, Newton, MA (US)

(21) Appl. No.: 14/429,622

(22) PCT Filed: Sep. 20, 2013
(86) PCT No.: PCT/US2013/060916

§ 371 Date: Mar. 19, 2015

Related U.S. Application Data
(60) Provisional application No. 61/703,373, filed on Sep. 20, 2012.

(51) Int. Cl. A61K 31/404; A61K 31/7056; A61K 31/4439; A61K 31/5377; A61K 31/496

(52) U.S. Cl. CPC A61K 31/404; A61K 31/5377; A61K 31/496; A61K 31/4439; A61K 31/7056

(57) ABSTRACT

Compositions and methods for inhibiting translation are provided. Compositions, methods and kits for treating (1) cellular proliferative disorders, (2) non-proliferative, degenerative disorders, (3) viral infections, and/or (4) disorders associated with viral infections, using diaryloxindole compounds are described.

### RELATED APPLICATION DATA

This application claims priority to U.S. Provisional Application No. 61/703,373, filed on Sep. 20, 2012 which is hereby incorporated herein by reference in its entirety for all purposes.

### STATEMENT OF GOVERNMENT INTERESTS

This invention was made with government support under NIH grant CA087427. The Government has certain rights in the invention.

### FIELD

The present invention relates to novel compounds which inhibit translation initiation, pharmaceutical compositions of the novel compounds, and methods of treating medical disorders.

### BACKGROUND

Translation, the mRNA-directed synthesis of proteins, occurs in three distinct steps: initiation, elongation and termination. Translation initiation is a complex process in which the two ribosomal subunits and methionyl tRNA (met-tRNA) assemble on a properly aligned mRNA to commence chain elongation at the AUG initiation codon. The established scanning mechanism for initiation involves the formation of a ternary complex among eukaryotic initiation factor 2 (eIF2, GTP and met-tRNA. The ternary complex recruits the 40S ribosomal subunit to form the 43S pre-initiation complex. This complex recruits mRNA in cooperation with other initiation factors such as eukaryotic initiation factor 4E (eIF4E), which recognizes the 7-methyl-guanidine cap (m-⁷GTP cap) in an mRNA molecule and forms the 48S pre-initiation complex. Cap recognition facilitates the 43S complex entry at the 5′ end of a capped mRNA. Subsequently, this complex migrates linearly until it reaches the first AUG codon, where a 60S ribosomal subunit joins the complex, and the first peptide bond is formed (Pain (1996) Eur. J. Biochem., 236:747-771).

Several features of the mRNA structure influence the efficiency of its translation. These include the m-⁷GTP cap, the primary sequence surrounding the AUG codon and the length and secondary structure of the 5′ untranslated region (5′ UTR). Indeed, a moderately long, unstructured 5′ UTR with a low G and C base content seems to be optimal to ensure high translational efficiency. Surprisingly, sequence analysis of a large number of vertebrate cDNAs has shown that although most transcripts have features that ensure translational fidelity, many do not appear to be designed for efficient translation (Kozak (1991) J. Cell. Biol., 115:887-903). Many vertebrate mRNAs contain 5′ UTRs that are hundreds of nucleotides long with a remarkably high GC content, indicating that they are highly structured because G and C bases tend to form highly stable bonds. Because highly structured and stable 5′ UTRs are the major barrier to translation, mRNAs with stable secondary structure in their 5′ UTR are translated inefficiently and their translation is highly dependent on the activity of translation initiation factors.

mRNAs with complex, highly structured 5′ UTRs include a disproportionately high number of proto-oncogenes such as the G1 cyclins, transcription and growth factors, cytokines and other critical regulatory proteins. In contrast, mRNAs that encode globins, albumins, histones and other housekeeping proteins rarely have highly structured, GC-rich 5′ UTRs (Kozak (1994) Biochimie, 76; 815-21; Kozak (1999) Gene, 234:187-208). The fact that genes encoding for regulatory but not for housekeeping proteins frequently produce transcripts with highly structured 5′ UTRs indicates that extensive control of the expression of regulatory genes occurs at the level of translation. In other words, low efficiency of translation is a control mechanism which modulates the yield of proteins such as cyclins, mos, c-myc, VEGF, TNF, among others, that could be harmful if overproduced.

Translation initiation is a critical step in the regulation of cell growth because the expression of most oncogenes and cell growth regulatory proteins is translationally regulated. One approach to inhibiting translation initiation has recently been identified using small molecule known as translation initiation inhibitors. Without intending to be bound by theory, FIG. 1 sets forth a summary of the anti-cancer mechanism of action of translation initiation inhibitors such as clotrimazole (CLT) and the diaryloxindole (DAO) compounds of the present invention. CLT inhibits translation initiation by sustained depletion of intracellular Ca²⁺ stores. Depletion of intracellular Ca²⁺ stores activates “interferon-inducible” “double-stranded RNA activated” protein kinase (PKR) which phosphorylates and thereby inhibits the subunit of eIF2 Since the activity of eIF2 is required for translation initiation, its inhibition by compounds such as CLT reduces the overall rate of protein synthesis. Because most cell regulatory proteins are encoded for by mRNAs containing highly structured 5′ UTRs, they are poorly translated and their translation depends heavily on translation initiation factors such as eIF2 and eIF4. Therefore, inhibition of translation initiation preferentially affects the synthesis and expression of growth regulatory proteins such as G1 cyclins. Sequential synthesis and expression of G1 cyclins (D1, E and A) is necessary to drive the cell cycle beyond the restriction point in late G1. Thus, the decreased synthesis and expression of G1 cyclins resulting from CLT-induced inhibition of translation initiation causes cell cycle arrest in G1 and inhibits cancer cell and tumor growth (Aktas et al. (1998) Proc. Natl. Acad. Sci. USA, 95:8280-8285, incorporated herein by reference in its entirety for all purposes).

Like CLT, the n-3 polyunsaturated fatty acid eicosapentaenoic acid (EPA) depletes internal calcium stores, and exhibits anti-carcinogenic activity. Unlike CLT, however, EPA is a ligand of peroxisome proliferator-activated receptor gamma (PPAR), a fatty acid-activated transcription factor. Although EPA and other ligands of PPAR, such as troglitazone and ciglitazone, inhibit cell proliferation, they do so in a PPAR-independent manner (Palakurthi et al. (2000) Cancer Research, 60:2919; and Palakurthi et al. (2001) Cancer Research, 61:6213, incorporated herein by reference in their entirety for all purposes).

Translation initiation is an accepted target for cancer treatments. See Funda Meric and Kelly Hunt, Translation Initiation in Cancer: A Novel Target for Therapy, Molecular Cancer Therapeutics, Vol. 1, 971-979, September 2002; S. J. Watkins and C. J. Norbury, Translation Initiation and Its Deregulation During Tumorigenesis, British Journal of Cancer (2002) 86, 1023-1027; Igor Rosenwald, The Role of Translation in Neoplastic Transformation from a Pathologist's Point of View, Oncogene (2004) 23, 3230-3247; Igor Rosenwald, Songtao Wang, Lou Savas, Bruce Woda, James Pullman, Expression of Translation Initiation Factor eIF-2α is Increased in Benign and Malignant Melanocytic and Colonic Epithelial Neoplasms, Cancer, Vol. 98, No. 5, (2003); Songtao Wang, Igor Rosenwald, Michael Hutzler, German Pihan, Lou Savas, Jane-Jane Chen and Bruce Woda, Expression of the Eukaryotic Translation Initiation Factors 4E and 2α in Non-Hodgkin's Lymphomas, American Journal of Pathology, Vol. 155, 247-255 (1999); B. Bilanges and D. Stokoe, Mechanisms of Translational Deregulation in Human Tumors and Therapeutic Intervention Strategies, Oncogene (2007) 26, 5973-5990; Songtao Wang, Ricardo Lloyd, Michael Hutzler, Igor Rosenwald, Marjorie Safran, Nilima Patwardhan and Ashraf Khan, Expression of Eukaryotic Translation Initiation Factors 4E and 2α Correlates with the Progression of Thyroid Carcinoma, Thyroid, Vol. 11, No. 12 1101-1107 (2001).

### SUMMARY

Embodiments of the present invention are directed to compounds that inhibit translation initiation, and the use of such compounds or combination of compounds for treating (1) cellular proliferative disorders, (2) non-proliferative, degenerative disorders, (3) viral infections, and/or (4) disorders associated with viral infections.

In at least certain examples, the compounds are substituted diaryloxindole compounds and more particularly, substituted diphenyloxindole compounds. In certain examples, the diaryloxindole compounds of the present invention are effective to inhibit translation. In certain examples, the diaryloxindole compounds of the present invention are effective to deplete intracellular calcium stores. In another example, diaryloxindole compounds are effective to inhibit cellular proliferation. In another example, diaryloxindole compounds are effective to inhibit viral infections. In another example, diaryloxindole compounds are effective to treat or relieve symptoms associated with proliferative disorders, non-proliferative, degenerative disorders and/or viral infections.

The compounds of the present invention are of the type set forth in Formula I.

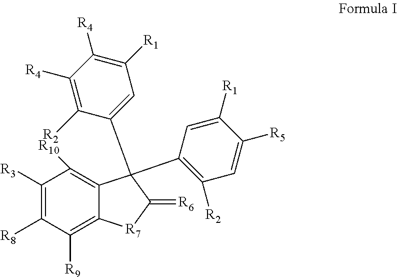

Compounds within the scope of Formula I are those where R₁ is the same or different at its respective location on the compound of Formula I and is selected from hydrogen, halogen, substituted or unsubstituted alkyl or heteroalkyl, substituted or unsubstituted alkenyl or heteroalkenyl, substituted or unsubstituted alkynyl or heteroalkynyl, cycloalkyl, —OR₁₁ where R₁₁ is substituted or unsubstituted alkyl or heteroalkyl, substituted or unsubstituted alkenyl or heteroalkenyl, or substituted or unsubstituted alkynyl or heteroalkynyl; exemplary R₁ is hydrogen, fluorine, chlorine, bromine, iodine, methyl, ethyl, propyl, n-propyl, isopropyl, butyl, n-butyl, isobutyl, tert-butyl, sec-butyl, pentyl, n-pentyl, cyclopentyl, hexyl, n-hexyl, cyclohexyl, heptyl, n-heptyl, cycloheptyl, methoxy, ethoxy, propoxy, butoxy, pentoxy, hexoxy, heptoxy or 2-oxy-3,4,5-trihydroxy-6-(hydroxymethyl)tetahydro-2H-pyran-2-yl;

R₂ is the same or different at its respective location on the compound of Formula I and is selected from hydrogen, hydroxy, alkoxy, substituted alkoxy, methoxy, —O—CH₂—R₁₂, ethoxy, —O—CH₂—CH₂—R₂₁, —O—R₂₂, propargyloxy, 1H-1,2,3-triazol-1-yl, 4-(pyridin-2-ylmethyl)-1H-1,2,3-triazol-1-yl, 4-((5-(2-hydroxyethylamino)pyridin-2-yl)methyl)-1H-1,2,3-triazol-1-yl) wherein R₁₂ is 1H-1,2,3-triazol-4-yl, 1-(2-morpholinoethyl)-1H-1,2,3-triazol-4-yl, carboxymethylene, 2-acetamide, N-(2-hydroxyethyl)-2-acetamido, N-(2-(3,4,5-trihydroxy-6-(hydroxymethyl)tetrahydro-2H-pyran-2-yloxy)ethyl)acetamido, wherein R₂₁ is hydroxy, methoxy, amino, N-methylamino, N,N-dimethylamino, N,N-diethylamino, [N-methyl,N-ethyl]amino, bromo, acetoxy, N-morpholino, N-pyrrolidino, N-piperidino, N-piperazino, N-[4-methyl]-piperazino, ((3R,4S,5S,6R)-2-oxy-3,4,5-trihydroxy-6-(hydroxymethyl)tetrahydro-2H-pyran-2-yl) or 2-oxy-3,4,5-trihydroxy-6-(hydroxymethyl)tetrahydro-2H-pyran-2-yl), and wherein R₂₂ is substituted or unsubstituted alkyl or heteroalkyl, substituted or unsubstituted alkenyl or heteroalkenyl, or substituted or unsubstituted alkynyl or heteroalkynyl, heterocycloalkyl linked to O with an alkyl, alkenyl or alkynyl group, branched or unbranched. Exemplary R₂ is hydrogen, hydroxy, methoxy, substituted methoxy, ethoxy, substituted ethoxy, N-(2-ethoxy)-morpholino, 2-(N,N-dimethylamino)ethoxy, N-(2-ethoxy)-piperazin-1-yl, (1-(N-2-ethyl-morpholino)-1H-1,2,3-triazol-5-yl)methoxy, 4-((5-((2-hydroxyethyl)amino)pyridin-2-yl)methyl)-1H-1,2,3-triazol-1-yl, 4-(pyridin-2-ylmethyl)-1H-1,2,3-triazol-1-yl, prop-2-ynyloxy, N-(3,4,5-trihydroxy-6-(hydroxymethyl)tetrahydro-2H-pyran-2-yl)oxy)ethyl)-2-oxyacetamido, 2-oxyacetic acid, 2-oxyethanol, 2-methoxyethoxy, 2-acetoxyethyl, 2-bromoethoxy, 2-acetoxyethoxy, ethyl 2-oxyacetate, 2-(N,N-dimethylamino)ethoxy, 2-(N,N-diethylamino)ethoxy, 2-(pyrrolidin-1-yl)ethoxy, 2-(piperidin-1-yl)ethoxy, 2-morpholinoethoxy, 2-(piperazin-1-yl)ethoxy, or 2-(4-methylpiperazin-1-yl)ethoxy;

R₃ is hydrogen, halogen, —NH₂, morpholino, morpholinoalkyl, morpholinomethyl, morpholinoethyl, pyridino, 6-morpholinopyridin-3-yl, —OR₁₂ where R₁₂ is substituted or unsubstituted alkyl or heteroalkyl, substituted or unsubstituted alkenyl or heteroalkenyl, or substituted or unsubstituted alkynyl, heteroalkynyl or R₃ is connected to R₁₀ wherein R₃—X—R₁₀ is S—C═N or N═C—S, such as thiazolo. Exemplary R₃ is hydrogen, fluorine, chlorine, bromine, iodine, trifluoroacetyl, 6-morpholinopyridin-3-yl, —NH₂, methoxy, ethoxy, propoxy, butoxy, pentoxy, hexoxy, or heptoxy;

R₄ is the same or different at its respective location on the compound of Formula I and is selected from hydrogen, hydroxy, alkyl, methyl, ethyl, halogen, —N(CH₃)₂, alkoxy, heteroalkyl, or —OR₁₂ where R₁₂ is substituted or unsubstituted alkyl or heteroalkyl, substituted or unsubstituted alkenyl or heteroalkenyl, or substituted or unsubstituted alkynyl or heteroalkynyl. Exemplary R₄ is hydrogen, hydroxyl, methyl, fluorine, chlorine, bromine, iodine, —N(CH₃)₂, methoxy, ethoxy, propoxy, butoxy, pentoxy, hexoxy, heptoxy, morpholinomethyl, 2-oxy-3,4,5-trihydroxy-6-(hydroxymethyl)tetrahydro-2H-pyran-2-yl, or N-(2-(3,4,5-trihydroxy-6-(hydroxymethyl)tetrahydro-2H-pyran-2-yloxy)ethyl)acetamido;

R₅ is hydrogen or hydroxy;

R₆ is oxygen, sulfur, NH or CH₂;

R₇ is oxygen, SH, NH or CH₃;

R₈ is hydrogen, halogen, fluorine, chlorine, bromine, iodine, carboxylate, carboxylate alkyl ester, alkoxycarbonyl or methoxycarbonyl;

R₉ is hydrogen, halogen, fluorine, chlorine, bromine, iodine, carboxylate, carboxylate alkyl ester, alkoxycarbonyl or methoxycarbonyl; R₁₀ is hydrogen or R₁₀ is connected to R₃ wherein R₁₀—X—R₃ is S—C═N or N═C—S.

One of skill will readily understand based on the present disclosure to select a moiety from among the species of each of R₁, R₂, R₃, R₄, R₅, R₆, R₇, R₈, R₉, and R₁₀ to arrive at compounds of the present disclosure. Stated differently, the present specification describes each of the species represented by the Formula I with all of the various combinations of the various moieties for each of R₁, R₂, R₃, R₄, R₅, R₆, R₇, R₈, R₉, and R₁₀ without expressly reciting each species individually. Stated differently, compounds within the scope of Formula I include those being a subset of the combinations of the various moieties for each of R₁, R₂, R₃, R₄, R₅, R₆, R₇, R₈, R₉, and R₁₀. Likewise, one of ordinary skill in the art will readily understand that a particular moiety or moieties can be deselected from among those various moieties for each of R₁, R₂, R₃, R₄, R₅, R₆, R₇, R₈, R₉, and R₁₀. Likewise, one of skill will readily understand that compounds within the scope of the present disclosure can be described using “proviso” language, for example with the proviso that for any particular R group, R is not one or more of the recited species for the R group. For example, compounds include those within the scope of Formula I as described above with the proviso that each of R₁, R₂, R₃, R₄, R₅, R₈, R₉, and R₁₀ are not hydrogen when R₆ is O and R₇ is NH. For example, compounds include those within the scope of Formula I as described above with the proviso that the compound is not compound 1181, 1282, 1289 or 1287.

Alkyl, alkenyl and alkynyl include linear, branched, and cyclic structures and combinations thereof. “Alkyl” includes lower alkyl and extends to cover carbon fragments having up to 20 carbon atoms. Examples of alkyl groups include octyl, nonyl, norbornyl, undecyl, dodecyl, tridecyl, tetradecyl, pentadecyl, eicosyl and the like.

“Lower alkyl” means alkyl groups of from 1 to 7 carbon atoms. Examples of lower alkenyl groups include methyl, ethyl, propyl, isopropyl, butyl, isobutyl, sec-butyl, tert-butyl, pentyl, hexyl, heptyl, cyclopropyl, cyclobutyl, cyclopentyl, cyclohexyl, cycloheptyl and the like.

“Lower alkenyl” means alkenyl groups of 2 to 7 carbon atoms. Examples of lower alkenyl groups include vinyl, allyl, isopropenyl, pentenyl, hexenyl, heptenyl, cyclopropenyl, cyclubutenyl, cyclopentenyl, cyclohexenyl, 1-propenyl, 2-butenyl, 2-methyl-2-butenyl, and the like.

“Lower alkynyl” means alkynyl groups of 2 to 7 carbon atoms. Examples of lower alkynyl groups include ethynyl, propargyl, 3-methyl-1-pentenyl, 2-heptynyl, and the like.

Alternatively, alkenyl and alkynyl groups can be referred to as unsaturated alkyl groups.

“Heteroalkyl” means an alkyl or cycloalkyl including one or more of oxygen, nitrogen or sulfur atoms replace carbon atoms in the alkyl or cycloalkyl group.

“Halogen” means fluorine, chlorine, bromine and iodine.

“Substituted” means one or more hydrogens on an alkyl, alkenyl or alkynyl group are replaced by one or more different atoms or groups of atoms. For example, hydrogen may be substituted by hydroxy.

Some of the compounds described herein contain one or more centers of asymmetry and may give rise to diastereoisomers and optical isomers. The present invention is meant to include such diastereoisomers as well as their racemic and resolved, optically active forms. Optically active (R) and (S) isomers may be resolved using techniques known to those of skill in the art. Some of the compounds described herein contain olefinic double bonds, and unless otherwise specified, are meant to include both E and Z geometric isomers.

In accordance with a method aspect, a method of treating a cellular proliferative disorder by providing and/or administering a diaryloxindole compound described herein to a mammal, e.g., a human or a non-human (e.g., a non-human primate), is provided. In one example, the proliferative disorder is cancer. In accordance with a method aspect, a method of inhibiting translation initiation in cells or inhibiting abnormal proliferation of cells by contacting such cells with a diaryloxindole compound described herein or by providing and/or administering a diaryloxindole compound described herein to a mammal, e.g., a human or a non-human (e.g., a non-human primate), is provided. In accordance with other examples, a method of treating a viral infection by providing and/or administering a diaryloxindole compound of Formula I to a mammal, e.g. a human or a non-human mammal, is provided.

In accordance with an additional aspect, kits are provided for the treatment of (1) cellular proliferative disorders, (2) non-proliferative, degenerative disorders, (3) viral infections, and/or (4) disorders associated with viral infections. In one aspect, the kits comprise a diaryloxindole compound of Formula I, a pharmaceutically acceptable carrier, and optionally, instructions for use. The pharmaceutical composition can be administered to a human subject or a non-human subject depending on the disorder to be treated.

It will be recognized by the person of ordinary skill in the art that the compounds, compositions, methods and kits disclosed herein provide significant advantages over prior technology. Compounds, compositions, methods and kits can be designed or selected to relieve and/or alleviate symptoms in a patient suffering from one or more disorders. These and other aspects and examples are described below.

### BRIEF DESCRIPTION OF THE DRAWINGS

The foregoing and other features and advantages of the present invention will be more fully understood from the following detailed description of illustrative embodiments taken in conjunction with the accompanying drawings.

FIG. 1 depicts a schematic of the anti-cancer mechanism of action for translation initiation inhibitors such as clotrimazole (CLT) and diaryloxindole (DAO).

It will be recognized that the results and examples in the figures are only illustrative and other examples and illustrations will be readily recognized by the person of ordinary skill in the art, given the benefit of this disclosure.

### DETAILED DESCRIPTION OF CERTAIN EMBODIMENTS

In accordance with certain examples, diaryloxindole compounds described herein inhibit translation (e.g., translation initiation). Such compounds are useful for the treatment of (1) cellular proliferative disorders, (2) non-proliferative, degenerative disorders, (3) viral infections, and/or (4) disorders associated with viral infections.

Certain examples are described below with reference to various chemical formulae. The chemical formulae referred to herein can exhibit the phenomena of tautomerism, conformational isomerism, stereo isomerism or geometric isomerism. As the formulae drawings within this specification can represent only one of the possible tautomeric, conformational isomeric, enantiomeric or geometric isomeric forms, it should be understood that the invention encompasses any tautomeric, conformational isomeric, enantiomeric or geometric isomeric forms which exhibit biological or pharmacological activity as described herein.

The compounds and compositions provided below are effective to inhibit translation (e.g., translation initiation) at least to the extent necessary for effective treatment of one or more disorders described herein. The compounds and compositions provided below are effective to inhibit abnormal proliferation of cells. According to embodiments of the present invention, diaryloxindole compounds of the present invention deplete intracellular Ca⁺² which leads to phosphorylation of eIF2α and inhibition of eIF2, a translation initiation factor commonly understood to be necessary for the proliferation of all cancer cells. According to aspects of the present invention, inhibition of translation inhibits cell proliferation, including abnormal proliferation. According to embodiments of the present invention, cell proliferation is common to all forms of cancers and a method treating all forms of cancer is provided by inhibition of cellular proliferation.

While in certain examples translation may be substantially inhibited such that little or no activity results, in other examples the inhibition is at least sufficient to relieve and or alleviate the symptoms from a selected disorder to be treated.

In accordance with certain embodiments, compounds of the invention are represented by the generic formula set forth below.

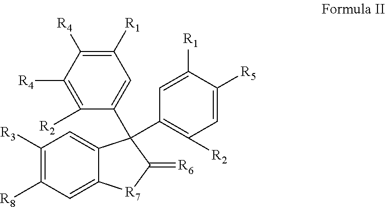

In accordance with certain preferred aspects, R₁ is hydrogen, fluorine, chlorine, bromine, iodine, methyl, ethyl, propyl, n-propyl, isopropyl, butyl, n-butyl, isobutyl, tert-butyl, sec-butyl, pentyl, n-pentyl, cyclopentyl, hexyl, n-hexyl, cyclohexyl, heptyl, n-heptyl, cycloheptyl, methoxy, ethoxy, propoxy, butoxy, pentoxy, hexoxy, heptoxy or 2-oxy-3,4,5-trihydroxy-6-(hydroxymethyl)tetahydro-2H-pyran-2-yl.

R₂ is the same or different and is selected from hydrogen, hydroxy, alkoxy, substituted alkoxy, methoxy, —O—CH₂—R₁₂, ethoxy, —O—CH₂—CH₂—R₂₁, propargyloxy, 1H-1,2,3-triazol-1-yl, 4-(pyridin-2-ylmethyl)-1H-1,2,3-triazol-1-yl, 4-((5-(2-hydroxyethylamino)pyridin-2-yl)methyl)-1H-1,2,3-triazol-1-yl) wherein R₁₂ is 1H-1,2,3-triazol-4-yl, 1-(2-morpholinoethyl)-1H-1,2,3-triazol-4-yl, carboxymethylene, 2-acetamide, N-(2-hydroxyethyl)-2-acetamido, N-(2-(3,4,5-trihydroxy-6-(hydroxymethyl)tetrahydro-2H-pyran-2-yloxy)ethyl)acetamido, wherein R₂₁ is hydroxy, methoxy, amino, N-methylamino, N,N-dimethylamino, N,N-diethylamino, [N-methyl,N-ethyl]amino, bromo, acetoxy, N-morpholino, N-pyrrolidino, N-piperidino, N-piperazino, N-[4-methyl]-piperazino, ((3R,4S,5S,6R)-2-oxy-3,4,5-trihydroxy-6-(hydroxymethyl)tetrahydro-2H-pyran-2-yl) or 2-oxy-3,4,5-trihydroxy-6-(hydroxymethyl)tetrahydro-2H-pyran-2-yl). Exemplary R₂ is hydrogen, hydroxy, methoxy, substituted methoxy, ethoxy, substituted ethoxy, N-(2-ethoxy)-morpholino, 2-(N,N-dimethylamino)ethoxy, N-(2-ethoxy)-piperazin-1-yl, (1-(N-2-ethyl-morpholino)-1H-1,2,3-triazol-5-yl)methoxy, 4-((5-((2-hydroxyethyl)amino)pyridin-2-yl)methyl)-1H-1,2,3-triazol-1-yl, 4-(pyridin-2-ylmethyl)-1H-1,2,3-triazol-1-yl, prop-2-ynyloxy, N-(3,4,5-trihydroxy-6-(hydroxymethyl)tetrahydro-2H-pyran-2-yl)oxy)ethyl)-2-oxyacetamido, 2-oxyacetic acid, 2-oxyethanol, 2-methoxyethoxy, 2-acetoxyethyl, 2-bromoethoxy, 2-acetoxyethoxy, ethyl 2-oxyacetate, 2-(N,N-dimethylamino)ethoxy, 2-(N,N-diethylamino)ethoxy, 2-(pyrrolidin-1-yl)ethoxy, 2-(piperidin-1-yl)ethoxy, 2-morpholinoethoxy, 2-(piperazin-1-yl)ethoxy, or 2-(4-methylpiperazin-1-yl)ethoxy.

R₃ is hydrogen, fluorine, chlorine, bromine, iodine, trifluoroacetyl, 6-morpholinopyridin-3-yl, —NH₂, methoxy, ethoxy, propoxy, butoxy, pentoxy, hexoxy, or heptoxy.

R₄ is the same or different and is selected from hydrogen, hydroxy, methyl, fluorine, chlorine, bromine, iodine, —N(CH₃)₂, methoxy, ethoxy, propoxy, butoxy, pentoxy, hexoxy, heptoxy, morpholinomethyl, 2-oxy-3,4,5-trihydroxy-6-(hydroxymethyl)tetrahydro-2H-pyran-2-yl, or N-(2-(3,4,5-trihydroxy-6-(hydroxymethyl)tetrahydro-2H-pyran-2-yloxy)ethyl)acetamido.

R₅ is hydrogen or hydroxyl; R₆ is oxygen, sulfur, NH, or CH₂; R₇ is O, SH, NH or CH₃; R₈ is hydrogen, halogen, fluorine, chlorine, bromine, iodine, carboxylate, carboxylate alkyl ester, alkoxycarbonyl or methoxycarbonyl.

One of skill will readily understand based on the present disclosure to select a moiety from among the species of each of R₁, R₂, R₃, R₄, R₅, R₆, R₇, and R₈ to arrive at compounds of the present disclosure. One of skill will also readily understand and envision that subsets of the species of one or more of R₁, R₂, R₃, R₄, R₅, R₆, R₇, and R₈ are contemplated, and that one of skill will readily be able to identify subsets of species of one or more of R₁, R₂, R₃, R₄, R₅, R₆, R₇, and R₈ as desired to produce alternate compound formulae of various scope. The present specification describes each of the species represented by the Formula II with all of the various combinations of the various moieties for each of R₁, R₂, R₃, R₄, R₅, R₆, R₇, and R₈ without expressly reciting each species individually. Compounds within the scope of Formula II include those being a subset of the combinations of the various moieties for each of R₁, R₂, R₃, R₄, R₅, R₆, R₇, and R₈. Likewise, one of ordinary skill in the art will readily understand that a particular moiety or moieties can be deselected from among those various moieties for each of R₁, R₂, R₃, R₄, R₅, R₆, R₇, and R₈. Likewise, one of skill will readily understand that compounds within the scope of the present disclosure can be described using “proviso” language, for example with the proviso that for any particular R group, R is not one or more of the recited species for the R group. For example, compounds include those within the scope of Formula II as described above with the proviso that each of R₁, R₂, R₃, R₄, R₅, and R₈ are not hydrogen when R₆ is O and R₇ is NH. For example, compounds include those within the scope of Formula II as described above with the proviso that the compound is not compound 1181, 1282, 1289 or 1287.

Exemplary compounds within the scope of formula II include those where R₁ is halogen, alkyl, —OR₁₂ where R₁₂ is alkyl; R₂ is hydroxy, methoxy, substituted methoxy, ethoxy, substituted ethoxy, N-(2-ethoxy)-morpholino, 2-(N,N-dimethylamino)ethoxy, N-(2-ethoxy)-piperazin-1-yl, (1-(N-2-ethyl-morpholino)-1H-1,2,3-triazol-5-yl)methoxy, 4-((5-((2-hydroxyethyl)amino)pyridin-2-yl)methyl)-1H-1,2,3-triazol-1-yl, 4-(pyridin-2-ylmethyl)-1H-1,2,3-triazol-1-yl, prop-2-ynyloxy, N-(3,4,5-trihydroxy-6-(hydroxymethyl)tetrahydro-2H-pyran-2-yl)oxy)ethyl)-2-oxyacetamido, 2-oxyacetic acid, 2-oxyethanol, 2-methoxyethoxy, 2-acetoxyethyl, 2-bromoethoxy, 2-acetoxyethoxy, ethyl 2-oxyacetate, 2-(N,N-dimethylamino)ethoxy, 2-(N,N-diethylamino)ethoxy, 2-(pyrrolidin-1-yl)ethoxy, 2-(piperidin-1-yl)ethoxy, 2-morpholinoethoxy, 2-(piperazin-1-yl)ethoxy, or 2-(4-methylpiperazin-1-yl)ethoxy; R₃ is hydrogen, R₄ is hydrogen, halogen or alkoxy; R₅ is hydrogen or hydroxyl; R₆ is O; R₇ is NH; and R₈ is hydrogen with the proviso that the compound is not compound 1181, 1282, 1289 or 1287.

Specific compounds within the scope of the present invention include the following compounds.

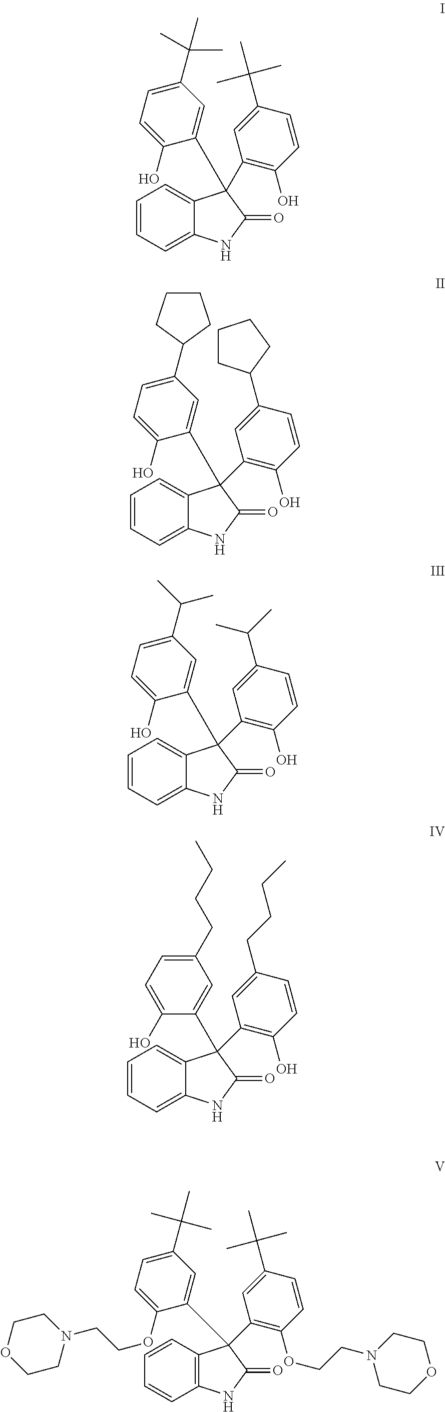

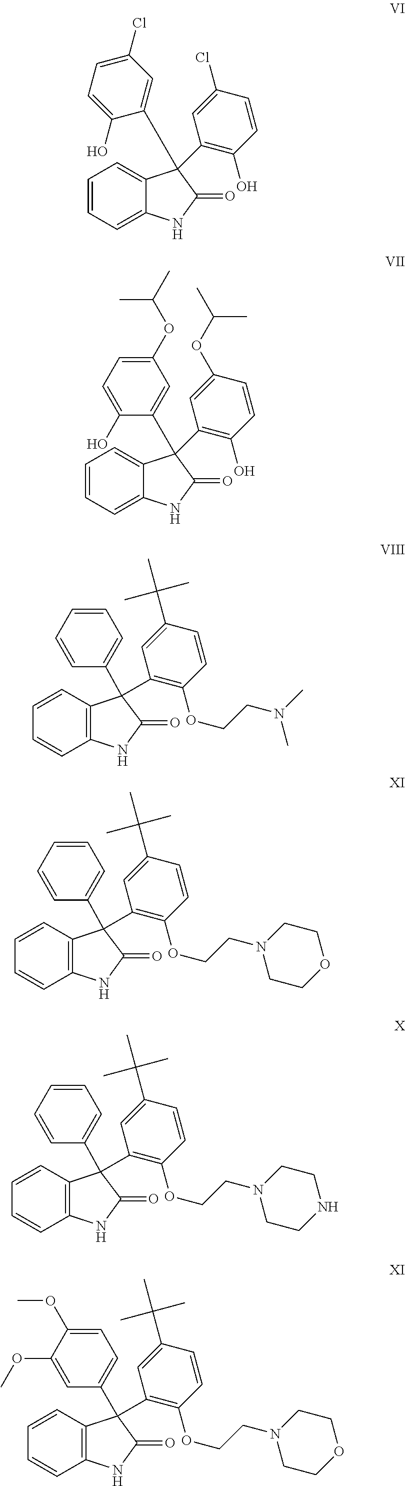

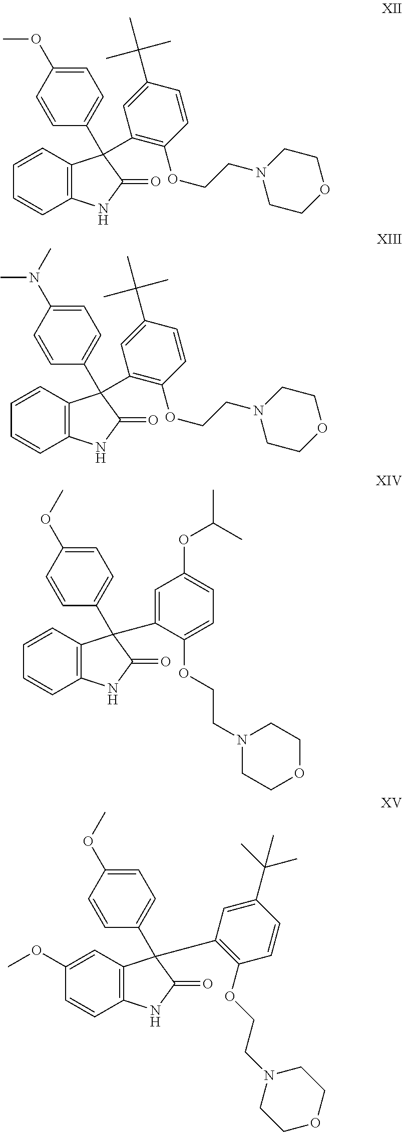

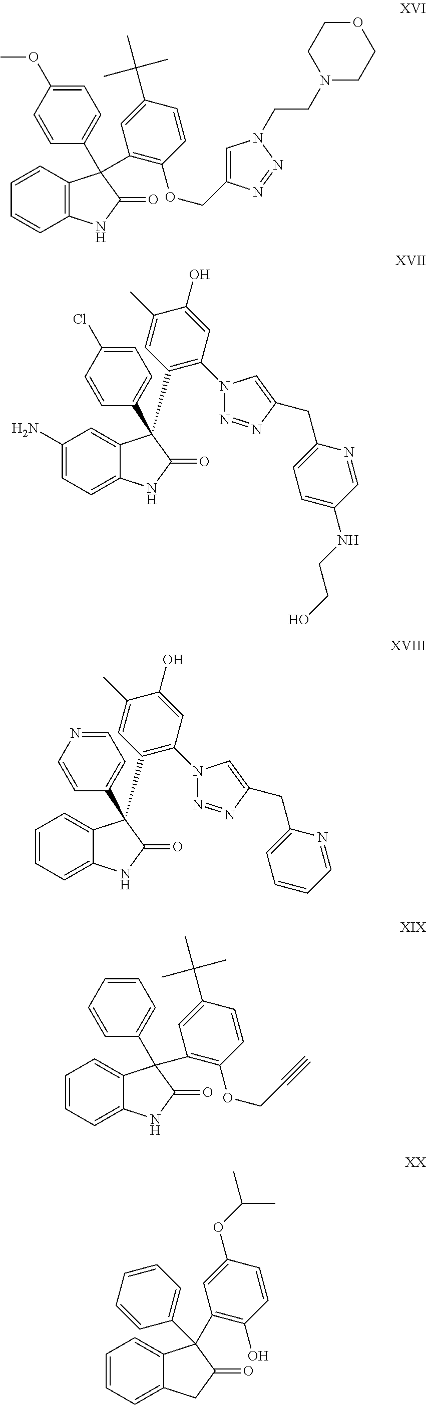 [SMILES: COc1ccc(C2(c3cc(C(C)(C)C)ccc3OCc3cn(CCN4CCOCC4)nn3)C(=O)Nc3ccccc32)cc1 ; Cc1cc([C@]2(c3ccc(Cl)cc3)C(=O)Nc3ccc(N)cc32)c(-n2cc(Cc3ccc(NCCO)cn3)nn2)cc1O ; Cc1cc([C@]2(c3ccncc3)C(=O)Nc3ccccc32)c(-n2cc(Cc3ccccn3)nn2)cc1O ; C#CCOc1ccc(C(C)(C)C)cc1C1(c2ccccc2)C(=O)Nc2ccccc21 ; CC(C)Oc1ccc(O)c(C2(c3ccccc3)C(=O)Cc3ccccc32)c1]

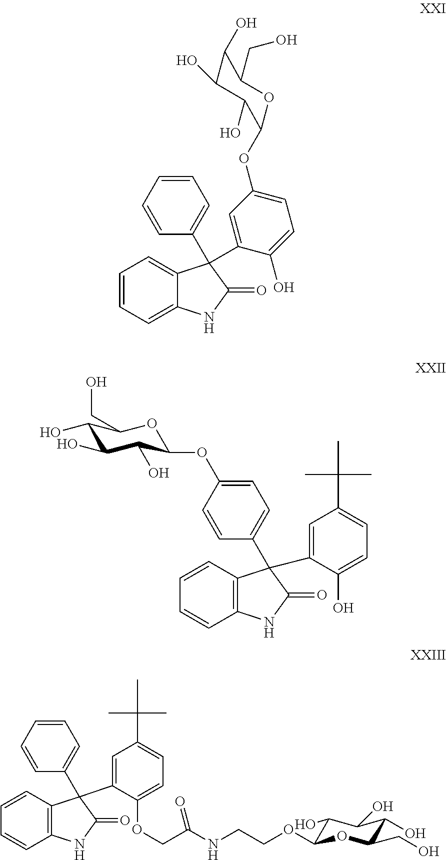

Certain compounds within the scope of the present disclosure include those of Formula III

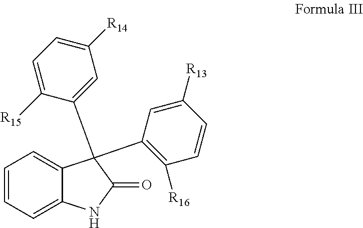

As shown in Formula III, R₁₃ is hydrogen, fluorine, chlorine, bromine, iodine, methyl, ethyl, propyl, n-propyl, isopropyl, butyl, n-butyl, isobutyl, tert-butyl, sec-butyl, pentyl, n-pentyl, cyclopentyl, hexyl, n-hexyl, cyclohexyl, heptyl, n-heptyl, cycloheptyl, methoxy, ethoxy, propoxy, butoxy, pentoxy, hexoxy, heptoxy or 2-oxy-3,4,5-trihydroxy-6-(hydroxymethyl)tetahydro-2H-pyran-2-yl.

R₁₄ is hydrogen, fluorine, chlorine, bromine, iodine, methyl, ethyl, propyl, n-propyl, isopropyl, butyl, n-butyl, isobutyl, tert-butyl, sec-butyl, pentyl, n-pentyl, cyclopentyl, hexyl, n-hexyl, cyclohexyl, heptyl, n-heptyl, cycloheptyl, methoxy, ethoxy. propoxy, butoxy, pentoxy, hexoxy, heptoxy or 2-oxy-3,4,5-trihydroxy-6-(hydroxymethyl)tetahydro-2H-pyran-2-yl.

R₁₅ is hydrogen, hydroxyl, alkoxy, substituted alkoxy, methoxy, —O—CH₂—R₁₂, ethoxy, —O—CH₂—CH₂—R₂₁, propargyloxy, 1H-1,2,3-triazol-1-yl, 4-(pyridin-2-ylmethyl)-1H-1,2,3-triazol-1-yl, 4-((5-(2-hydroxyethylamino)pyridin-2-yl)methyl)-1H-1,2,3-triazol-1-yl) wherein R₁₂ is 1H-1,2,3-triazol-4-yl, 1-(2-morpholinoethyl)-1H-1,2,3-triazol-4-yl, carboxymethylene, 2-acetamide, N-(2-hydroxyethyl)-2-acetamido, N-(2-(3,4,5-trihydroxy-6-(hydroxymethyl)tetrahydro-2H-pyran-2-yloxy)ethyl)acetamido, wherein R₂₁ is hydroxy, methoxy, amino, N-methylamino, N,N-dimethylamino, N,N-diethylamino, [N-methyl,N-ethyl]amino, bromo, acetoxy, N-morpholino, N-pyrrolidino, N-piperidino, N-piperazino, N-[4-methyl]-piperazino, ((3R,4S,5S,6R)-2-oxy-3,4,5-trihydroxy-6-(hydroxymethyl)tetrahydro-2H-pyran-2-yl) or 2-oxy-3,4,5-trihydroxy-6-(hydroxymethyl)tetrahydro-2H-pyran-2-yl). Exemplary R₁₅ is hydrogen, hydroxy, methoxy, substituted methoxy, ethoxy, substituted ethoxy, N-(2-ethoxy)-morpholino, 2-(N,N-dimethylamino)ethoxy, N-(2-ethoxy)-piperazin-1-yl, (1-(N-2-ethyl-morpholino)-1H-1,2,3-triazol-5-yl)methoxy, 4-((5-((2-hydroxyethyl)amino)pyridin-2-yl)methyl)-1H-1,2,3-triazol-1-yl, 4-(pyridin-2-ylmethyl)-1H-1,2,3-triazol-1-yl, prop-2-ynyloxy, N-(3,4,5-trihydroxy-6-(hydroxymethyl)tetrahydro-2H-pyran-2-yl)oxy)ethyl)-2-oxyacetamido, 2-oxyacetic acid, 2-oxyethanol, 2-methoxyethoxy, 2-acetoxyethyl, 2-bromoethoxy, 2-acetoxyethoxy, ethyl 2-oxyacetate, 2-(N,N-dimethylamino)ethoxy, 2-(N,N-diethylamino)ethoxy, 2-(pyrrolidin-1-yl)ethoxy, 2-(piperidin-1-yl)ethoxy, 2-morpholinoethoxy, 2-(piperazin-1-yl)ethoxy, or 2-(4-methylpiperazin-1-yl)ethoxy.

R₁₆ is hydrogen, hydroxy, alkoxy, substituted alkoxy, methoxy, —O—CH₂—R₁₂, ethoxy, —O—CH₂—CH₂—R₂₁, propargyloxy, 1H-1,2,3-triazol-1-yl, 4-(pyridin-2-ylmethyl)-1H-1,2,3-triazol-1-yl, 4-((5-(2-hydroxyethylamino)pyridin-2-yl)methyl)-1H-1,2,3-triazol-1-yl) wherein R₁₂ is 1H-1,2,3-triazol-4-yl, 1-(2-morpholinoethyl)-1H-1,2,3-triazol-4-yl, carboxymethylene, 2-acetamide, N-(2-hydroxyethyl)-2-acetamido, N-(2-(3,4,5-trihydroxy-6-(hydroxymethyl)tetrahydro-2H-pyran-2-yloxy)ethyl)acetamido, wherein R₂₁ is hydroxy, methoxy, amino, N-methylamino, N,N-dimethylamino, N,N-diethylamino, [N-methyl,N-ethyl]amino, bromo, acetoxy, N-morpholino, N-pyrrolidino, N-piperidino, N-piperazino, N-[4-methyl]-piperazino, ((3R,4S,5S,6R)-2-oxy-3,4,5-trihydroxy-6-(hydroxymethyl)tetrahydro-2H-pyran-2-yl) or 2-oxy-3,4,5-trihydroxy-6-(hydroxymethyl)tetrahydro-2H-pyran-2-yl). Exemplary R₁₆ is hydrogen, hydroxy, methoxy, substituted methoxy, ethoxy, substituted ethoxy, N-(2-ethoxy)-morpholino, 2-(N,N-dimethylamino)ethoxy, N-(2-ethoxy)-piperazin-1-yl, (1-(N-2-ethyl-morpholino)-1H-1,2,3-triazol-5-yl)methoxy, 4-((5-((2-hydroxyethyl)amino)pyridin-2-yl)methyl)-1H-1,2,3-triazol-1-yl, 4-(pyridin-2-ylmethyl)-1H-1,2,3-triazol-1-yl, prop-2-ynyloxy, N-(3,4,5-trihydroxy-6-(hydroxymethyl)tetrahydro-2H-pyran-2-yl)oxy)ethyl)-2-oxyacetamido, 2-oxyacetic acid, 2-oxyethanol, 2-methoxyethoxy, 2-acetoxyethyl, 2-bromoethoxy, 2-acetoxyethoxy, ethyl 2-oxyacetate, 2-(N,N-dimethylamino)ethoxy, 2-(N,N-diethylamino)ethoxy, 2-(pyrrolidin-1-yl)ethoxy, 2-(piperidin-1-yl)ethoxy, 2-morpholinoethoxy, 2-(piperazin-1-yl)ethoxy, or 2-(4-methylpiperazin-1-yl)ethoxy.

One of skill will readily understand based on the present disclosure to select a moiety from among the species of each of R₁₃, R₁₄, R₁₅, and R₁₆ to arrive at compounds of the present disclosure. One of skill will also readily understand and envision that subsets of the species of one or more of R₁₃, R₁₄, R₁₅, and R₁₆ are contemplated, and that one of skill will readily be able to identify subsets of species of one or more of R₁₃, R₁₄, R₁₅, and R₁₆ as desired to produce alternate compound formulae of various scope The present specification describes each of the species represented by the Formula III with all of the various combinations of the various moieties for each of R₁₃, R₁₄, R₁₅, and R₁₆ without expressly reciting each species individually. Compounds within the scope of Formula III include those being a subset of the combinations of the various moieties for each of R₁₃, R₁₄, R₁₅, and R₁₆. Likewise, one of ordinary skill in the art will readily understand that a particular moiety or moieties can be deselected from among those various moieties for each of R₁₃, R₁₄, R₁₅, and R₁₆. Likewise, one of skill will readily understand that compounds within the scope of the present disclosure can be described using “proviso” language, for example with the proviso that for any particular R group, R is not one or more of the recited species for the R group. For example, compounds include those within the scope of Formula III as described above with the proviso that each of R₁₃, R₁₄, R₁₅, and R₁₆ are not hydrogen. For example, compounds include those within the scope of Formula III as described above with the proviso that the compound is not compound 1181, 1282, 1289 or 1287.

Exemplary compounds include those where R₁₃ is fluorine, chlorine, bromine, iodine, methyl, ethyl, propyl, n-propyl, isopropyl, butyl, n-butyl, isobutyl, tert-butyl, sec-butyl, pentyl, n-pentyl, cyclopentyl, hexyl, n-hexyl, cyclohexyl, heptyl, n-heptyl, cycloheptyl, methoxy, ethoxy, propoxy, butoxy, pentoxy, hexoxy, or heptoxy; R₁₄ is fluorine, chlorine, bromine, iodine, methyl, ethyl, propyl, n-propyl, isopropyl, butyl, n-butyl, isobutyl, tert-butyl, sec-butyl, pentyl, n-pentyl, cyclopentyl, hexyl, n-hexyl, cyclohexyl, heptyl, n-heptyl, cycloheptyl, methoxy, ethoxy, propoxy, butoxy, pentoxy, hexoxy, or heptoxy; R₁₅ is hydrogen, hydroxy, methoxy, substituted methoxy, ethoxy, substituted ethoxy, N-(2-ethoxy)-morpholino, 2-(N,N-dimethylamino)ethoxy, N-(2-ethoxy)-piperazin-1-yl, (1-(N-2-ethyl-morpholino)-1H-1,2,3-triazol-5-yl)methoxy, 4-((5-((2-hydroxyethyl)amino)pyridin-2-yl)methyl)-1H-1,2,3-triazol-1-yl, 4-(pyridin-2-ylmethyl)-1H-1,2,3-triazol-1-yl, prop-2-ynyloxy, N-(3,4,5-trihydroxy-6-(hydroxymethyl)tetrahydro-2H-pyran-2-yl)oxy)ethyl)-2-oxyacetamido, 2-oxyacetic acid, 2-oxyethanol, 2-methoxyethoxy, 2-acetoxyethyl, 2-bromoethoxy, 2-acetoxyethoxy, ethyl 2-oxyacetate, 2-(N,N-dimethylamino)ethoxy, 2-(N,N-diethylamino)ethoxy, 2-(pyrrolidin-1-yl)ethoxy, 2-(piperidin-1-yl)ethoxy, 2-morpholinoethoxy, 2-(piperazin-1-yl)ethoxy, or 2-(4-methylpiperazin-1-yl)ethoxy; R₁₆ is hydrogen, hydroxy, methoxy, substituted methoxy, ethoxy, substituted ethoxy, N-(2-ethoxy)-morpholino, 2-(N,N-dimethylamino)ethoxy, N-(2-ethoxy)-piperazin-1-yl, (1-(N-2-ethyl-morpholino)-1H-1,2,3-triazol-5-yl)methoxy, 4-((5-((2-hydroxyethyl)amino)pyridin-2-yl)methyl)-1H-1,2,3-triazol-1-yl, 4-(pyridin-2-ylmethyl)-1H-1,2,3-triazol-1-yl, prop-2-ynyloxy, N-(3,4,5-trihydroxy-6-(hydroxymethyl)tetrahydro-2H-pyran-2-yl)oxy)ethyl)-2-oxyacetamido, 2-oxyacetic acid, 2-oxyethanol, 2-methoxyethoxy, 2-acetoxyethyl, 2-bromoethoxy, 2-acetoxyethoxy, ethyl 2-oxyacetate, 2-(N,N-dimethylamino)ethoxy, 2-(N,N-diethylamino)ethoxy, 2-(pyrrolidin-1-yl)ethoxy, 2-(piperidin-1-yl)ethoxy, 2-morpholinoethoxy, 2-(piperazin-1-yl)ethoxy, or 2-(4-methylpiperazin-1-yl)ethoxy.

Exemplary compounds include those where R₁₃ is fluorine, chlorine, bromine, iodine, methyl, ethyl, propyl, n-propyl, isopropyl, butyl, n-butyl, isobutyl, tert-butyl, sec-butyl, pentyl, n-pentyl, cyclopentyl, hexyl, n-hexyl, cyclohexyl, heptyl, n-heptyl, cycloheptyl, methoxy, ethoxy, propoxy, butoxy, pentoxy, hexoxy, or heptoxy; R₁₄ is hydrogen; R₁₅ is hydrogen; R₁₆ is hydrogen, hydroxy, methoxy, substituted methoxy, ethoxy, substituted ethoxy, N-(2-ethoxy)-morpholino, 2-(N,N-dimethylamino)ethoxy, N-(2-ethoxy)-piperazin-1-yl, (1-(N-2-ethyl-morpholino)-1H-1,2,3-triazol-5-yl)methoxy, 4-((5-((2-hydroxyethyl)amino)pyridin-2-yl)methyl)-1H-1,2,3-triazol-1-yl, 4-(pyridin-2-ylmethyl)-1H-1,2,3-triazol-1-yl, prop-2-ynyloxy, N-(3,4,5-trihydroxy-6-(hydroxymethyl)tetrahydro-2H-pyran-2-yl)oxy)ethyl)-2-oxyacetamido, 2-oxyacetic acid, 2-oxyethanol, 2-methoxyethoxy, 2-acetoxyethyl, 2-bromoethoxy, 2-acetoxyethoxy, ethyl 2-oxyacetate, 2-(N,N-dimethylamino)ethoxy, 2-(N,N-diethylamino)ethoxy, 2-(pyrrolidin-1-yl)ethoxy, 2-(piperidin-1-yl)ethoxy, 2-morpholinoethoxy, 2-(piperazin-1-yl)ethoxy, or 2-(4-methylpiperazin-1-yl)ethoxy.

Certain compounds within the scope of the present disclosure include those of Formula IV.

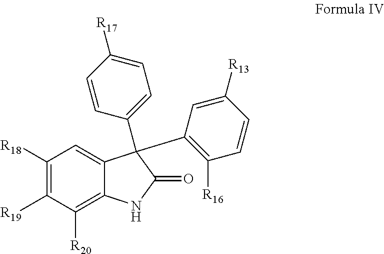

As shown in Formula IV, R₁₃ is hydrogen, fluorine, chlorine, bromine, iodine, methyl, ethyl, propyl, n-propyl, isopropyl, butyl, n-butyl, isobutyl, tert-butyl, sec-butyl, pentyl, n-pentyl, cyclopentyl, hexyl, n-hexyl, cyclohexyl, heptyl, n-heptyl, cycloheptyl, methoxy, ethoxy, propoxy, butoxy, pentoxy, hexoxy, heptoxy or 2-oxy-3,4,5-trihydroxy-6-(hydroxymethyl)tetahydro-2H-pyran-2-yl.

R₁₆ is hydrogen, hydroxy, alkoxy, substituted alkoxy, methoxy, —O—CH₂—R₁₂, ethoxy, —O—CH₂—CH₂—R₂₁, propargyloxy, 1H-1,2,3-triazol-1-yl, 4-(pyridin-2-ylmethyl)-1H-1,2,3-triazol-1-yl, 4-((5-(2-hydroxyethylamino)pyridin-2-yl)methyl)-1H-1,2,3-triazol-1-yl) wherein R₁₂ is 1H-1,2,3-triazol-4-yl, 1-(2-morpholinoethyl)-1H-1,2,3-triazol-4-yl, carboxymethylene, 2-acetamide, N-(2-hydroxyethyl)-2-acetamido, N-(2-(3,4,5-trihydroxy-6-(hydroxymethyl)tetrahydro-2H-pyran-2-yloxy)ethyl)acetamido, wherein R₂₁ is hydroxy, methoxy, amino, N-methylamino, N,N-dimethylamino, N,N-diethylamino, [N-methyl,N-ethyl]amino, bromo, acetoxy, N-morpholino, N-pyrrolidino, N-piperidino, N-piperazino, N-[4-methyl]-piperazino, ((3R,4S,5S,6R)-2-oxy-3,4,5-trihydroxy-6-(hydroxymethyl)tetrahydro-2H-pyran-2-yl) or 2-oxy-3,4,5-trihydroxy-6-(hydroxymethyl)tetrahydro-2H-pyran-2-yl). Exemplary R₁₆ is hydrogen, hydroxy, methoxy, substituted methoxy, ethoxy, substituted ethoxy, N-(2-ethoxy)-morpholino, 2-(N,N-dimethylamino)ethoxy, N-(2-ethoxy)-piperazin-1-yl, (1-(N-2-ethyl-morpholino)-1H-1,2,3-triazol-5-yl)methoxy, 4-((5-((2-hydroxyethyl)amino)pyridin-2-yl)methyl)-1H-1,2,3-triazol-1-yl, 4-(pyridin-2-ylmethyl)-1H-1,2,3-triazol-1-yl, prop-2-ynyloxy, N-(3,4,5-trihydroxy-6-(hydroxymethyl)tetrahydro-2H-pyran-2-yl)oxy)ethyl)-2-oxyacetamido, 2-oxyacetic acid, 2-oxyethanol, 2-methoxyethoxy, 2-acetoxyethyl, 2-bromoethoxy, 2-acetoxyethoxy, ethyl 2-oxyacetate, 2-(N,N-dimethylamino)ethoxy, 2-(N,N-diethylamino)ethoxy, 2-(pyrrolidin-1-yl)ethoxy, 2-(piperidin-1-yl)ethoxy, 2-morpholinoethoxy, 2-(piperazin-1-yl)ethoxy, or 2-(4-methylpiperazin-1-yl)ethoxy.

R₁₇ is hydrogen, hydroxy, methyl, fluorine, chlorine, bromine, iodine, —N(CH₃)₂, methoxy, ethoxy, propoxy, butoxy, pentoxy, hexoxy, heptoxy, morpholinomethyl, 2-oxy-3,4,5-trihydroxy-6-(hydroxymethyl)tetrahydro-2H-pyran-2-yl, or N-(2-(3,4,5-trihydroxy-6-(hydroxymethyl)tetrahydro-2H-pyran-2-yloxy)ethyl)acetamido.

R₁₈ is hydrogen, fluorine, chlorine, bromine, iodine, trifluoroacetyl, 6-morpholinopyridin-3-yl, —NH₂, methoxy, ethoxy, propoxy, butoxy, pentoxy, hexoxy, or heptoxy.

R₁₉ is hydrogen, halogen, fluorine, chlorine, bromine, iodine, carboxylate, carboxylate alkyl ester, alkoxycarbonyl or methoxycarbonyl.

R₂₀ is hydrogen, halogen, fluorine, chlorine, bromine, iodine, carboxylate, carboxylate alkyl ester, alkoxycarbonyl or methoxycarbonyl.

One of skill will readily understand based on the present disclosure to select a moiety from among the species of each of R₁₃, R₁₆, R₁₇, R₁₈, R₁₉, and R₂₀ to arrive at compounds of the present disclosure. One of skill will also readily understand and envision that subsets of the species of one or more of R₁₃, R₁₆, R₁₇, R₁₈, R₁₉, and R₂₀ are contemplated, and that one of skill will readily be able to identify subsets of species of one or more of R₁₃, R₁₆, R₁₇, R₁₈, R₁₉, and R₂₀ as desired to produce alternate compound formulae of various scope. The present specification describes each of the species represented by the Formula IV with all of the various combinations of the various moieties for each of R₁₃, R₁₆, R₁₇, R₁₈, R₁₉, and R₂₀ without expressly reciting each species individually. Compounds within the scope of Formula IV include those being a subset of the combinations of the various moieties for each of R₁₃, R₁₆, R₁₇, R₁₈, R₁₉, and R₂₀. Likewise, one of ordinary skill in the art will readily understand that a particular moiety or moieties can be deselected from among those various moieties for each of R₁₃, R₁₆, R₁₇, R₁₈, R₁₉, and R₂₀. Likewise, one of skill will readily understand that compounds within the scope of the present disclosure can be described using “proviso” language, for example with the proviso that for any particular R group, R is not one or more of the recited species for the R group. For example, compounds include those within the scope of Formula IV as described above with the proviso that each of R₁₃, R₁₆, R₁₇, R₁₈, R₁₉, and R₂₀ are not hydrogen. For example, compounds include those within the scope of Formula IV as described above with the proviso that the compound is not compound 1181, 1282, 1289 or 1287.

Exemplary compounds include those where R₁₃ is fluorine, chlorine, bromine, iodine, methyl, ethyl, propyl, n-propyl, isopropyl, butyl, n-butyl, isobutyl, tert-butyl, sec-butyl, pentyl, n-pentyl, cyclopentyl, hexyl, n-hexyl, cyclohexyl, heptyl, n-heptyl, cycloheptyl, methoxy, ethoxy, propoxy, butoxy, pentoxy, hexoxy, or heptoxy; R₁₆ hydrogen, hydroxy, methoxy, substituted methoxy, ethoxy, substituted ethoxy, N-(2-ethoxy)-morpholino, 2-(N,N-dimethylamino)ethoxy, N-(2-ethoxy)-piperazin-1-yl, (1-(N-2-ethyl-morpholino)-1H-1,2,3-triazol-5-yl)methoxy, 4-((5-((2-hydroxyethyl)amino)pyridin-2-yl)methyl)-1H-1,2,3-triazol-1-yl, 4-(pyridin-2-ylmethyl)-1H-1,2,3-triazol-1-yl, prop-2-ynyloxy, N-(3,4,5-trihydroxy-6-(hydroxymethyl)tetrahydro-2H-pyran-2-yl)oxy)ethyl)-2-oxyacetamido, 2-oxyacetic acid, 2-oxyethanol, 2-methoxyethoxy, 2-acetoxyethyl, 2-bromoethoxy, 2-acetoxyethoxy, ethyl 2-oxyacetate, 2-(N,N-dimethylamino)ethoxy, 2-(N,N-diethylamino)ethoxy, 2-(pyrrolidin-1-yl)ethoxy, 2-(piperidin-1-yl)ethoxy, 2-morpholinoethoxy, 2-(piperazin-1-yl)ethoxy, or 2-(4-methylpiperazin-1-yl)ethoxy; R₁₇ is methyl, fluorine, chlorine, bromine, iodine, —N(CH₃)₂, methoxy, ethoxy, propoxy, butoxy, pentoxy, hexoxy, heptoxy, morpholinomethyl, 2-oxy-3,4,5-trihydroxy-6-(hydroxymethyl)tetrahydro-2H-pyran-2-yl, or N-(2-(3,4,5-trihydroxy-6-(hydroxymethyl)tetrahydro-2H-pyran-2-yloxy)ethyl)acetamido; R₁₈ is hydrogen, fluorine, chlorine, bromine, iodine, trifluoroacetyl, 6-morpholinopyridin-3-yl, —NH₂, methoxy, ethoxy, propoxy, butoxy, pentoxy, hexoxy, or heptoxy; R₁₉ is hydrogen, fluorine, chlorine, bromine, iodine, or methoxycarbonyl; R₂₀ is hydrogen, fluorine, chlorine, bromine, iodine, carboxylate, or methoxycarbonyl.

Certain compounds within the scope of the present disclosure include those of Formula V.

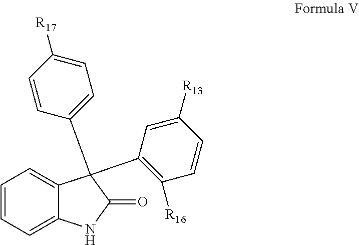

As shown in Formula V, R₁₃ is hydrogen, fluorine, chlorine, bromine, iodine, methyl, ethyl, propyl, n-propyl, isopropyl, butyl, n-butyl, isobutyl, tert-butyl, sec-butyl, pentyl, n-pentyl, cyclopentyl, hexyl, n-hexyl, cyclohexyl, heptyl, n-heptyl, cycloheptyl, methoxy, ethoxy, propoxy, butoxy, pentoxy, hexoxy, heptoxy or 2-oxy-3,4,5-trihydroxy-6-(hydroxymethyl)tetahydro-2H-pyran-2-yl.

R₁₆ is hydrogen, hydroxy, alkoxy, substituted alkoxy, methoxy, —O—CH₂—R₁₂, ethoxy, —O—CH₂—CH₂—R₂₁, propargyloxy, 1H-1,2,3-triazol-1-yl, 4-(pyridin-2-ylmethyl)-1H-1,2,3-triazol-1-yl, 4-((5-(2-hydroxyethylamino)pyridin-2-yl)methyl)-1H-1,2,3-triazol-1-yl) wherein R₁₂ is 1H-1,2,3-triazol-4-yl, 1-(2-morpholinoethyl)-1H-1,2,3-triazol-4-yl, carboxymethylene, 2-acetamide, N-(2-hydroxyethyl)-2-acetamido, N-(2-(3,4,5-trihydroxy-6-(hydroxymethyl)tetrahydro-2H-pyran-2-yloxy)ethyl)acetamido, wherein R₂₁ is hydroxy, methoxy, amino, N-methylamino, N,N-dimethylamino, N,N-diethylamino, [N-methyl,N-ethyl]amino, bromo, acetoxy, N-morpholino, N-pyrrolidino, N-piperidino, N-piperazino, N-[4-methyl]-piperazino, ((3R,4S,5S,6R)-2-oxy-3,4,5-trihydroxy-6-(hydroxymethyl)tetrahydro-2H-pyran-2-yl) or 2-oxy-3,4,5-trihydroxy-6-(hydroxymethyl)tetrahydro-2H-pyran-2-yl). Exemplary R₁₆ is hydrogen, hydroxy, methoxy, substituted methoxy, ethoxy, substituted ethoxy, N-(2-ethoxy)-morpholino, 2-(N,N-dimethylamino)ethoxy, N-(2-ethoxy)-piperazin-1-yl, (1-(N-2-ethyl-morpholino)-1H-1,2,3-triazol-5-yl)methoxy, 4-((5-((2-hydroxyethyl)amino)pyridin-2-yl)methyl)-1H-1,2,3-triazol-1-yl, 4-(pyridin-2-ylmethyl)-1H-1,2,3-triazol-1-yl, prop-2-ynyloxy, N-(3,4,5-trihydroxy-6-(hydroxymethyl)tetrahydro-2H-pyran-2-yl)oxy)ethyl)-2-oxyacetamido, 2-oxyacetic acid, 2-oxyethanol, 2-methoxyethoxy, 2-acetoxyethyl, 2-bromoethoxy, 2-acetoxyethoxy, ethyl 2-oxyacetate, 2-(N,N-dimethylamino)ethoxy, 2-(N,N-diethylamino)ethoxy, 2-(pyrrolidin-1-yl)ethoxy, 2-(piperidin-1-yl)ethoxy, 2-morpholinoethoxy, 2-(piperazin-1-yl)ethoxy, or 2-(4-methylpiperazin-1-yl)ethoxy.

R₁₇ is hydrogen, hydroxy, methyl, fluorine, chlorine, bromine, iodine, —N(CH₃)₂, methoxy, ethoxy, propoxy, butoxy, pentoxy, hexoxy, heptoxy, morpholinomethyl, 2-oxy-3,4,5-trihydroxy-6-(hydroxymethyl)tetrahydro-2H-pyran-2-yl, or N-(2-(3,4,5-trihydroxy-6-(hydroxymethyl)tetrahydro-2H-pyran-2-yloxy)ethyl)acetamido.

One of skill will readily understand based on the present disclosure to select a moiety from among the species of each of R₁₃, R₁₆, and R₁₇ to arrive at compounds of the present disclosure. One of skill will also readily understand and envision that subsets of the species of one or more of R₁₃, R₁₆, and R₁₇ are contemplated, and that one of skill will readily be able to identify subsets of species of one or more of R₁₃, R₁₆, and R₁₇ as desired to produce alternate compound formulae of various scope. The present specification describes each of the species represented by the Formula V with all of the various combinations of the various moieties for each of R₁₃, R₁₆, and R₁₇ without expressly reciting each species individually. Compounds within the scope of Formula V include those being a subset of the combinations of the various moieties for each of R₁₃, R₁₆, and R₁₇. Likewise, one of ordinary skill in the art will readily understand that a particular moiety or moieties can be deselected from among those various moieties for each of R₁₃, R₁₆, and R₁₇. Likewise, one of skill will readily understand that compounds within the scope of the present disclosure can be described using “proviso” language, for example with the proviso that for any particular R group, R is not one or more of the recited species for the R group. For example, compounds include those within the scope of Formula V as described above with the proviso that each of R₁₃, R₁₆, and R₁₇ are not hydrogen. For example, compounds include those within the scope of Formula V as described above with the proviso that the compound is not compound 1181, 1282, 1289 or 1287.

Certain compounds within the scope of the present disclosure include those of Formula VI.

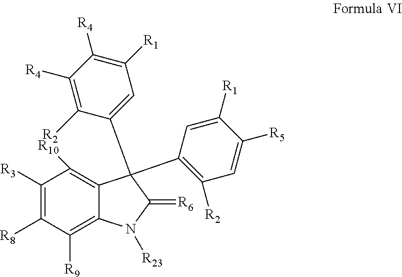

R₁ is the same or different and is selected from hydrogen, halogen, substituted or unsubstituted alkyl or heteroalkyl, substituted or unsubstituted alkenyl or heteroalkenyl, substituted or unsubstituted alkynyl or heteroalkynyl, cycloalkyl, —OR₁₁ where R₁₁ is substituted or unsubstituted alkyl or heteroalkyl, substituted or unsubstituted alkenyl or heteroalkenyl, or substituted or unsubstituted alkynyl or heteroalkynyl; exemplary R₁ is hydrogen, fluorine, chlorine, bromine, iodine, methyl, ethyl, propyl, n-propyl, isopropyl, butyl, n-butyl, isobutyl, tert-butyl, sec-butyl, pentyl, n-pentyl, cyclopentyl, hexyl, n-hexyl, cyclohexyl, heptyl, n-heptyl, cycloheptyl, methoxy, ethoxy, propoxy, butoxy, pentoxy, hexoxy, heptoxy or 2-oxy-3,4,5-trihydroxy-6-(hydroxymethyl)tetahydro-2H-pyran-2-yl;
R₂ is the same or different and is selected from hydrogen, hydroxy, alkoxy, substituted alkoxy, methoxy, —O—CH₂—R₁₂, ethoxy, —O—CH₂—CH₂—R₂₁, —O—R₂₂, propargyloxy, 1H-1,2,3-triazol-1-yl, 4-(pyridin-2-ylmethyl)-1H-1,2,3-triazol-1-yl, 4-((5-(2-hydroxyethylamino)pyridin-2-yl)methyl)-1H-1,2,3-triazol-1-yl) wherein R₁₂ is 1H-1,2,3-triazol-4-yl, 1-(2-morpholinoethyl)-1H-1,2,3-triazol-4-yl, carboxymethylene, 2-acetamide, N-(2-hydroxyethyl)-2-acetamido, N-(2-(3,4,5-trihydroxy-6-(hydroxymethyl)tetrahydro-2H-pyran-2-yloxy)ethyl)acetamido, wherein R₂₁ is hydroxy, methoxy, amino, N-methylamino, N,N-dimethylamino, N,N-diethylamino, [N-methyl,N-ethyl]amino, bromo, acetoxy, N-morpholino, N-pyrrolidino, N-piperidino, N-piperazino, N-[4-methyl]-piperazino, ((3R,4S,5S,6R)-2-oxy-3,4,5-trihydroxy-6-(hydroxymethyl)tetrahydro-2H-pyran-2-yl) or 2-oxy-3,4,5-trihydroxy-6-(hydroxymethyl)tetrahydro-2H-pyran-2-yl), and wherein R₂₂ is substituted or unsubstituted alkyl or heteroalkyl, substituted or unsubstituted alkenyl or heteroalkenyl, or substituted or unsubstituted alkynyl or heteroalkynyl, heterocycloalkyl linked to O with an alkyl, alkenyl or alkynyl group, branched or unbranched. Exemplary R₂ is hydrogen, hydroxy, methoxy, substituted methoxy, ethoxy, substituted ethoxy, N-(2-ethoxy)-morpholino, 2-(N,N-dimethylamino)ethoxy, N-(2-ethoxy)-piperazin-1-yl, (1-(N-2-ethyl-morpholino)-1H-1,2,3-triazol-5-yl)methoxy, 4-((5-((2-hydroxyethyl)amino)pyridin-2-yl)methyl)-1H-1,2,3-triazol-1-yl, 4-(pyridin-2-ylmethyl)-1H-1,2,3-triazol-1-yl, prop-2-ynyloxy, N-(3,4,5-trihydroxy-6-(hydroxymethyl)tetrahydro-2H-pyran-2-yl)oxy)ethyl)-2-oxyacetamido, 2-oxyacetic acid, 2-oxyethanol, 2-methoxyethoxy, 2-acetoxyethyl, 2-bromoethoxy, 2-acetoxyethoxy, ethyl 2-oxyacetate, 2-(N,N-dimethylamino)ethoxy, 2-(N,N-diethylamino)ethoxy, 2-(pyrrolidin-1-yl)ethoxy, 2-(piperidin-1-yl)ethoxy, 2-morpholinoethoxy, 2-(piperazin-1-yl)ethoxy, or 2-(4-methylpiperazin-1-yl)ethoxy;
R₃ is hydrogen, halogen, —NH₂, morpholino, morpholinoalkyl, morpholinomethyl, morpholinoethyl, pyridino, 6-morpholinopyridin-3-yl, —OR₁₂ where R₁₂ is substituted or unsubstituted alkyl or heteroalkyl, substituted or unsubstituted alkenyl or heteroalkenyl, or substituted or unsubstituted alkynyl, heteroalkynyl or R₃ is connected to R₁₀ wherein R₃—X—R₁₀ is S—C═N or N═C—S, such as thiazolo. Exemplary R₃ is hydrogen, fluorine, chlorine, bromine, iodine, trifluoroacetyl, 6-morpholinopyridin-3-yl, —NH₂, methoxy, ethoxy, propoxy, butoxy, pentoxy, hexoxy, or heptoxy;
R₄ is the same or different and is selected from hydrogen, hydroxy, alkyl, methyl, ethyl, halogen, —N(CH₃)₂, alkoxy, heteroalkyl, or —OR₁₂ where R₁₂ is substituted or unsubstituted alkyl or heteroalkyl, substituted or unsubstituted alkenyl or heteroalkenyl, or substituted or unsubstituted alkynyl or heteroalkynyl. Exemplary R₄ is hydrogen, hydroxyl, methyl, fluorine, chlorine, bromine, iodine, —N(CH₃)₂, methoxy, ethoxy, propoxy, butoxy, pentoxy, hexoxy, heptoxy, morpholinomethyl, 2-oxy-3,4,5-trihydroxy-6-(hydroxymethyl)tetrahydro-2H-pyran-2-yl, or N-(2-(3,4,5-trihydroxy-6-(hydroxymethyl)tetrahydro-2H-pyran-2-yloxy)ethyl)acetamido;
   R₅ is hydrogen or hydroxy;
   R₆ is oxygen, sulfur, NH or CH₂;
   R₈ is hydrogen, halogen, fluorine, chlorine, bromine, iodine, carboxylate, carboxylate alkyl ester, alkoxycarbonyl or methoxycarbonyl;
   R₉ is hydrogen, halogen, fluorine, chlorine, bromine, iodine, carboxylate, carboxylate alkyl ester, alkoxycarbonyl or methoxycarbonyl;
   R₁₀ is hydrogen or R₁₀ is connected to R₃ wherein R₁₀—X—R₃ is S—C═N or N═C—S;
   R₂₃ is hydrogen, 2-bromoethyl, 2(N,N-dimethylamino)ethyl, 2(N,N-diethylamino)ethyl, 2-(pyrrolidin-1-yl)ethyl, 2-morpholinoethyl, 2-(piperazin-1-yl)ethyl or 2-(4-methylpiperazin-1-yl)ethyl.

One of skill will readily understand based on the present disclosure to select a moiety from among the species of each of R₁, R₂, R₃, R₄, R₅, R₆, R₈, R₉, R₁₀ and R₂₃ to arrive at compounds of the present disclosure. One of skill will also readily understand and envision that subsets of the species of one or more of R₁, R₂, R₃, R₄, R₅, R₆, R₈, R₉, R₁₀ and R₂₃ are contemplated, and that one of skill will readily be able to identify subsets of species of one or more of R₁, R₂, R₃, R₄, R₅, R₆, R₈, R₉, R₁₀ and R₂₃ as desired to produce alternate compound formulae of various scope. Stated differently, the present specification describes each of the species represented by the Formula VI with all of the various combinations of the various moieties for each of R₁, R₂, R₃, R₄, R₅, R₆, R₈, R₉, R₁₀ and R₂₃ without expressly reciting each species individually. Stated differently, compounds within the scope of Formula VI include those being a subset of the combinations of the various moieties for each of R₁, R₂, R₃, R₄, R₅, R₆, R₈, R₉, R₁₀ and R₂₃. Likewise, one of ordinary skill in the art will readily understand that a particular moiety or moieties can be deselected from among those various moieties for each of R₁, R₂, R₃, R₄, R₅, R₆, R₈, R₉, R₁₀ and R₂₃. Likewise, one of skill will readily understand that compounds within the scope of the present disclosure can be described using “proviso” language, for example with the proviso that for any particular R group, R is not one or more of the recited species for the R group. For example, compounds include those within the scope of Formula VI as described above with the proviso that each of R₁, R₂, R₃, R₄, R₅, R₈, R₉, and R₁₀ are not hydrogen when R₆ is O. For example, compounds include those within the scope of Formula VI as described above with the proviso that the compound is not compound 1181, 1282, 1289 or 1287.

Certain compounds within the scope of the present disclosure include those of Formula VII.

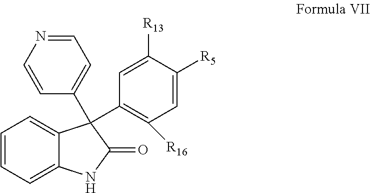

As shown in Formula VII, R₁₃ is hydrogen, fluorine, chlorine, bromine, iodine, methyl, ethyl, propyl, n-propyl, isopropyl, butyl, n-butyl, isobutyl, tert-butyl, sec-butyl, pentyl, n-pentyl, cyclopentyl, hexyl, n-hexyl, cyclohexyl, heptyl, n-heptyl, cycloheptyl, methoxy, ethoxy, propoxy, butoxy, pentoxy, hexoxy, heptoxy or 2-oxy-3,4,5-trihydroxy-6-(hydroxymethyl)tetahydro-2H-pyran-2-yl.

R₁₆ is hydrogen, hydroxy, alkoxy, substituted alkoxy, methoxy, —O—CH₂—R₁₂, ethoxy, —O—CH₂—CH₂—R₂₁, propargyloxy, 1H-1,2,3-triazol-1-yl, 4-(pyridin-2-ylmethyl)-1H-1,2,3-triazol-1-yl, 4-((5-(2-hydroxyethylamino)pyridin-2-yl)methyl)-1H-1,2,3-triazol-1-yl) wherein R₁₂ is 1H-1,2,3-triazol-4-yl, 1-(2-morpholinoethyl)-1H-1,2,3-triazol-4-yl, carboxymethylene, 2-acetamide, N-(2-hydroxyethyl)-2-acetamido, N-(2-(3,4,5-trihydroxy-6-(hydroxymethyl)tetrahydro-2H-pyran-2-yloxy)ethyl)acetamido, wherein R₂₁ is hydroxy, methoxy, amino, N-methylamino, N,N-dimethylamino, N,N-diethylamino, [N-methyl,N-ethyl]amino, bromo, acetoxy, N-morpholino, N-pyrrolidino, N-piperidino, N-piperazino, N-[4-methyl]-piperazino, ((3R,4S,5S,6R)-2-oxy-3,4,5-trihydroxy-6-(hydroxymethyl)tetrahydro-2H-pyran-2-yl) or 2-oxy-3,4,5-trihydroxy-6-(hydroxymethyl)tetrahydro-2H-pyran-2-yl). Exemplary R₁₆ is hydrogen, hydroxy, methoxy, substituted methoxy, ethoxy, substituted ethoxy, N-(2-ethoxy)-morpholino, 2-(N,N-dimethylamino)ethoxy, N-(2-ethoxy)-piperazin-1-yl, (1-(N-2-ethyl-morpholino)-1H-1,2,3-triazol-5-yl)methoxy, 4-((5-((2-hydroxyethyl)amino)pyridin-2-yl)methyl)-1H-1,2,3-triazol-1-yl, 4-(pyridin-2-ylmethyl)-1H-1,2,3-triazol-1-yl, prop-2-ynyloxy, N-(3,4,5-trihydroxy-6-(hydroxymethyl)tetrahydro-2H-pyran-2-yl)oxy)ethyl)-2-oxyacetamido, 2-oxyacetic acid, 2-oxyethanol, 2-methoxyethoxy, 2-acetoxyethyl, 2-bromoethoxy, 2-acetoxyethoxy, ethyl 2-oxyacetate, 2-(N,N-dimethylamino)ethoxy, 2-(N,N-diethylamino)ethoxy, 2-(pyrrolidin-1-yl)ethoxy, 2-(piperidin-1-yl)ethoxy, 2-morpholinoethoxy, 2-(piperazin-1-yl)ethoxy, or 2-(4-methylpiperazin-1-yl)ethoxy.

R₅ is hydrogen or hydroxy.

One of skill will readily understand based on the present disclosure to select a moiety from among the species of each of R₁₃, R₁₆, and R₅ to arrive at compounds of the present disclosure. One of skill will also readily understand and envision that subsets of the species of one or more of R₁₃, R₁₆, and R₅ are contemplated, and that one of skill will readily be able to identify subsets of species of one or more of R₁₃, R₁₆, and R₅ as desired to produce alternate compound formulae of various scope. The present specification describes each of the species represented by the Formula VII with all of the various combinations of the various moieties for each of R₁₃, R₁₆, and R₅ without expressly reciting each species individually. Compounds within the scope of Formula VII include those being a subset of the combinations of the various moieties for each of R₁₃, R₁₆, and R₅. Likewise, one of ordinary skill in the art will readily understand that a particular moiety or moieties can be deselected from among those various moieties for each of R₁₃, R₁₆, and R₅. Likewise, one of skill will readily understand that compounds within the scope of the present disclosure can be described using “proviso” language, for example with the proviso that for any particular R group, R is not one or more of the recited species for the R group. For example, compounds include those within the scope of Formula VII as described above with the proviso that each of R₁₃, R₁₆, and R₁₇ are not hydrogen. For example, compounds include those within the scope of Formula VII as described above with the proviso that the compound is not compound 1181, 1282, 1289 or 1287.

Certain compounds within the scope of the present disclosure include those of Formula VIII.

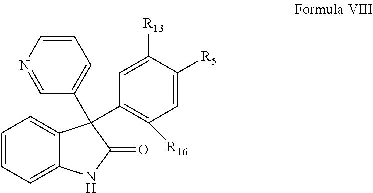

As shown in Formula VIII, R₁₃ is hydrogen, fluorine, chlorine, bromine, iodine, methyl, ethyl, propyl, n-propyl, isopropyl, butyl, n-butyl, isobutyl, tert-butyl, sec-butyl, pentyl, n-pentyl, cyclopentyl, hexyl, n-hexyl, cyclohexyl, heptyl, n-heptyl, cycloheptyl, methoxy, ethoxy, propoxy, butoxy, pentoxy, hexoxy, heptoxy or 2-oxy-3,4,5-trihydroxy-6-(hydroxymethyl)tetahydro-2H-pyran-2-yl.

R₁₆ is hydrogen, hydroxy, alkoxy, substituted alkoxy, methoxy, —O—CH₂—R₁₂, ethoxy, —O—CH₂—CH₂—R₂₁, propargyloxy, 1H-1,2,3-triazol-1-yl, 4-(pyridin-2-ylmethyl)-1H-1,2,3-triazol-1-yl, 4-((5-(2-hydroxyethylamino)pyridin-2-yl)methyl)-1H-1,2,3-triazol-1-yl) wherein R₁₂ is 1H-1,2,3-triazol-4-yl, 1-(2-morpholinoethyl)-1H-1,2,3-triazol-4-yl, carboxymethylene, 2-acetamide, N-(2-hydroxyethyl)-2-acetamido, N-(2-(3,4,5-trihydroxy-6-(hydroxymethyl)tetrahydro-2H-pyran-2-yloxy)ethyl)acetamido, wherein R₂₁ is hydroxy, methoxy, amino, N-methylamino, N,N-dimethylamino, N,N-diethylamino, [N-methyl,N-ethyl]amino, bromo, acetoxy, N-morpholino, N-pyrrolidino, N-piperidino, N-piperazino, N-[4-methyl]-piperazino, ((3R,4S,5S,6R)-2-oxy-3,4,5-trihydroxy-6-(hydroxymethyl)tetrahydro-2H-pyran-2-yl) or 2-oxy-3,4,5-trihydroxy-6-(hydroxymethyl)tetrahydro-2H-pyran-2-yl). Exemplary R₁₆ is hydrogen, hydroxy, methoxy, substituted methoxy, ethoxy, substituted ethoxy, N-(2-ethoxy)-morpholino, 2-(N,N-dimethylamino)ethoxy, N-(2-ethoxy)-piperazin-1-yl, (1-(N-2-ethyl-morpholino)-1H-1,2,3-triazol-5-yl)methoxy, 4-((5-((2-hydroxyethyl)amino)pyridin-2-yl)methyl)-1H-1,2,3-triazol-1-yl, 4-(pyridin-2-ylmethyl)-1H-1,2,3-triazol-1-yl, prop-2-ynyloxy, N-(3,4,5-trihydroxy-6-(hydroxymethyl)tetrahydro-2H-pyran-2-yl)oxy)ethyl)-2-oxyacetamido, 2-oxyacetic acid, 2-oxyethanol, 2-methoxyethoxy, 2-acetoxyethyl, 2-bromoethoxy, 2-acetoxyethoxy, ethyl 2-oxyacetate, 2-(N,N-dimethylamino)ethoxy, 2-(N,N-diethylamino)ethoxy, 2-(pyrrolidin-1-yl)ethoxy, 2-(piperidin-1-yl)ethoxy, 2-morpholinoethoxy, 2-(piperazin-1-yl)ethoxy, or 2-(4-methylpiperazin-1-yl)ethoxy.

R₅ is hydrogen or hydroxy.

One of skill will readily understand based on the present disclosure to select a moiety from among the species of each of R₁₃, R₁₆, and R₅ to arrive at compounds of the present disclosure. One of skill will also readily understand and envision that subsets of the species of one or more of R₁₃, R₁₆, and R₅ are contemplated, and that one of skill will readily be able to identify subsets of species of one or more of R₁₃, R₁₆, and R₅ as desired to produce alternate compound formulae of various scope. The present specification describes each of the species represented by the Formula VIII with all of the various combinations of the various moieties for each of R₁₃, R₁₆, and R₅ without expressly reciting each species individually. Compounds within the scope of Formula VIII include those being a subset of the combinations of the various moieties for each of R₁₃, R₁₆, and R₅. Likewise, one of ordinary skill in the art will readily understand that a particular moiety or moieties can be deselected from among those various moieties for each of R₁₃, R₁₆, and R₅. Likewise, one of skill will readily understand that compounds within the scope of the present disclosure can be described using “proviso” language, for example with the proviso that for any particular R group, R is not one or more of the recited species for the R group. For example, compounds include those within the scope of Formula VIII as described above with the proviso that each of R₁₃, R₁₆, and R₁₇ are not hydrogen. For example, compounds include those within the scope of Formula VIII as described above with the proviso that the compound is not compound 1181, 1282, 1289 or 1287.

Exemplary compounds include the following:

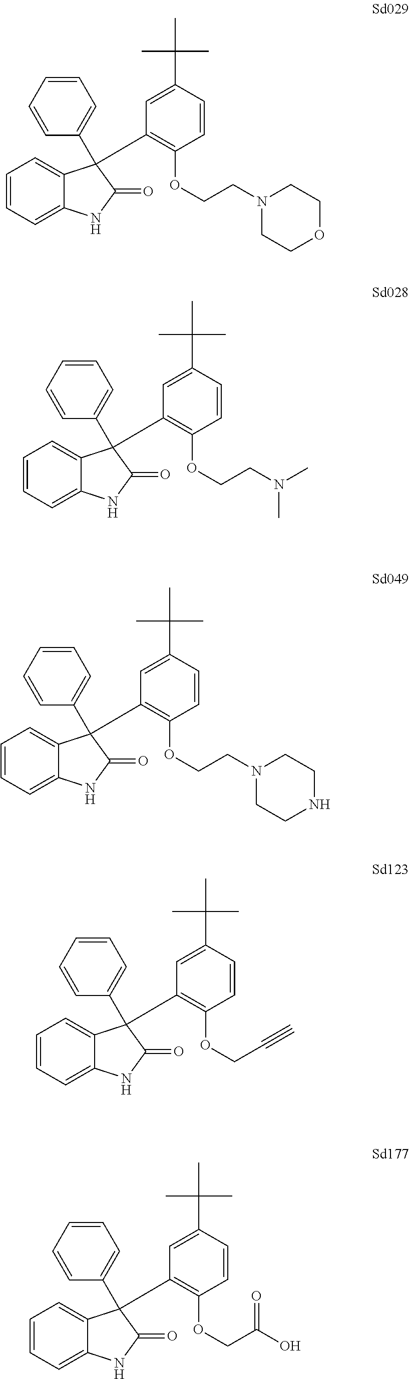 [SMILES: CC(C)(C)c1ccc(OCCN2CCOCC2)c(C2(c3ccccc3)C(=O)Nc3ccccc32)c1 ; CN(C)CCOc1ccc(C(C)(C)C)cc1C1(c2ccccc2)C(=O)Nc2ccccc21 ; CC(C)(C)c1ccc(OCCN2CCNCC2)c(C2(c3ccccc3)C(=O)Nc3ccccc32)c1 ; C#CCOc1ccc(C(C)(C)C)cc1C1(c2ccccc2)C(=O)Nc2ccccc21 ; CC(C)(C)c1ccc(OCC(=O)O)c(C2(c3ccccc3)C(=O)Nc3ccccc32)c1]

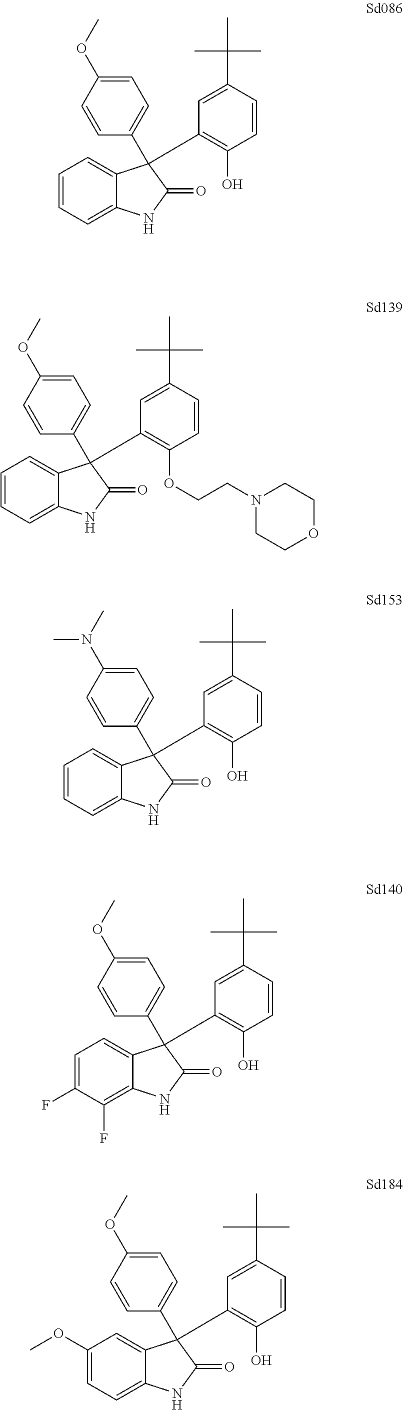

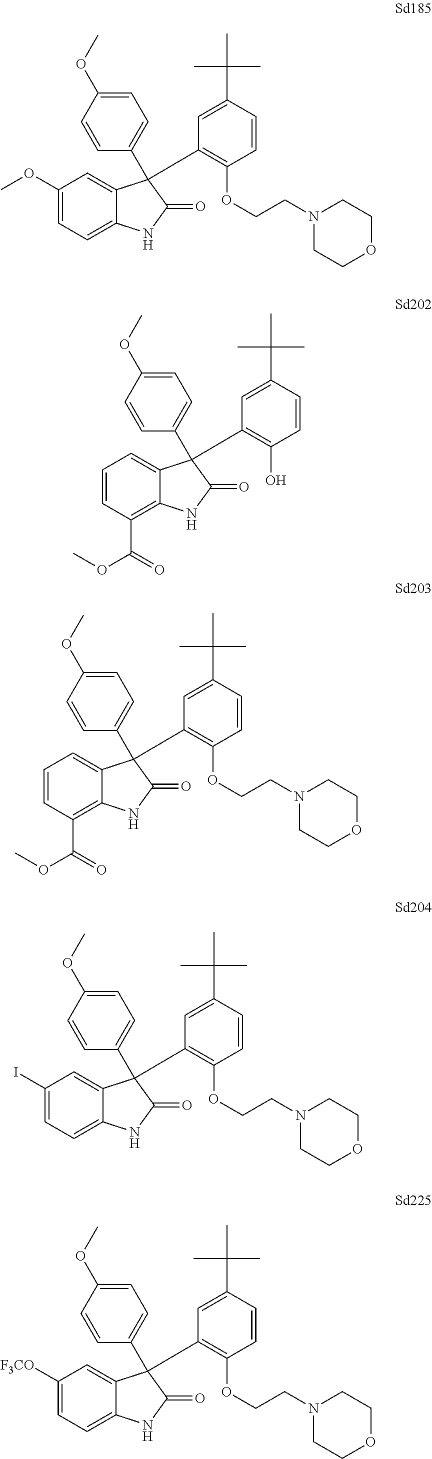

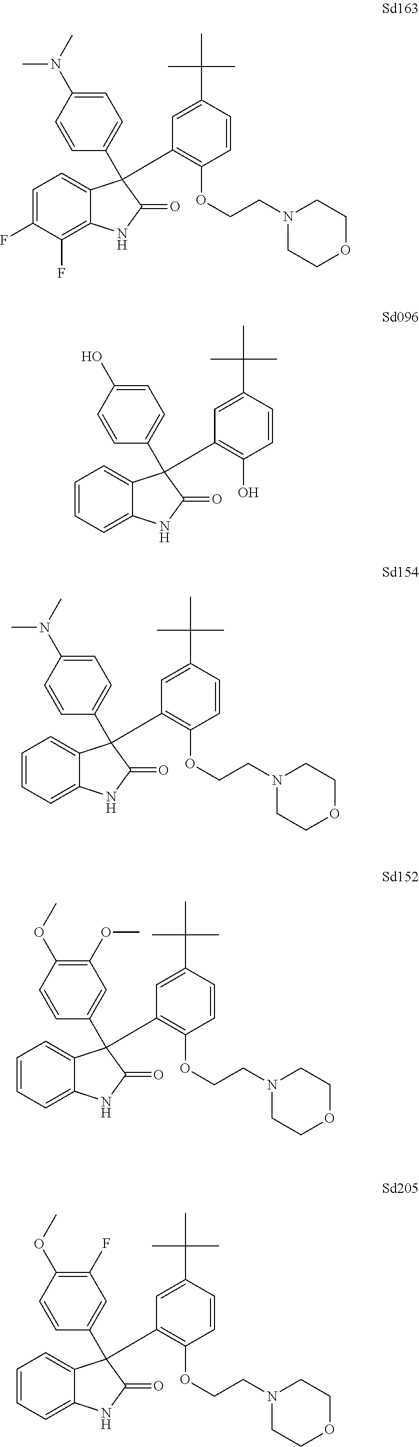 [SMILES: CN(C)c1ccc(C2(c3cc(C(C)(C)C)ccc3OCCN3CCOCC3)C(=O)Nc3c2ccc(F)c3F)cc1 ; CC(C)(C)c1ccc(O)c(C2(c3ccc(O)cc3)C(=O)Nc3ccccc32)c1 ; CN(C)c1ccc(C2(c3cc(C(C)(C)C)ccc3OCCN3CCOCC3)C(=O)Nc3ccccc32)cc1 ; COc1ccc(C2(c3cc(C(C)(C)C)ccc3OCCN3CCOCC3)C(=O)Nc3ccccc32)cc1OC ; COc1ccc(C2(c3cc(C(C)(C)C)ccc3OCCN3CCOCC3)C(=O)Nc3ccccc32)cc1F]

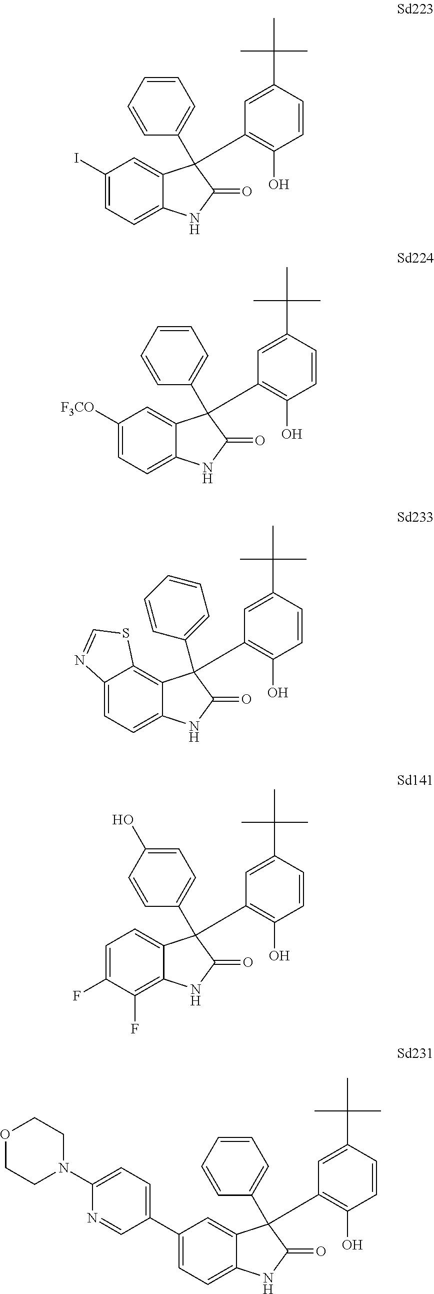 [SMILES: CC(C)(C)c1ccc(O)c(C2(c3ccccc3)C(=O)Nc3ccc(I)cc32)c1 ; CC(C)(C)c1ccc(O)c(C2(c3ccccc3)C(=O)Nc3ccc(OC(F)(F)F)cc32)c1 ; CC(C)(C)c1ccc(O)c(C2(c3ccccc3)C(=O)Nc3ccc4ncsc4c32)c1 ; CC(C)(C)c1ccc(O)c(C2(c3ccc(O)cc3)C(=O)Nc3c2ccc(F)c3F)c1 ; CC(C)(C)c1ccc(O)c(C2(c3ccccc3)C(=O)Nc3ccc(-c4ccc(N5CCOCC5)nc4)cc32)c1]

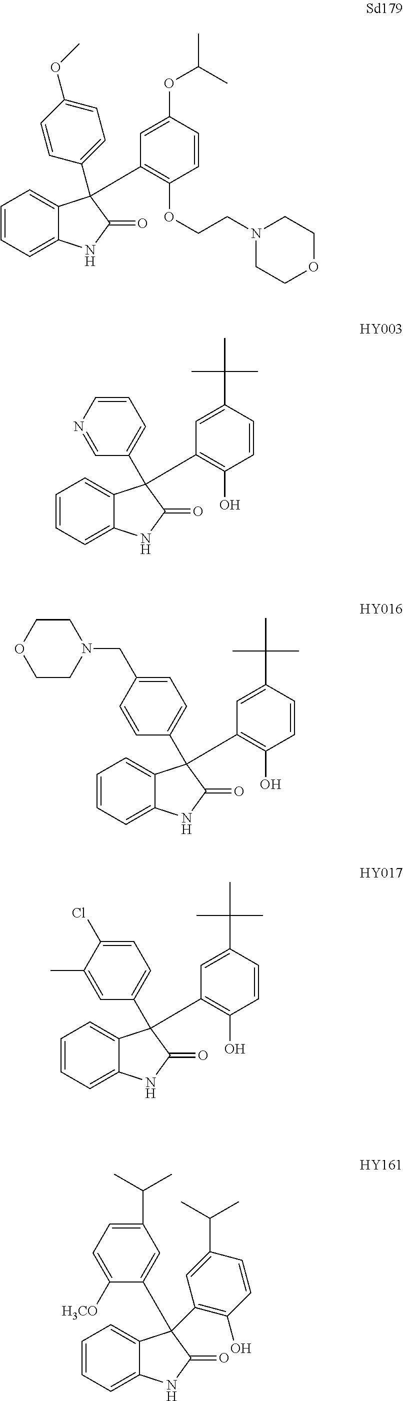

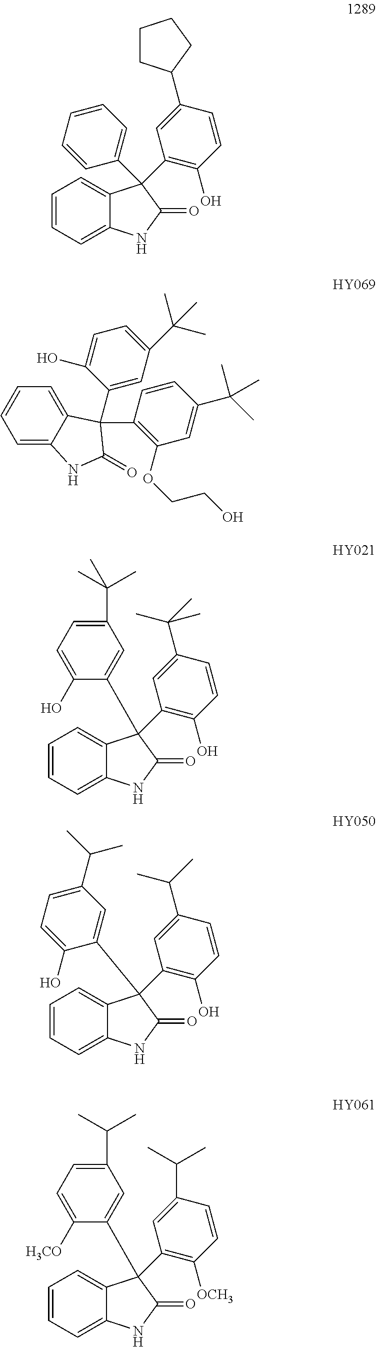

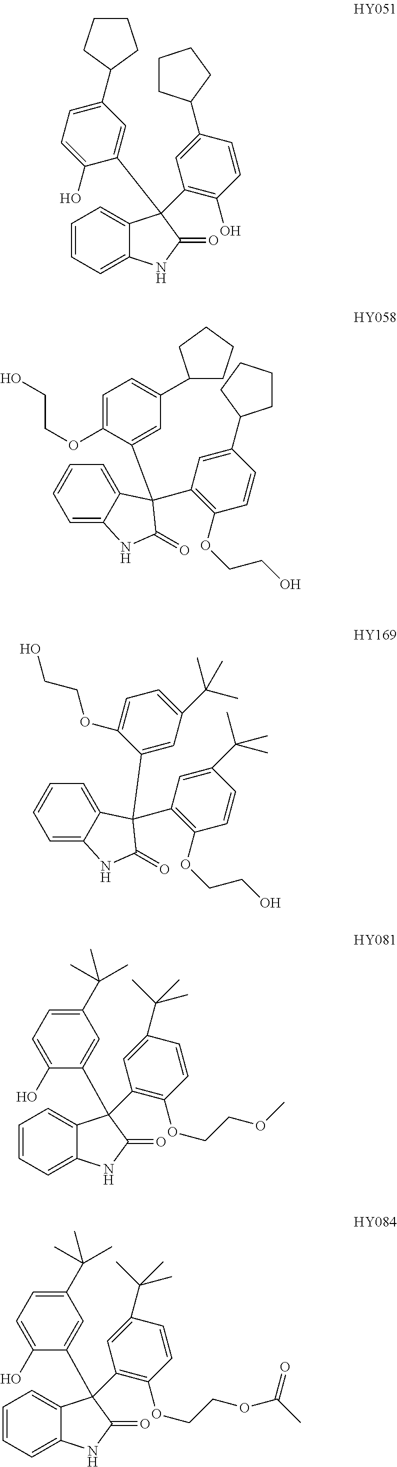 [SMILES: O=C1Nc2ccccc2C1(c1cc(C2CCCC2)ccc1O)c1cc(C2CCCC2)ccc1O ; O=C1Nc2ccccc2C1(c1cc(C2CCCC2)ccc1OCCO)c1cc(C2CCCC2)ccc1OCCO ; CC(C)(C)c1ccc(OCCO)c(C2(c3cc(C(C)(C)C)ccc3OCCO)C(=O)Nc3ccccc32)c1 ; COCCOc1ccc(C(C)(C)C)cc1C1(c2cc(C(C)(C)C)ccc2O)C(=O)Nc2ccccc21 ; CC(=O)OCCOc1ccc(C(C)(C)C)cc1C1(c2cc(C(C)(C)C)ccc2O)C(=O)Nc2ccccc21]

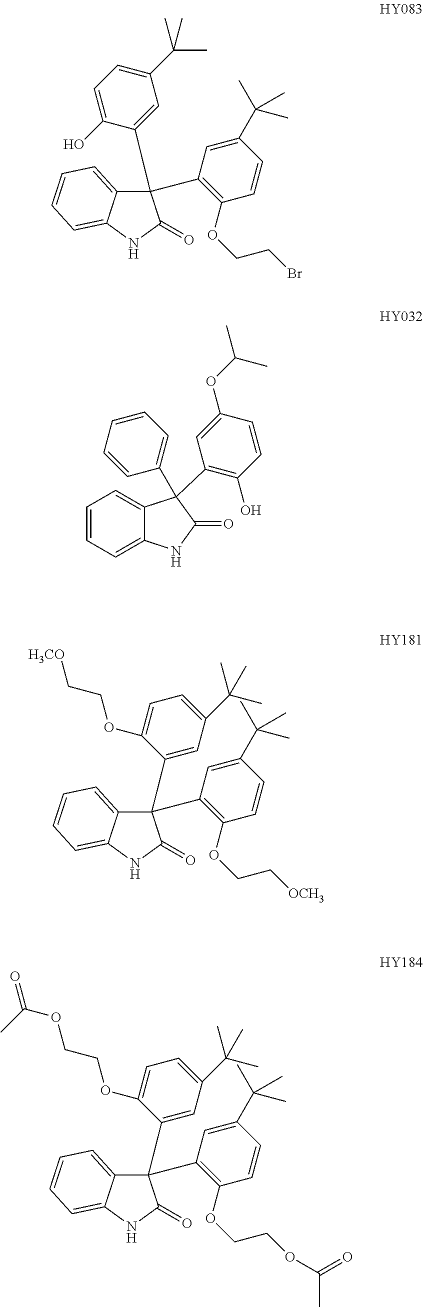 [SMILES: CC(C)(C)c1ccc(O)c(C2(c3cc(C(C)(C)C)ccc3OCCBr)C(=O)Nc3ccccc32)c1 ; CC(C)Oc1ccc(O)c(C2(c3ccccc3)C(=O)Nc3ccccc32)c1 ; COCCOc1ccc(C(C)(C)C)cc1C1(c2cc(C(C)(C)C)ccc2OCCOC)C(=O)Nc2ccccc21 ; CC(=O)OCCOc1ccc(C(C)(C)C)cc1C1(c2cc(C(C)(C)C)ccc2OCCOC(C)=O)C(=O)Nc2ccccc21]

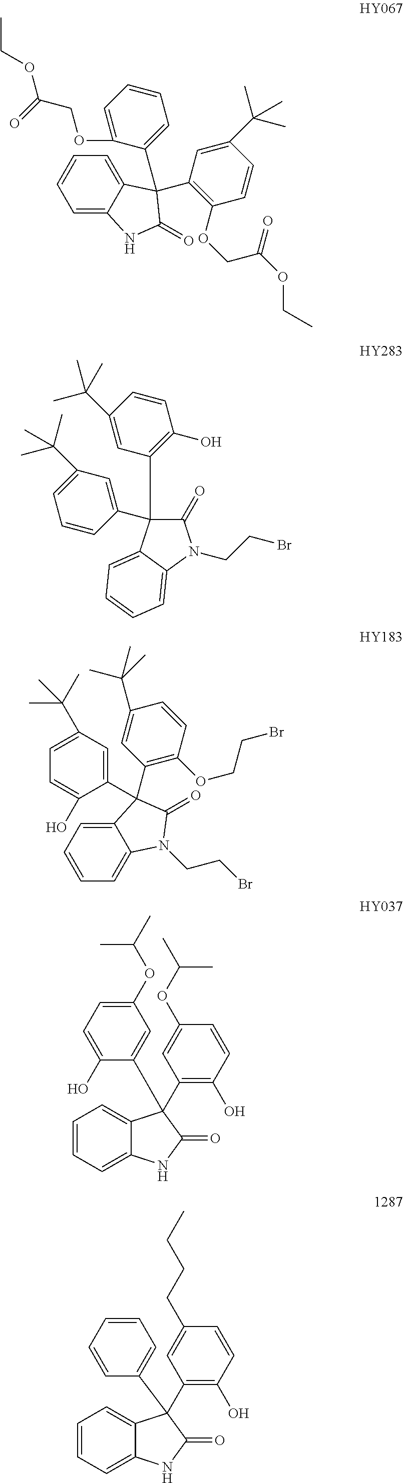

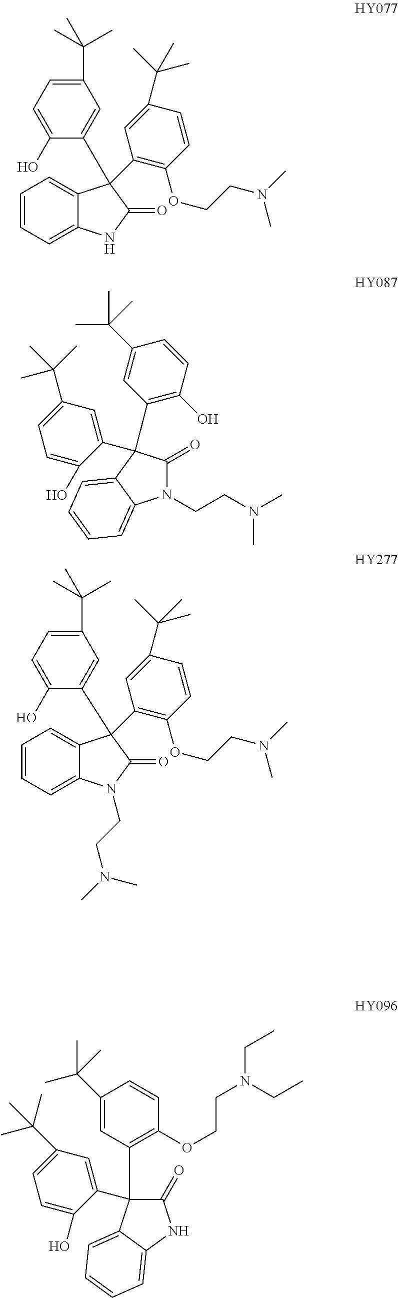

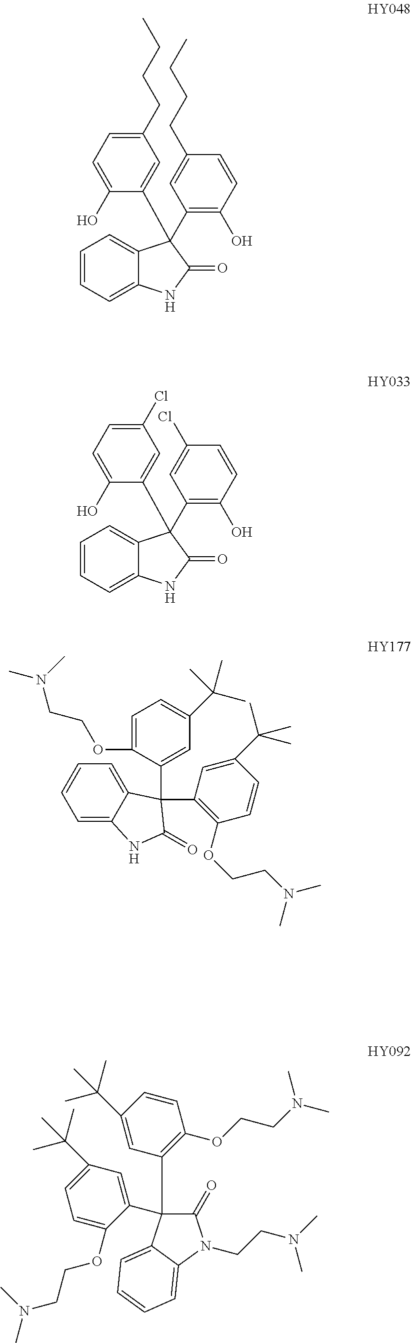

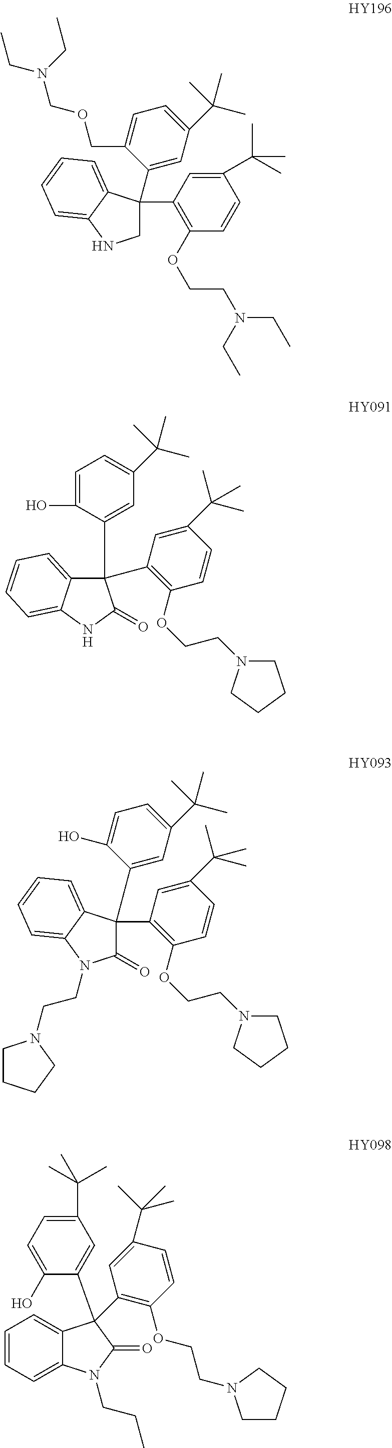 [SMILES: CCN(CC)CCOc1ccc(C(C)(C)C)cc1C1(c2cc(C(C)(C)C)ccc2COCN(CC)CC)CNc2ccccc21 ; CC(C)(C)c1ccc(O)c(C2(c3cc(C(C)(C)C)ccc3OCCN3CCCC3)C(=O)Nc3ccccc32)c1 ; CC(C)(C)c1ccc(O)c(C2(c3cc(C(C)(C)C)ccc3OCCN3CCCC3)C(=O)N(CCN3CCCC3)c3ccccc32)c1 ; CCCN1C(=O)C(c2cc(C(C)(C)C)ccc2O)(c2cc(C(C)(C)C)ccc2OCCN2CCCC2)c2ccccc21]

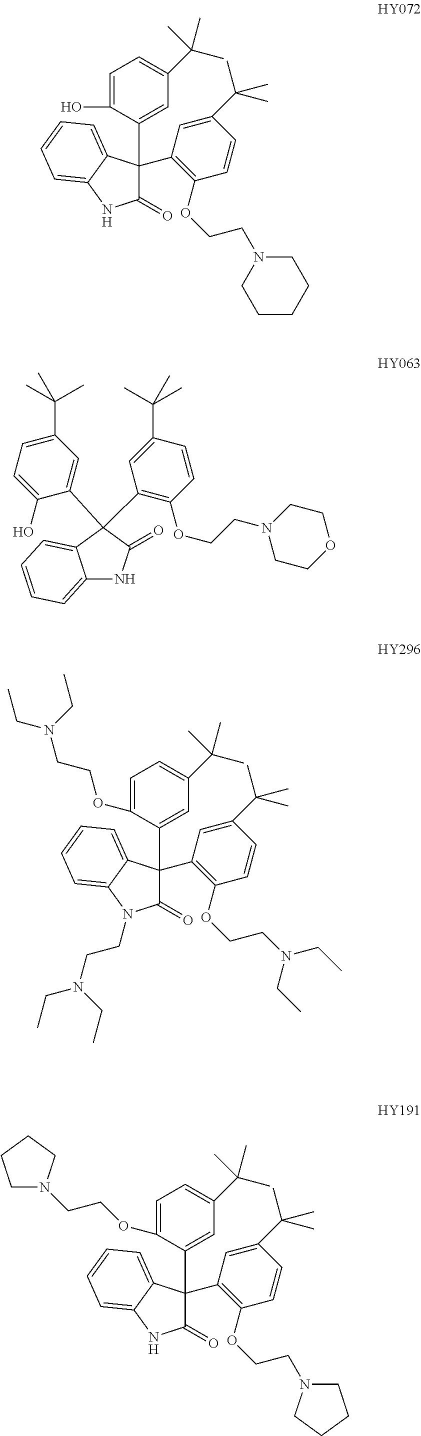 [SMILES: CC(C)(C)c1ccc(O)c(C2(c3cc(C(C)(C)C)ccc3OCCN3CCCCC3)C(=O)Nc3ccccc32)c1 ; CC(C)(C)c1ccc(O)c(C2(c3cc(C(C)(C)C)ccc3OCCN3CCOCC3)C(=O)Nc3ccccc32)c1 ; CCN(CC)CCOc1ccc(C(C)(C)C)cc1C1(c2cc(C(C)(C)C)ccc2OCCN(CC)CC)C(=O)N(CCN(CC)CC)c2ccccc21 ; CC(C)(C)c1ccc(OCCN2CCCC2)c(C2(c3cc(C(C)(C)C)ccc3OCCN3CCCC3)C(=O)Nc3ccccc32)c1]

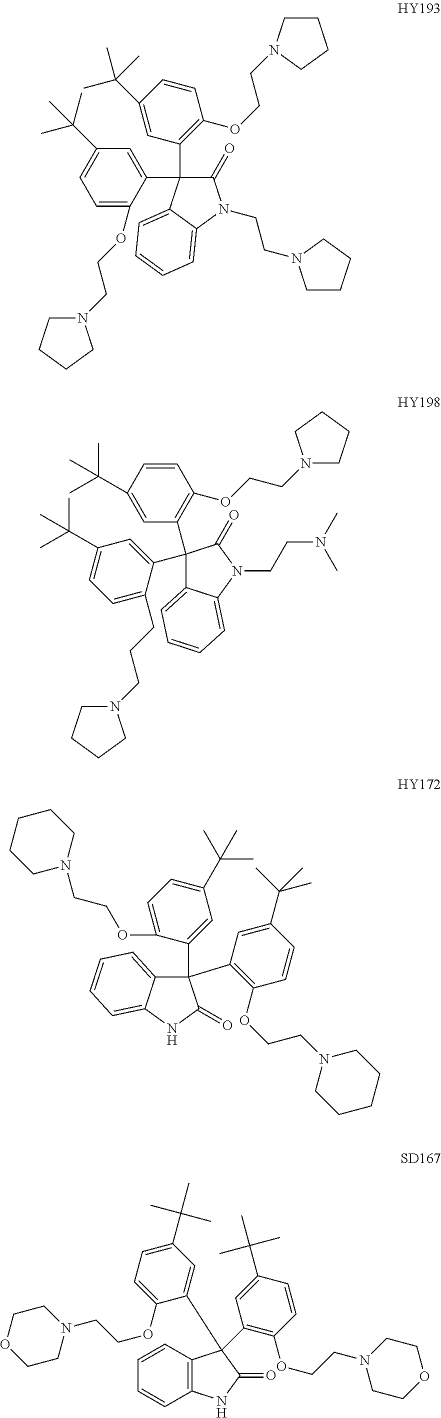 [SMILES: CC(C)(C)c1ccc(OCCN2CCCC2)c(C2(c3cc(C(C)(C)C)ccc3OCCN3CCCC3)C(=O)N(CCN3CCCC3)c3ccccc32)c1 ; CN(C)CCN1C(=O)C(c2cc(C(C)(C)C)ccc2CCCN2CCCC2)(c2cc(C(C)(C)C)ccc2OCCN2CCCC2)c2ccccc21 ; CC(C)(C)c1ccc(OCCN2CCCCC2)c(C2(c3cc(C(C)(C)C)ccc3OCCN3CCCCC3)C(=O)Nc3ccccc32)c1 ; CC(C)(C)c1ccc(OCCN2CCOCC2)c(C2(c3cc(C(C)(C)C)ccc3OCCN3CCOCC3)C(=O)Nc3ccccc32)c1]

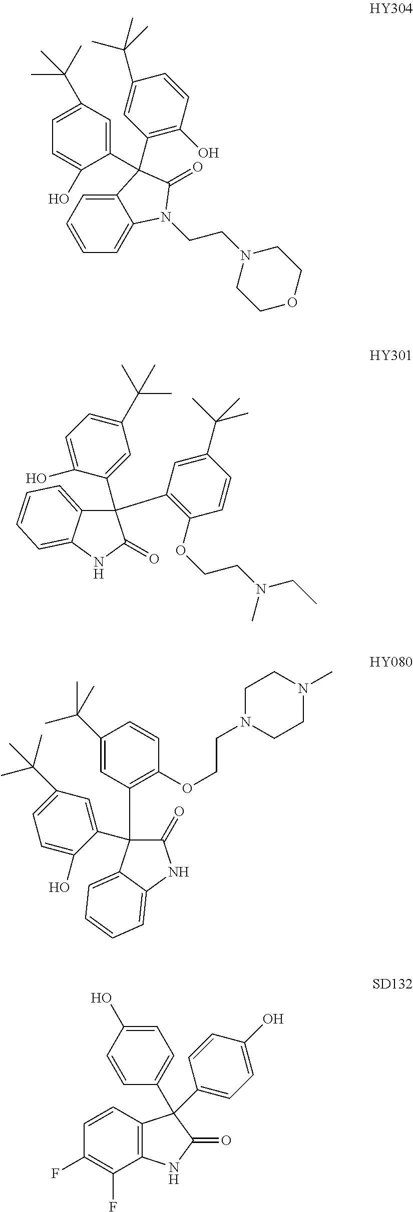

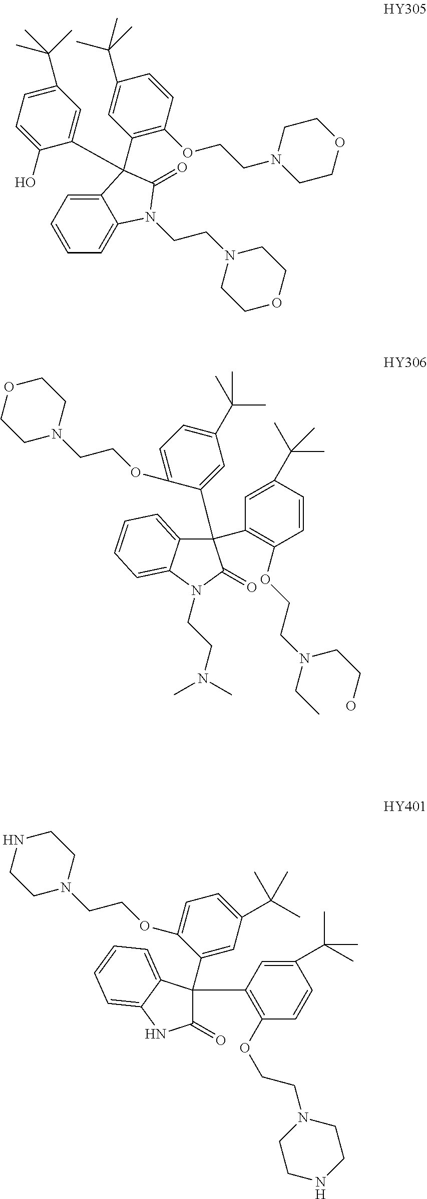 [SMILES: CC(C)(C)c1ccc(O)c(C2(c3cc(C(C)(C)C)ccc3OCCN3CCOCC3)C(=O)N(CCN3CCOCC3)c3ccccc32)c1 ; CCN(CCO)CCOc1ccc(C(C)(C)C)cc1C1(c2cc(C(C)(C)C)ccc2OCCN2CCOCC2)C(=O)N(CCN(C)C)c2ccccc21 ; CC(C)(C)c1ccc(OCCN2CCNCC2)c(C2(c3cc(C(C)(C)C)ccc3OCCN3CCNCC3)C(=O)Nc3ccccc32)c1]

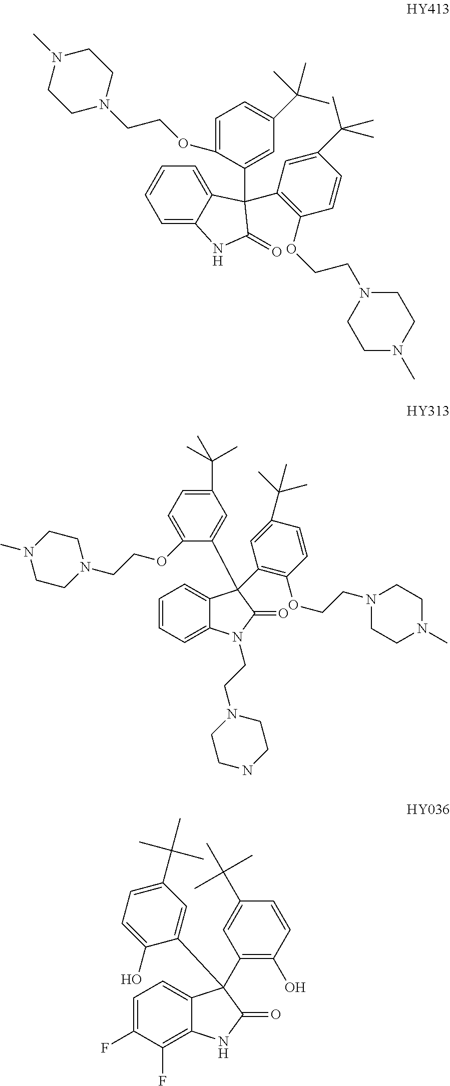

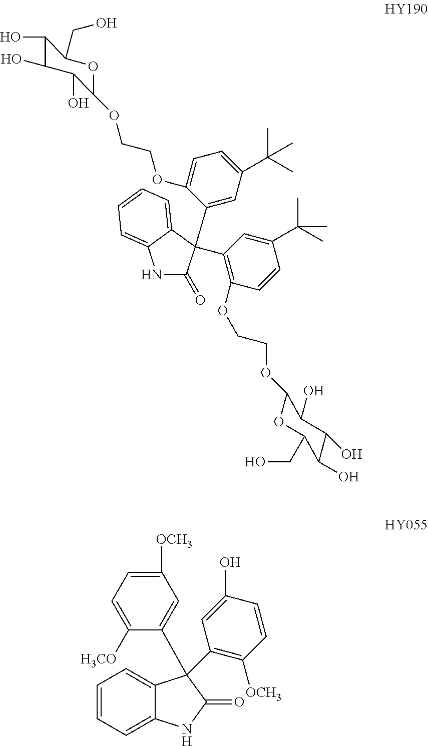 [SMILES: CC(C)(C)c1ccc(OCCOC2OC(CO)C(O)C(O)C2O)c(C2(c3cc(C(C)(C)C)ccc3OCCOC3OC(CO)C(O)C(O)C3O)C(=O)Nc3ccccc32)c1 ; COc1ccc(OC)c(C2(c3cc(O)ccc3OC)C(=O)Nc3ccccc32)c1]

In at least certain examples, the compounds disclosed here can be used in the treatment of cellular proliferative disorders, such as cancer, and noncancerous proliferative disorders. Treatment of cellular proliferative disorders is intended to include, but is not limited to, inhibition of proliferation including rapid proliferation. As used herein, the term “cellular proliferative disorder” includes, but is not limited to, disorders characterized by undesirable or inappropriate proliferation of one or more subset(s) of cells in a multicellular organism. The term “cancer” refers to various types of malignant neoplasms, most of which can invade surrounding tissues, and may metastasize to different sites (see, for example, PDR Medical Dictionary 1st edition (1995), incorporated herein by reference in its entirety for all purposes). The terms “neoplasm” and “tumor” refer to an abnormal tissue that grows by cellular proliferation more rapidly than normal and continues to grow after the stimuli that initiated proliferation is removed. Id. Such abnormal tissue shows partial or complete lack of structural organization and functional coordination with the normal tissue which may be either benign (i.e., benign tumor) or malignant (i.e., malignant tumor).

The language “treatment of cellular proliferative disorders” is intended to include, but is not limited to, the prevention of the growth of neoplasms in a subject or a reduction in the growth of pre-existing neoplasms in a subject. The inhibition also can be the inhibition of the metastasis of a neoplasm from one site to another. In certain embodiments, the neoplasms are sensitive to one or more diaryloxindole compounds of the present invention. Examples of the types of neoplasms intended to be encompassed by the present invention include, but are not limited to, those neoplasms associated with cancers of the breast, skin, bone, prostate, ovaries, uterus, cervix, liver, lung, brain, larynx, gallbladder, pancreas, rectum, parathyroid, thyroid, adrenal gland, immune system, neural tissue, head and neck, colon, stomach, bronchi, and/or kidneys.

Examples of general categories of cancer include, but are not limited to, carcinomas (i.e., malignant tumors derived from epithelial cells such as, for example, common forms of breast, prostate, lung and colon cancer), sarcomas (i.e., malignant tumors derived from connective tissue or mesenchymal cells), lymphomas (i.e., malignancies derived from hematopoietic cells), leukemias (i.e., malignancies derived from hematopoietic cells), germ cell tumors (i.e., tumors derived from totipotent cells. In adults most often found in the testicle or ovary; in fetuses, babies and young children, most often found on the body midline, particularly at the tip of the tailbone), blastic tumors (i.e., a typically malignant tumor which resembles an immature or embryonic tissue) and the like.

Examples of specific neoplasms intended to be encompassed by the present invention include, but are not limited to, acute lymphoblastic leukemia; myeloid leukemia, acute myeloid leukemia, childhood; adrenocortical carcinoma; AIDS-related cancers; AIDS-related lymphoma; anal cancer; appendix cancer; astrocytoma (e.g., cerebellar, cerebral); atypical teratoid/rhabdoid tumor; basal cell carcinoma; bile duct cancer, extrahepatic; bladder cancer; bone cancer, osteosarcoma and malignant fibrous histiocytoma; brain tumor (e.g., brain stem glioma, central nervous system atypical teratoid/rhabdoid tumors, central nervous system embryonal tumors, cerebellar astrocytoma, cerebral astrocytoma/malignant glioma, craniopharyngioma, ependymoblastoma, ependymoma, medulloblastoma, medulloepithelioma, pineal parenchymal tumors of intermediate differentiation, supratentorial primitive neuroectodermal tumors and/or pineoblastoma, visual pathway and/or hypothalamic glioma, brain and spinal cord tumors); breast cancer; bronchial tumors; Burkitt lymphoma; carcinoid tumor (e.g., gastrointestinal); carcinoma of unknown primary; central nervous system (e.g., atypical teratoid/rhabdoid tumor, embryonal tumors (e.g., lymphoma, primary); cerebellar astrocytoma; cerebral astrocytoma/malignant glioma; cervical cancer; chordoma; chronic lymphocytic leukemia; chronic myelogenous leukemia; chronic myeloproliferative disorders; colon cancer; colorectal cancer; craniopharyngioma; cutaneous T-cell lymphoma; embryonal tumors, central nervous system; endometrial cancer; ependymoblastoma; ependymoma; esophageal cancer; Ewing family of tumors; extracranial germ cell tumor; extragonadal germ cell tumor; extrahepatic bile duct cancer; eye cancer (e.g., intraocular melanoma, retinoblastoma); gallbladder cancer; gastric cancer; gastrointestinal tumor (e.g., carcinoid tumor, stromal tumor (gist), stromal cell tumor); germ cell tumor (e.g., extracranial, extragonadal, ovarian); gestational trophoblastic tumor; glioma (e.g., brain stem, cerebral astrocytoma); hairy cell leukemia; head and neck cancer; hepatocellular cancer; Hodgkin lymphoma; hypopharyngeal cancer; hypothalamic and visual pathway glioma; intraocular melanoma; islet cell tumors; Kaposi sarcoma; kidney cancer; large cell tumors; laryngeal cancer (e.g., acute lymphoblastic, acute myeloid); leukemia (e.g., acute myeloid, chronic lymphocytic, chronic myelogenous, hairy cell); lip and/or oral cavity cancer; liver cancer; lung cancer (e.g., non-small cell, small cell); lymphoma (e.g., AIDS-related, Burkitt, cutaneous Tcell, Hodgkin, non-Hodgkin, primary central nervous system); macroglobulinemia, Waldenström; malignant fibrous histiocytoma of bone and/or osteosarcoma; medulloblastoma; medulloepithelioma; melanoma; merkel cell carcinoma; mesothelioma; metastatic squamous neck cancer; mouth cancer; multiple endocrine neoplasia syndrome; multiple myeloma/plasma cell neoplasm; mycosis fungoides; myelodysplastic syndromes; myelodysplastic/myeloproliferative diseases; myelogenous leukemia (e.g., chronic, acute, multiple); myeloproliferative disorders, chronic; nasal cavity and/or paranasal sinus cancer; nasopharyngeal cancer; neuroblastoma; non-Hodgkin lymphoma; non-small cell lung cancer; oral cancer; oral cavity cancer, oropharyngeal cancer; osteosarcoma and/or malignant fibrous histiocytoma of bone; ovarian cancer (e.g., ovarian epithelial cancer, ovarian germ cell tumor, ovarian low malignant potential tumor); pancreatic cancer (e.g., islet cell tumors); papillomatosis; paranasal sinus and/or nasal cavity cancer; parathyroid cancer; penile cancer; pharyngeal cancer; pheochromocytoma; pineal parenchymal tumors of intermediate differentiation; pineoblastoma and supratentorial primitive neuroectodermal tumors; pituitary tumor; plasma cell neoplasm/multiple myeloma; pleuropulmonary blastoma; primary central nervous system lymphoma; prostate cancer; rectal cancer; renal cell cancer; renal, pelvis and/or ureter, transitional cell cancer; respiratory tract carcinoma involving the nut gene on chromosome 15; retinoblastoma; rhabdomyosarcoma; salivary gland cancer; sarcoma (e.g., Ewing family of tumors, Kaposi, soft tissue, uterine); Sezary syndrome; skin cancer (e.g., non-melanoma, melanoma, merkel cell); small cell lung cancer; small intestine cancer; soft tissue sarcoma; squamous cell carcinoma; squamous neck cancer with occult primary, metastatic; stomach cancer; supratentorial primitive neuroectodermal tumors; T-cell lymphoma, cutaneous; testicular cancer; throat cancer; thymoma and/or thymic carcinoma; thyroid cancer; transitional cell cancer of the renal, pelvis and/or ureter; trophoblastic tumor; unknown primary site carcinoma; urethral cancer; uterine cancer, endometrial; uterine sarcoma; vaginal cancer; visual pathway and/or hypothalamic glioma; vulvar cancer; Waldenström macroglobulinemia; Wilms tumor and the like. For a review, see the National Cancer Institute's Worldwide Website (cancer.gov/cancertopics/alphalist). One of skill in the art will understand that this list is exemplary only and is not exhaustive, as one of skill in the art will readily be able to identify additional cancers and/or neoplasms based on the disclosure herein.

Examples of noncancerous cellular proliferative disorders includes fibroadenoma, adenoma, intraductal papilloma, nipple adenoma, adenosis, fibrocystic disease or changes of breast, plasma cell proliferative disorder (PCPD), restenosis, atherosclerosis, rheumatoid arthritis, myofibromatosis, fibrous hamartoma, granular lymphocyte proliferative disorders, benign hyperplasia of prostate, heavy chain diseases (HCDs), lymphoproliferative disorders, psoriasis, idiopathic pulmonary fibrosis, scleroderma, cirrhosis of the liver, IgA nephropathy, mesangial proliferative glomerulonephritis, membranoproliferative glomerulonephritis, hemangiomas, vascular and non-vascular intraocular proliferative disorders and the like. One of skill in the art will understand that this list is exemplary only and is not exhaustive, as one of skill in the art will readily be able to identify additional noncancerous cellular proliferative disorders based on the disclosure herein.

In accordance with certain other examples, methods for treating viral infections are also disclosed. Treatment of viral infections is intended to include, but is not limited to, the use of a diaryloxindole compound described herein to prevent the initiation of viral protein synthesis. The term “viral infection,” as used herein, refers to one or more cells which have been infected with a virus, such as a DNA or RNA animal virus. As used herein, RNA viruses include, but are not limited to, virus families such as picornaviridae (e.g., polioviruses), reoviridae (e.g., rotaviruses), togaviridae (e.g., encephalitis viruses, yellow fever virus, rubella virus), orthomyxoviridae (e.g., influenza viruses), paramyxoviridae (e.g., respiratory syncytial virus, measles virus, mumps virus, parainfluenza virus), rhabdoviridae (e.g., rabies virus), coronaviridae, bunyaviridae, flaviviridae, filoviridae, arenaviridae, bunyaviridae, and retroviridae (e.g., human T-cell lymphotropic viruses (HTLV), human immunodeficiency viruses (HIV)). As used herein, DNA viruses include, but are not limited to, virus families such as papovaviridae (e.g., papilloma viruses), adenoviridae (e.g., adenovirus), herpesviridae (e.g., herpes simplex viruses), and poxviridae (e.g., variola viruses). In certain embodiments, the viral infection is caused by hepatitis B virus, hepatitis C virus, and/or HIV. One of skill in the art will understand that this list is exemplary only and is not exhaustive, as one of skill in the art will readily be able to identify additional viral infections based on the disclosure herein.

In accordance with other examples, methods for treating disorders associated with viral infections are disclosed. Treatment of one or more disorders associated with viral infections is intended to include, but is not limited to, the use of a diaryloxindole compound described herein to reduce or alleviate one or more symptoms of a viral infection. As used herein, the term “disorders associated with viral infection” refers to the host's response to infection by one or more viruses. Such responses include, but are not limited to neurological symptoms (e.g., encephalitis, meningoencephalitis, paralysis, myelopathy, neuropathy, aseptic meningitis, hemiparesis, dementia, dysphagia, lack of muscular coordination, impaired vision, coma, and the like), wasting symptoms (e.g., inflammatory cell infiltration, perivascular cuffing of blood vessels, demyelination, necrosis, reactive gliosis and the like), gastroenteritis symptoms (e.g., diarrhea, vomiting, cramps and the like), hepatitis symptoms (nausea, vomiting, right upper quadrant pain, raised liver enzyme levels (e.g., AST, ALT and the like), jaundice and the like), hemorrhagic fever symptoms (e.g., headache, fever, chills body pains, diarrhea, vomiting, dizziness, confusion, abnormal behavior, pharyngitis, conjunctivitis, red face, red neck, hemorrhage, organ failure and the like), oncogenic symptoms (e.g., sarcomas, leukemias and the like, as well as “rare” malignancies, e.g., Kaposi's sarcoma, oral hairy leukoplasia, lymphomas and the like), immunodeficiency symptoms (e.g., opportunistic infections, wasting, rare malignancies, neurological disease, fever, diarrhea, skin rashes and the like), lesions (e.g., warts (e.g., common wart, flat wart, deep hyperkaratotic palmoplantar wart, superficial mosaic type palmoplantar wart and the like), epidermodysplasia, mucosal lesions, ulcers and the like), and systemic symptoms (e.g., fever, chills, headache, muscle pain, bone pain, joint pain, pharyngitis, tonsillitis, sinusitis, otitis, bronchitis, pneumonia, bronchopneumonia, nausea, vomiting, increased salivation, rash, macules, lymphadenopothy, arthritis, ulcers, photosensitivity, weight loss, irritability, restlessness, anxiety, coma, death and the like). Disorders associated with viral infections are described in Fields Virology 4^{th} Ed. (2001) Lippincott, Williams & Wilkins, and the introduction to medical virology website (web.uct.ac.za/depts./mmi/jmoodie/introvi2.html), incorporated herein by reference in their entirety for all purposes. One of skill in the art will understand that this list is exemplary only and is not exhaustive, as one of skill in the art will readily be able to identify additional disorders associate with viral infections based on the disclosure herein.

In accordance with other examples, methods for treating non-proliferative, degenerative disorders associated with aberrant translation initiation using a diaryloxindole compound described herein to alleviate and/or reduce one or more symptoms associated with a non-proliferative, degenerative disorder are disclosed. Treatment of non-proliferative, degenerative diseases is intended to include, but is not limited to, the use of diaryloxindole compounds described herein. As used herein, the term “non-proliferative degenerative disorder” is intended to include, but is not limited to, diseases characterized by a loss of function of cells, tissues, and/or organs due to aberrant translation initiation. Non-proliferative degenerative disorders include, but are not limited to, disorders such as Alzheimer's disease and insulin resistance. One of skill in the art will understand that this list is exemplary only and is not exhaustive, as one of skill in the art will readily be able to identify additional non-proliferative degenerative disorders based on the disclosure herein.

In accordance with other examples, methods for treating disorders characterized by unwanted synthesis and/or abnormal accumulation of one or more mutant and/or wild-type proteins are provided. Treatment of one or more disorders associated with unwanted synthesis and/or abnormal accumulation is intended to include, but is not limited to, the use of a diaryloxindiole compound of the present invention to reduce or alleviate one or more symptoms characterized by unwanted synthesis and/or abnormal accumulation. Without intending to be bound by scientific theory, contacting a subject afflicted with a disorder characterized by unwanted synthesis and/or abnormal accumulation of one or more mutant and/or wild-type proteins with a compound described herein (e.g., a compound that can inhibit translation initiation) can reduce the load on the protein-folding machinery and, accordingly, may reduce the severity of the disorder. Disorders associated with unwanted synthesis and/or abnormal accumulation of one or more mutant and/or wild-type proteins include, but are not limited to, Tay-Sachs disease, cystic fibrosis, phenylketonuria, Fabry disease, Alzheimer's disease, Huntington's disease, Parkinson's disease, congophilic angiopathy, prion related disorders (i.e., transmissible spongiform encephalopathies such as Creutzfeldt-Jacob disease, kuru, fatal familial insomnia, scrapie, bovine spongiform encephalopathy and the like) and the like. One of skill in the art will understand that this list is exemplary only and is not exhaustive, as one of skill in the art will readily be able to identify additional disorders characterized by unwanted synthesis and/or abnormal accumulation of one or more mutant and/or wild-type proteins based on the disclosure herein.

The term “calcium releaser,” as used herein, refers to molecules which cause a sustained depletion of intracellular Ca²⁺ stores and inhibit translation initiation. Calcium releasers include, but are not limited to, molecules such as clotrimazole (CLT), fatty acids such as EPA, diaryloxindole compounds of the present invention, and the like.

In accordance with certain other examples, kits for treating one or more (1) proliferative disorders, (2) non-proliferative, degenerative disorders, (3) viral infections, and/or (4) disorders associated with viral infections are provided. In one example, the kit may comprise one or more diaryloxindole compounds of the present invention, or a combination of one or more diaryloxindole compounds of the present invention. In another example, the kit may comprise a pharmaceutically acceptable carrier. In an additional example, the kit may also include instructions for treating (1) proliferative disorders, (2) non-proliferative, degenerative disorders, (3) viral infections, and/or (4) disorders associated with viral infections. In some examples, the kit may also comprise, e.g., a buffering agent, a preservative, or a protein stabilizing agent. In other examples, the kit may also contain a control sample or a series of control samples which can be assayed and compared to the test sample contained. Other suitable components for including in the kit will be selected by the person of ordinary skill in the art, given the benefit of this disclosure.

In accordance with certain examples, diaryloxindole compounds of the present invention can be incorporated into pharmaceutical compositions suitable for administration. Such compositions typically comprise the compounds disclosed here and a pharmaceutically acceptable carrier. As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

In accordance with certain examples, a pharmaceutical composition of the invention is formulated to be compatible with its intended route of administration. Such pharmaceutical compositions may be administered by inhalation, transdermally, orally, rectally, transmucosally, intestinally, parenterally, intramuscularly, subcutaneously, intravenously or other suitable methods that will be readily selected by the person of ordinary skill in the art, given the benefit of this disclosure. For example, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

In accordance with other examples, pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMPHOR EL™ (BASF, Parsippany, N.J.), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

In accordance with other examples, sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation can be vacuum drying and freeze-drying which yields a powder of the active ingredient plus any additional desired ingredient from a previously sterile-filtered solution thereof. Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In at least certain examples, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These may be prepared according to methods known to those skilled in the art, for example, as described in U.S. Pat. No. 4,522,811, incorporated herein by reference in its entirety for all purposes.

In accordance with certain examples, pharmaceutical compositions of the invention comprise one or more diaryloxindole compounds covalently linked to a peptide (i.e., a polypeptide comprising two or more amino acids) (FIGS. 4A-4B). Peptides may be assembled sequentially from individual amino acids or by linking suitable small peptide fragments. In sequential assembly, the peptide chain is extended stepwise, starting at the C-terminus, by one amino acid per step. In fragment coupling, fragments of different lengths can be linked together, and the fragments can also be obtained by sequential assembly from amino acids or by fragment coupling of still shorter peptides.

In both sequential assembly and fragment coupling it is necessary to link the units (e.g., amino acids, peptides, compounds and the like) by forming an amide linkage, which can be accomplished via a variety of enzymatic and chemical methods. The methods described herein for formation of peptidic amide linkages are also suitable for the formation of non-peptidic amide linkages.

Chemical methods for forming the amide linkage are described in detail in standard references on peptide chemistry, including Muller, Methoden der organischen Chemie Vol. XV/2, 1-364, Thieme Verlag, Stuttgart, (1974); Stewart and Young, Solid Phase Peptide Synthesis, 31-34 and 71-82, Pierce Chemical Company, Rockford, Ill. (1984); Bodanszky et al., Peptide Synthesis, 85-128, John Wiley & Sons, New York, (1976); Practice of Peptide Synthesis, M. Bodansky, A. Bodansky, Springer-Verlag, 1994 and other standard works in peptide chemistry, incorporated herein by reference in their entirety for all purposes. Methods include the azide method, the symmetric and mixed anhydride method, the use of in situ generated or preformed active esters, the use of urethane protected N-carboxy anhydrides of amino acids and the formation of the amide linkage using coupling reagents, such as dicyclohexylcarbodiimide (DCC), diisopropylcarbodiimide (DIC), 1-ethoxycarbonyl-2-ethoxy-1,2-dihydroquinoline (EEDQ), pivaloyl chloride, 1-ethyl-3-(3-dimethylaminopropyl)carbodiimide hydrochloride (EDCI), n-propane-phosphonic anhydride (PPA), N,N-bis(2-oxo-3-oxazolidinyl)amido phosphoryl chloride (BOP-Cl), bromo-tris-pyrrolidinophosphonium hexafluorophosphate (PyBrop), diphenylphosphoryl azide (DPPA), Castro's reagent (BOP, PyBop), O-benzotriazolyl-N,N,N′,N′-tetramethyluronium salts (HBTU), O-azabenzotriazolyl-N,N,N′,N′-tetramethyluronuim salts (TATU), diethylphosphoryl cyanide (DEPCN), 2,5-diphenyl-2,3-dihydro-3-oxo-4-hydroxythiophene dioxide (Steglich's reagent; HOTDO), 1,1′-carbonyldiimidazole (CDI) and the like. The coupling reagents can be employed alone or in combination with additives such as N,N-dimethyl-4-aminopyridine (DMAP), N-hydroxy-benzotriazole (HOBt), N-hydroxybenzotriazine (HOOBt), N-hydroxysuccinimide (HOSu), 2-hydroxypyridine and the like.

In accordance with other examples, methods of modulating translation initiation for therapeutic purposes are disclosed. In one example, a method involves contacting a cell with an agent that inhibits translation initiation. An agent that inhibits translation initiation can be any one of the compounds described herein, such as a diaryloxindole compound. In at least certain examples, the compound modulates the depletion of intracellular calcium stores. Methods of modulating translation initiation can be performed in vitro (e.g., by culturing a cell with the agent) or, alternatively, in vivo (e.g., by administering the agent to a subject). Certain examples disclosed herein are directed to methods of treating an individual afflicted with a disease or disorder characterized by aberrant translation initiation. Examples of such disorders are described herein.

In one embodiment, the method involves administering an agent (e.g., an agent identified by a screening assay described herein), or combination of agents that inhibits translation initiation. As used herein, an individual afflicted with a disease or disorder is intended to include both human and non-human mammals. Examples of non-human mammals include, but are not limited to, non-human primates, horses, cows, goats, sheep, dogs, cats, mice, rats, hamsters, guinea pigs and the like.

The present invention provides for both prophylactic and therapeutic methods of treating a subject for one or more (1) proliferative disorders, (2) non-proliferative, degenerative disorders, (3) viral infections, and/or (4) disorders associated with viral infection. In one aspect, the invention provides a method for preventing in a subject, a disease or condition associated with one or more (1) proliferative disorders, (2) non-proliferative, degenerative disorders, (3) viral infections, and/or (4) disorders associated with viral infection, by administering, to the subject one or more diaryloxindole compounds described herein to modulate one or more (1) proliferative disorders, (2) non-proliferative, degenerative disorders, (3) viral infections, and/or (4) disorders associated with viral infection. Administration of a prophylactic agent can occur prior to the manifestation of symptoms, such that a disease or disorder is prevented or, alternatively, delayed in its progression.

Another aspect of the invention pertains to therapeutic methods of treating one or more (1) proliferative disorders, (2) non-proliferative, degenerative disorders, (3) viral infections, and/or (4) disorders associated with viral infection for therapeutic purposes. Accordingly, in an exemplary embodiment, a therapeutic method of the invention involves contacting a subject with a diaryloxindole compound that therapeutically treats one or more (1) proliferative disorders, (2) non-proliferative, degenerative disorders, (3) viral infections, and/or (4) disorders associated with viral infection.

One embodiment of the present invention involves a method of treating a translation initiation-associated disease or disorder which includes the step of administering a therapeutically and/or prophylactically effective amount of an agent which inhibits translation initiation to a subject. In another embodiment, a subject is administered a therapeutically and/or prophylactically effective amount that is effective to deplete intracellular calcium stores. As defined herein, a therapeutically and/or prophylactically effective amount of agent (i.e., an effective dosage) ranges from about 0.001 to 30 mg/kg body weight, from about 0.01 to 25 mg/kg body weight, from about 0.1 to 20 mg/kg body weight, from about 1 to 10 mg/kg, from about 2 to 9 mg/kg, from about 3 to 8 mg/kg, from about 4 to 7 mg/kg, or from about 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Treatment of a subject with a therapeutically and/or prophylactically effective amount of an inhibitor can include a single treatment or can include a series of treatments. It will also be appreciated that the effective dosage of in used for treatment may increase or decrease over the course of a particular treatment.

### Example I

### Synthesizing Diaryloxindole Compounds

Synthetic approaches to producing substituted diaryloxindole compounds are known. See US 2007/0099976; J. Med. Chem. 2006, 49, 3770-3773; Klumpp et al. (1998) J. Org. Chem., 63:4481 and Hewawasam et al. (2002) Bioorganic & Medicinal Chemistry Letters, 12:1023, and US2007/0299102A1 each hereby incorporated by reference in their entireties.

Specific diaryloxindole compounds of the present invention have been made as follows. Compounds described herein were purified either by re-crystallization or by column chromatography, and were characterized by ¹H nuclear magnetic resonance (NMR) and liquid-chromatography-atmospheric pressure chemical ionization-mass spectrometry (LC-APCI-MS).

### 1. Preparation of 3-(5-tert-Butyl-2-(2-(dimethylamino)ethoxy)phenyl)-3-phenylindolin-2-one (SD028)

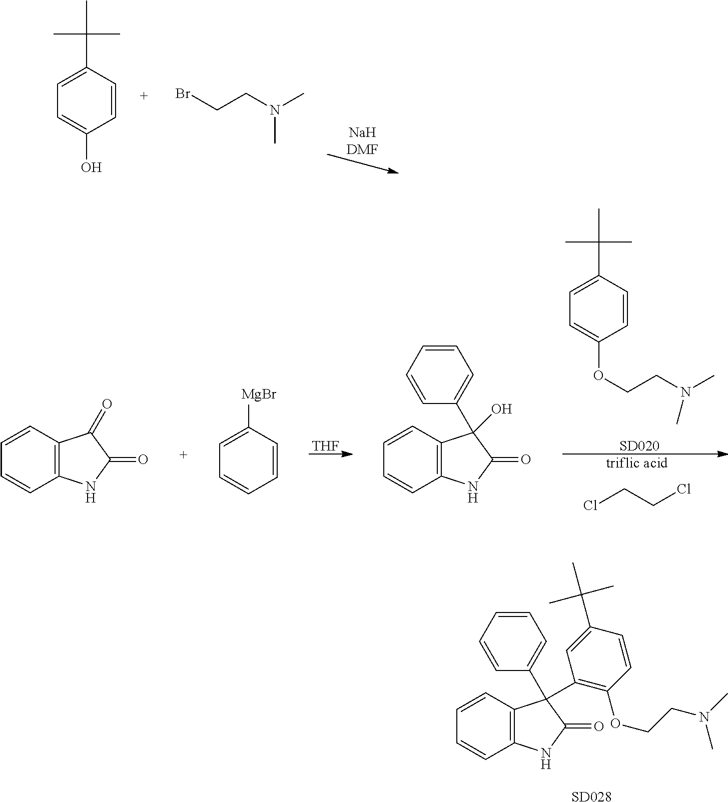

### 3-Hydroxy-3-phenylindolin-2-one (SD018)

A solution of phenylmagnesium bromide in THF (20.4 mL of a 1.0M) was added dropwise to a stirred ice-cold solution of isatin (1 g, 6.80 mmol) in anhydrous THF (20 mL) that was kept under nitrogen. The reaction was stirred at room temperature for two hours and then quenched with a saturated aqueous solution of NH₄Cl (20 mL). Water was then added (30 mL) and the aqueous phase was extracted twice with dichloromethane (2×60 mL). The organic phase was washed with brine and dried over anhydrous Na₂SO₄. The solvent was removed under vacuum and the crude was purified by crystallization from EtOAc/n-Hexane to yield 1.18 g (77%) of product SD018 as a white powder.

### 2-(4-tert-Butylphenoxy)-N,N-dimethylethanamine (SD020)

A solution of 4-tertbutylphenol (2 g, 13.33 mmol) in anhydrous DMF (25 mL) was added dropewise to a stirred mixture of NaH (0.427 g, 17.78 mmol) in anhydrous DMF (50 mL) that was kept under nitrogen. After 30 minutes, a solution of N,N-dimethylamino-ethylbromide (1.35 g, 8.89 mmol) in anhydrous DMF (25 mL) was added dropewise and the reaction mixture was left for 16 hours at 80° C. After removal of DMF under reduced pressure, the crude was dissolved in dichloromethane (200 ml) and the resulting solution was washed several times with brine and dried over anhydrous Na₂SO₄. After removal of the solvent under vacuum, the crude was purified by flash chromatography with the gradient of methanol in dichloromethane (0-10%) to yield 0.90 g (28%) of product SD020 as a colorless oil.

### 3-(5-tert-Butyl-2-(2-(dimethylamino)ethoxy)phenyl)-3-phenylindolin-2-one (SD028)

Triflic acid (790 μL, 8.90 mmol) was added to a stirred ice-cold solution of intermediates SD018 (200 mg, 0.89 mmol) and SD020 (236 mg, 1.07 mmol) in dichloromethane (5 mL) that was kept under nitrogen. After 1.5 hours, the reaction mixture was quenched over several grams of ice. The aqueous phase was then extracted twice with dichloromethane and the combined organic phases were washed with brine and dried over anhydrous Na₂SO₄. After removal of the solvent under vacuum, the crude was purified by crystallization from EtOAc/n-Hexane to yield 260 mg (68%) of product SD028 as a white powder.

1H NMR (500 MHz, DMSO, δ): 10.38 (s, 1H, NH), 7.40-7.21 (m, 7H, CH_{arom.}), 7.04-6.80 (m, 5H, CH_{arom.}), 3.85 (m, 1H, CH₂O), 3.60 (m, 1H, CH₂O), 2.30 (m, 1H, CH₂N), 2.13-2.09 (m, 7H, CH₂N, CH₃), 1.11 (s, 9H, C(CH₃)₃).

### 2. Preparation of 3-(5-tert-Butyl-2-(2-morpholinoethoxy)phenyl)-3-phenylindolin-2-one (SD029)

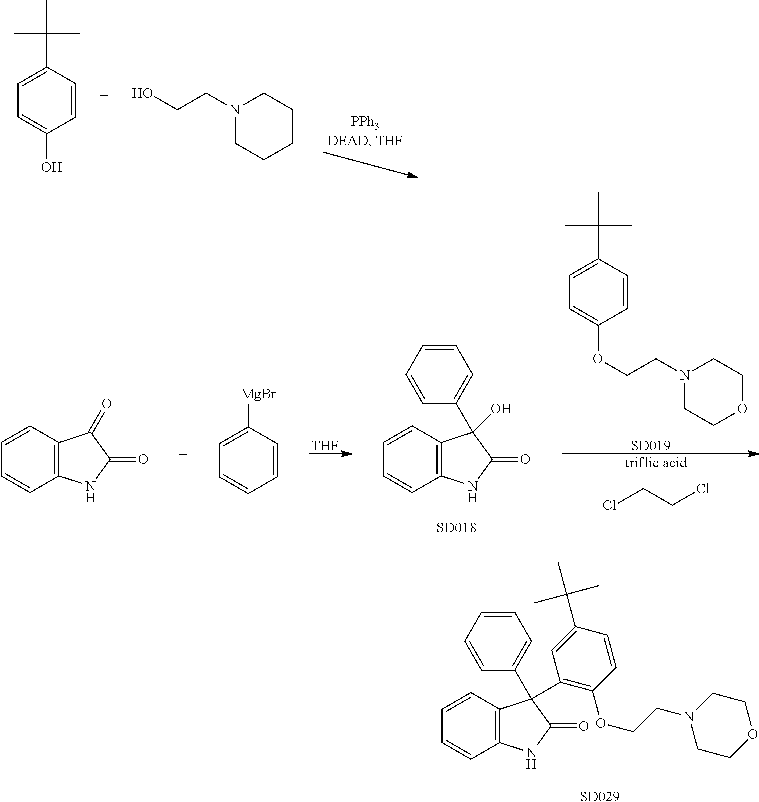

### 3-Hydroxy-3-phenylindolin-2-one (SD018)

A solution of phenylmagnesium bromide in THF (20.4 mL of a 1.0M) was added dropwise to a stirred ice-cold solution of isatin (1 g, 6.80 mmol) in anhydrous THF (20 mL) that was kept under nitrogen. The reaction was stirred at room temperature for two hours and then quenched with a saturated aqueous solution of NH₄Cl (20 mL). Water was then added (30 mL) and the aqueous phase was extracted twice with dichloromethane (2×60 mL). The organic phase was washed with brine and dried over anhydrous Na₂SO₄. The solvent was removed under vacuum and the crude was purified by crystallization from EtOAc/n-Hexane to yield 1.18 g (77%) of product SD018 as a white powder.

### 4-(2-(4-tert-Butylphenoxy)ethyl)morpholine (SD019)

A ice-cold solution of 4-tertbutylphenol (1 g, 6.67 mmol), 4(2-hydroxyethyl)morpholine (0.873 g, 6.67 mmol) and triphenylphosphine (2.27 g, 8.67 mmol) in anhydrous THF (50 mL) was stirred and kept under nitrogen for 20 minutes. This was followed by a dropewise addition of 40% DEAD in toluene (3.77 mL, 8.67 mmol) and the reaction mixture was allowed to react for 2 more hours at room temperature. After removal of the solvent under vacuum, the residue was dissolved in dichloromethane. Addition of n-Hexane formed a precipitate of triphenylphosphine oxide that was filtered off. Removal of the solvents under reduced pressure generated a crude that was purified by flash chromatography with the gradient of EtOAc in c-Hhexane (10-50%) to yield 0.96 g (55%) of product SD019 as a colorless oil.

### 3-(5-tert-Butyl-2-(2-morpholinoethoxy)phenyl)-3-phenylindolin-2-one (SD029)

Triflic acid (790 μL, 8.90 mmol) was added to a stirred ice-cold solution of intermediates SD018 (200 mg, 0.89 mmol) and SD019 (280 mg, 1.07 mmol) in dichloromethane (5 mL) that was kept under nitrogen. After 1.5 hours, the reaction mixture was quenched over several grams of ice. The aqueous phase was then extracted twice with dichloromethane and the combined organic phases were washed with brine and dried over anhydrous Na₂SO₄. After removal of the solvent under vacuum, the crude was purified by crystallization from EtOAc/n-Hexane to yield 250 mg (60%) of the product SD029 as a white powder.

1H NMR (500 MHz, DMSO, δ): 10.39 (s, 1H, NH), 7.31-7.09 (m, 8H, CH_{arom.}), 6.96-6.18 (m, 4H, CH_{arom.}), 3.90 (m, 1H, CH₂O), 3.66 (m, 1H, CH₂O), 3.50 (m, 4H, CH₂OCH₂), 2.32-2.24 (m, 6H, CH₂N, CH₂NCH₂), 1.11 (s, 9H, C(CH₃)₃).

### 3. Preparation of 3-[5-tert-butyl-2-(prop-2-yn-1-yloxy)phenyl]-3-phenylindol-2-one (SD123)

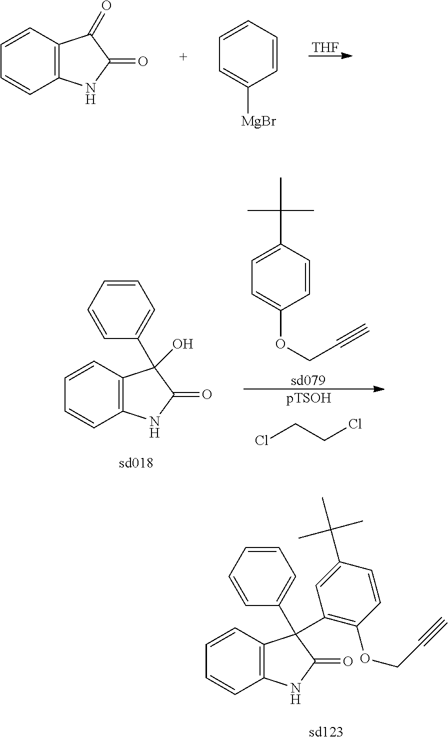

### 3-Hydroxy-3-phenylindolin-2-one (SD018)

A solution of phenylmagnesium bromide in THF (20.4 mL of a 1.0M) was added dropwise to a stirred ice-cold solution of isatin (1 g, 6.80 mmol) in anhydrous THF (20 mL) that was kept under nitrogen. The reaction was stirred at room temperature for two hours and then quenched with a saturated aqueous solution of NH₄Cl (20 mL). Water was then added (30 mL) and the aqueous phase was extracted twice with dichloromethane (2×60 mL). The organic phase was washed with brine and dried over anhydrous Na₂SO₄. The solvent was removed under vacuum and the crude was purified by crystallization from EtOAc/n-Hexane to yield 1.18 g (77%) of product SD018 as a white powder.

### 1-tert-butyl-4-(prop-2-yn-1-yloxy)benzene (SD079)

A solution of 4-tertbutylphenol (1 g, 6.67 mmol) in anhydrous DMF (25 mL) was added dropewise to a stirred mixture of NaH (0.32 g, 13.33 mmol) in anhydrous DMF (25 mL) that was kept under nitrogen. After 30 minutes, a solution of propargylbromide (0.95 g, 7.98 mmol) in anhydrous DMF (15 mL) was added dropewise and the reaction mixture was left for 16 hours at 80° C. After removal of DMF under reduced pressure, the crude was dissolved in dichloromethane (100 ml) and the resulting solution was washed several times with brine and dried over anhydrous Na₂SO₄. After removal of the solvent under vacuum, the crude was purified by flash chromatography with the gradient of EtOAc in c-Hexane (5-15%) to yield 0.72 g (57.6%) of product SD079 as a yellow oil.

### 3-[5-tert-butyl-2-(prop-2-yn-1-yloxy)phenyl]-3-phenylindol-2-one (SD123)

Triflic acid (197.5 μL, 2.22 mmol) was added to a stirred ice-cold solution of intermediates SD018 (100 mg, 0.44 mmol) and SD079 (125 mg, 0.67 mmol) in dichloromethane (5 mL) that was kept under nitrogen. After 1.5 hours, the reaction mixture was quenched over several grams of ice. The aqueous phase was then extracted twice with dichloromethane and the combined organic phases were washed with brine and dried over anhydrous Na₂SO₄. After removal of the solvent under vacuum, the crude was purified by flash chromatography with the gradient of EtOAc in c-Hexane (10-40%) to yield 98 mg (56%) of product SD123 as a white powder.

1H NMR (500 MHz, DMSO, δ): 10.40 (s, 1H, NH), 7.34-7.20 (m, 7H, CH_{arom.}), 7.01-6.83 (m, 5H, CH_{arom.}), 4.45 (d, 1H, J=16 Hz, CH₂), 4.29 (d, 1H, J=16 Hz, CH₂), 3.41 (s, 1H, CH), 1.12 (s, 9H, C(CH₃)₃).

### 4. Preparation of 3-{5-tert-butyl-2-[2-(morpholin-4-yl)ethoxy]phenyl}-3-(3,4-dimethoxy phenyl)-indol-2-one (SD152)

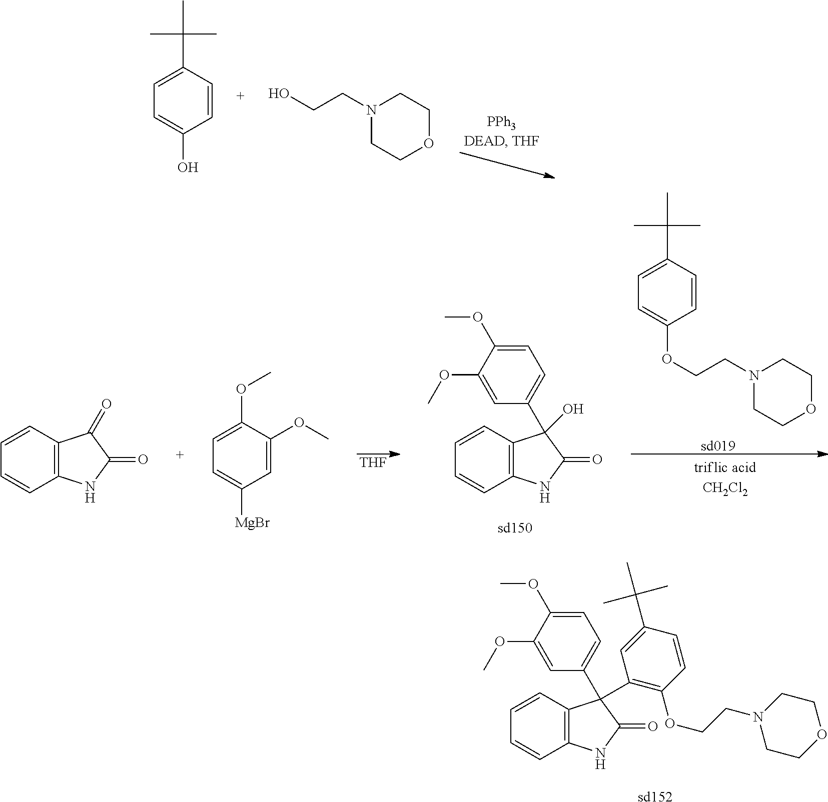

### 4-(2-(4-tert-Butylphenoxy)ethyl)morpholine (SD019)

A ice-cold solution of 4-tertbutylphenol (1 g, 6.67 mmol), 4(2-hydroxyethyl)morpholine (0.873 g, 6.67 mmol) and triphenylphosphine (2.27 g, 8.67 mmol) in anhydrous THF (50 mL) was stirred and kept under nitrogen for 20 minutes. This was followed by a dropewise addition of 40% DEAD in toluene (3.77 mL, 8.67 mmol) and the reaction mixture was allowed to react for 2 more hours at room temperature. After removal of the solvent under vacuum, the residue was dissolved in dichloromethane. Addition of n-Hexane formed a precipitate of triphenylphosphine oxide that was filtered off. Removal of the solvents under reduced pressure generated a crude that was purified by flash chromatography with the gradient of EtOAc in c-Hhexane (10-50%) to yield 0.96 g (55%) of product SD019 as a colorless oil.

### 3-(3,4-dimethoxyphenyl)-3-hydroxy-indol-2-one (Sd150)

A solution of 3,4-dimethoxyphenylmagnesium bromide in THF (20.4 mL of a 0.5 M) was added dropwise to a stirred ice-cold solution of isatin (500 mg, 3.40 mmol) in anhydrous THF (20 mL) that was kept under nitrogen. The reaction was stirred at room temperature for two hours and then quenched with a saturated aqueous solution of NH₄Cl (20 mL). Water was then added (30 mL) and the aqueous phase was extracted twice with dichloromethane (2×60 mL). The organic phase was washed with brine and dried over anhydrous Na₂SO₄. The solvent was removed under vacuum and the crude was purified by crystallization from EtOAc/n-Hexane to yield 767 mg (79%) of product SD150 as a white powder.

### 3-{5-tert-butyl-2-[2-(morpholin-4-yl)ethoxy]phenyl}-3-(3,4-dimethoxy phenyl)-indol-2-one (SD152)

Triflic acid (234 μL, 2.63 mmol) was added to a stirred ice-cold solution of intermediates SD019 (166 mg, 0.63 mmol) and SD150 (150 mg, 0.53 mmol) in dichloromethane (5 mL) that was kept under nitrogen. After 1.5 hours, the reaction mixture was quenched over several grams of ice. The aqueous phase was then extracted twice with dichloromethane and the combined organic phases were washed with brine and dried over anhydrous Na₂SO₄. After removal of the solvent under vacuum, the crude was purified by flash chromatography with the gradient of methanol in dichloromethane (2-5%) to yield 187 mg (67%) of product SD152 as a white powder.

1H NMR (500 MHz, DMSO, δ): 10.29 (s, 1H, NH), 7.21-7.19 (m, 2H, CH_{arom.}), 7.03-6.85 (m, 7H, CH_{arom.}), 6.61 (s, 1H, CH_{arom.}), 3.87 (m, 1H, CH₂O), 3.73-3.69 (m, 4H, CH₂O, OCH₃), 3.59 (s, 3H, OCH₃), 3.48 (m, 4H, CH₂OCH₂), 2.37 (m, 1H, CH₂N), 2.30-2.23 (m, 5H, CH₂N, CH₂NCH₂), 1.12 (s, 9H, C(CH₃)₃).

### 5. Preparation of 3-(4-methoxyphenyl)-3-{2-[2-(morpholin-4-yl)ethoxy]-5-(propan-2-yloxy)phenyl}-indol-2-one (SD179)

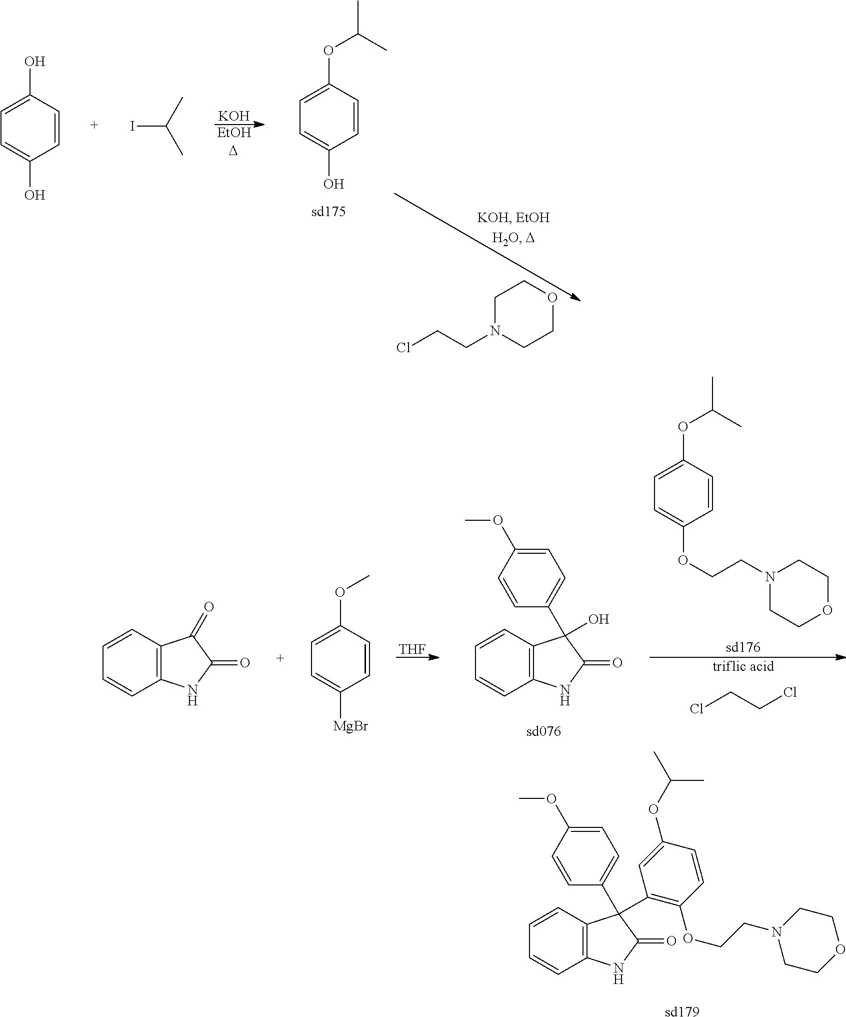

### 3-Hydroxy-3-(4-methoxyphenyl)-indol-2-one (Sd076)

A solution of 4-methoxyphenylmagnesium bromide in THF (40.8 mL of a 0.5 M) was added dropwise to a stirred ice-cold solution of isatin (1 g, 6.80 mmol) in anhydrous THF (20 mL) that was kept under nitrogen. The reaction was stirred at room temperature for two hours and then quenched with a saturated aqueous solution of NH₄Cl (20 mL). Water was then added (30 mL) and the aqueous phase was extracted twice with dichloromethane (2×80 mL). The organic phase was washed with brine and dried over anhydrous Na₂SO₄. The solvent was removed under vacuum and the crude was purified by crystallization from EtOAc/n-Hexane to yield 0.90 g (52%) of product SD076 as a white powder.

### 4-(Propan-2-yloxy)phenol (Sd175)

A solution of KOH (3.06 g, 54.50 mmol) in water (40 mL) was added to a mixture of hydroquinone (3 g, 27.24 mmol) and 2-iodopropane (4.17 g, 24.52 mmol) in ethanol (50 mL). After 16 hours at reflux, the dark brown reaction mixture was cooled down and filtered. After removal of ethanol under vacuum, the resulting aqueous phase was acidified with 2N HCl and then extracted twice with ethyl acetate (2×60 mL). The organic phase was washed with brine and dried over anhydrous Na₂SO₄. The solvent was removed under vacuum and the crude was purified by flash chromatography with the gradient of ethyl acetate in c-Hexane (0-20%) to yield 1.45 g (35%) of product SD175 as a brown oil.

### 4-{2-[4-(Propan-2-yloxy)phenoxy]ethyl}morpholine (SD176)

A solution of KOH (0.74 g, 13.16 mmol) in water (10 mL) was added to a mixture of intermediate SD175 (1 g, 6.58 mmol) and 4-(2-chloroethyl)morpholine hydrochloride (1.47 g, 24.52 mmol) in ethanol (20 mL). After 16 hours at reflux, the reaction mixture was cooled down, ethanol was removed under vacuum and the resulting aqueous phase was extracted twice with ethyl acetate (2×30 mL). The organic phase was washed with brine and dried over anhydrous Na₂SO₄. The solvent was removed under vacuum and the crude was purified by flash chromatography with the gradient of ethyl acetate in c-Hexane (20-80%) to yield 0.94 g (54%) of product SD176 as a yellow oil.

### 3-(4-Methoxyphenyl)-3-{2-[2-(morpholin-4-yl)ethoxy]-5-(propan-2-yloxy)phenyl}-indol-2-one (SD179)

Triflic acid (261 μL, 2.94 mmol) was added to a stirred ice-cold solution of intermediates SD076 (150 mg, 0.59 mmol) and SD176 (188 mg, 0.71 mmol) in dichloromethane (5 mL) that was kept under nitrogen. After 1.5 hours, the reaction mixture was quenched over several grams of ice. The aqueous phase was then extracted twice with dichloromethane and the combined organic phases were washed with brine and dried over anhydrous Na₂SO₄. After removal of the solvent under vacuum, the crude was purified by reverse phase flash chromatography with the gradient of acetonitrile in water (20-60%) to yield 108 mg (36%) of product SD179 as a white powder.

1H NMR (500 MHz, MeOD, δ): 7.27-7.24 (m, 2H, CH_{arom.}), 7.00-6.80 (m, 8H, CH_{arom.}), 6.53 (m, 1H, CH_{arom.}), 4.42 (m, 1H, CH(CH₃)₂), 4.16 (m, 2H, CH₂O), 3.87 (m, 4H, CH₂OCH₂), 3.78 (s, 3H, OCH₃), 3.42 (m, 2H, CH₂N), 3.24 (m, 4H, CH₂NCH₂), 1.19 (d, 3H, J=6 Hz, CHCH₃), 0.60 (d, 3H, J=6 Hz, CHCH₃).

### 6. Preparation of methyl 3-{5-tert-butyl-2-[2-(morpholin-4-yl)ethoxy]phenyl}-3-(4-methoxyphenyl)-2-oxoindole-7-carboxylate (SD203)

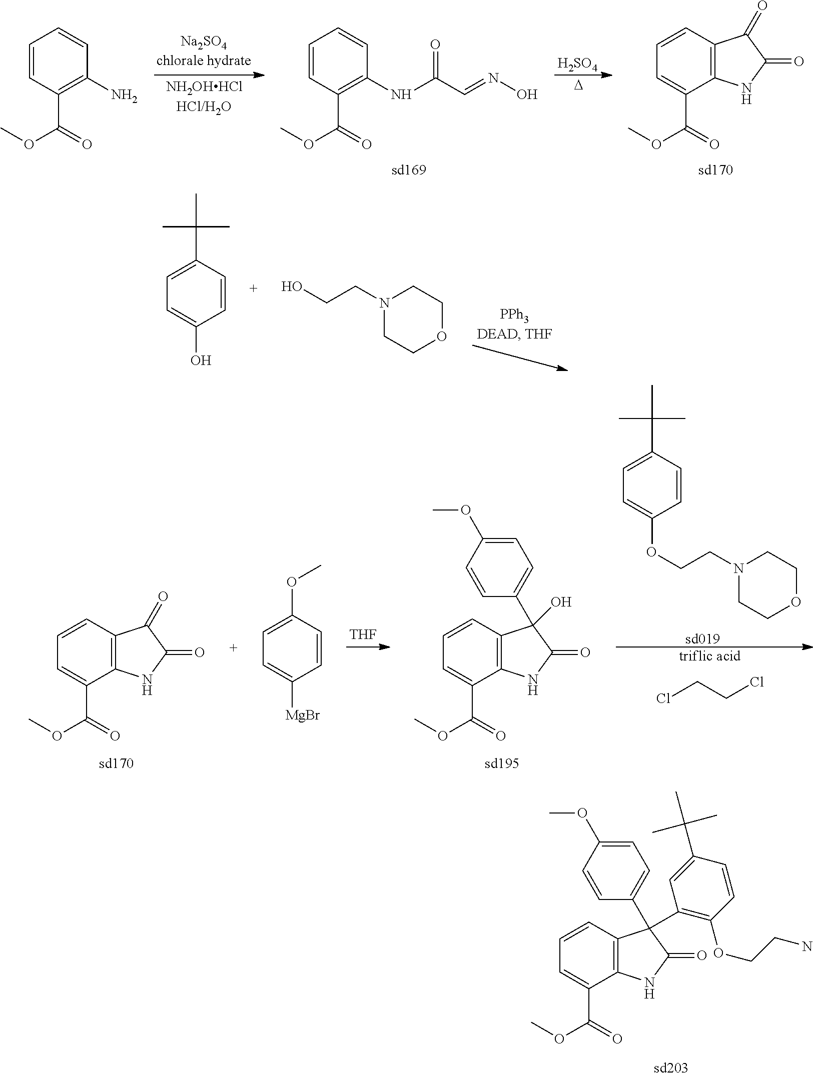

### Methyl 2-[2-(methylimino)acetamido]benzoate (SD169)

Hydroxylamine hydrochloride (13.7 g, 198 mmol), chloral hydrate (12 g, 73 mmol), methylanthranilate (10 g, 64 mmol) in water (110 mL) and finally 12M HCl (7.2 g, 198 mmol) were added in sequence to a well stirred suspension of sodium sulfate (72.3 g, 509 mmol) in water (160 mL) at 60° C. The mixture temperature was risen to 100° C. After two hours, the solution was cooled to room temperature and filtered. The isolated solid was washed with water and then dichloromethane and dried under reduce pressure overnight to yield 10.5 g of intermediate SD170 (72.4%) as a white powder which was taken to the next step without further purification.

### Methyl 2,3-dioxoindole-7-carboxylate (SD170)

Intermediate SD169 (10 g, 43.8 mmol) was added in small portions over 10 minutes to well stirred sulphuric acid (18M, 50 mL). After complete addition, the temperature was risen to 80° C. and stirred at this temperature for 20 min. The brown reaction mixture was then cooled down and slowly poured into ice (100 g) and water (100 mL). The yellow-orange precipitate was filtered, washed with water several times and finally dissolved in a saturated solution of NaHCO₃ (300 mL). The aqueous phase was extracted three times with dichloromethane (3×400 mL). The organic phase was washed with water and brine and was dried over anhydrous Na₂SO₄. The solvent was removed under vacuum to yield 3.5 g of product SD170 (39%) as a yellow powder which was taken to the next step without any further purification.

### 4-(2-(4-tert-Butylphenoxy)ethyl)morpholine (SD019)

A ice-cold solution of 4-tertbutylphenol (1 g, 6.67 mmol), 4(2-hydroxyethyl)morpholine (0.873 g, 6.67 mmol) and triphenylphosphine (2.27 g, 8.67 mmol) in anhydrous THF (50 mL) was stirred and kept under nitrogen for 20 minutes. This was followed by a dropewise addition of 40% DEAD in toluene (3.77 mL, 8.67 mmol) and the reaction mixture was allowed to react for 2 more hours at room temperature. After removal of the solvent under vacuum, the residue was dissolved in dichloromethane. Addition of n-Hexane formed a precipitate of triphenylphosphine oxide that was filtered off. Removal of the solvents under reduced pressure generated a crude that was purified by flash chromatography with the gradient of EtOAc in c-Hhexane (10-50%) to yield 0.96 g (55%) of product SD019 as a colorless oil.

### Methyl 3-hydroxy-3-(4-methoxyphenyl)-2-oxoindole-7-carboxylate (Sd195)

A solution of 4-methoxyphenylmagnesium bromide in THF (14.6 mL of a 0.5M) was added dropwise to a stirred ice-cold solution compound SD170 (1 g, 4.88 mmol) in anhydrous THF (20 mL) that was kept under nitrogen. The reaction was stirred at room temperature for two hours and then quenched with a saturated aqueous solution of NH₄Cl (10 mL). Water was then added (30 mL) and the aqueous phase was extracted twice with dichloromethane (2×80 mL). The organic phase was washed with brine and dried over anhydrous Na₂SO₄. The solvent was removed under vacuum and the crude was purified by crystallization from EtOAc/n-Hexane to yield 1.060 mg (69%) of product SD195 as a white powder.

### Methyl 3-{5-tert-butyl-2-[2-(morpholin-4-yl)ethoxy]phenyl}-3-(4-methoxy phenyl)-2-oxoindole-7-carboxylate (SD203)

Triflic acid (852 μL, 9.5 mmol) was added to a stirred ice-cold solution of intermediates SD019 (302 mg, 1.15 mmol) and SD195 (300 mg, 0.96 mmol) in dichloromethane (10 mL) that was kept under nitrogen. After 1.5 hours, the reaction mixture was quenched over several grams of ice. The aqueous phase was then extracted twice with dichloromethane and the combined organic phases were washed with brine and dried over anhydrous Na₂SO₄. After removal of the solvent under vacuum, the crude was purified by reverse phase flash chromatography with the gradient of methanol in water (50-100%) to yield 180 mg (34%) of product SD203 as a white powder.

### 7. Preparation of 33-{5-tert-butyl-2-[2-(piperazin-1-yl)ethoxy]phenyl}-3-phenylindol-2-one (SD049)

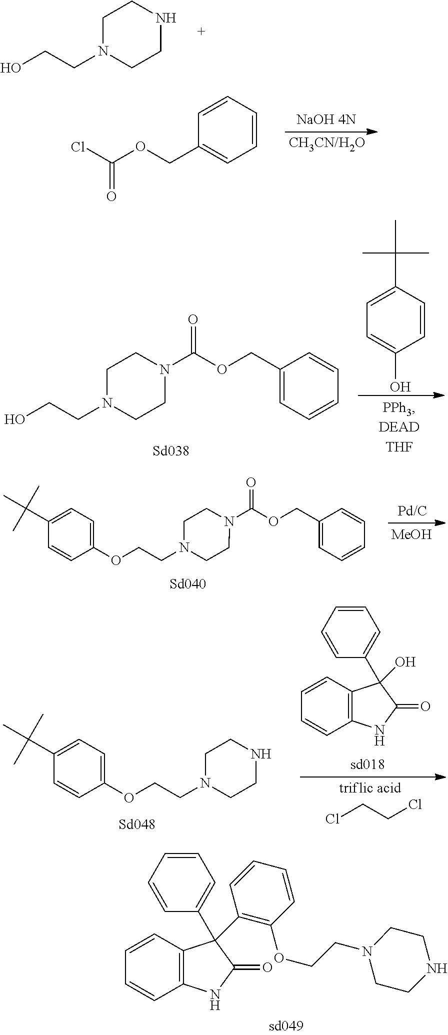

### 3-Hydroxy-3-phenylindolin-2-one (SD018)

A solution of phenylmagnesium bromide in THF (20.4 mL of a 1.0M) was added dropwise to a stirred ice-cold solution of isatin (1 g, 6.80 mmol) in anhydrous THF (20 mL) that was kept under nitrogen. The reaction was stirred at room temperature for two hours and then quenched with a saturated aqueous solution of NH₄Cl (20 mL). Water was then added (30 mL) and the aqueous phase was extracted twice with dichloromethane (2×60 mL). The organic phase was washed with brine and dried over anhydrous Na₂SO₄. The solvent was removed under vacuum and the crude was purified by crystallization from EtOAc/n-Hexane to yield 1.18 g (77%) of product SD018 as a white powder.

### Benzyl 4-(2-hydroxyethyl)piperazine-1-carboxylate (SD038)

Benzylchloroformate (4.7 g, 27.6 mmol) in acetonitrile (30 mL) was added dropewise over 30 min to a solution of 1-(2-hydroxyethyl)piperazine (3 g, 23 mmol) in water (30 mL) via an isobar cylindrical funnel. The pH was maintained around 8-9 by addition of a solution of NaOH 4N. The reaction was stirred overnight at room temperature. The mixture was first extracted with dichloromethane (100 mL) in order to remove the diprotected compound and then acidified with HCl 4N. The acidic aqueous phase was extracted twice with dichloromethane (2×100 mL). The organic phase was washed with brine and dried over anhydrous Na₂SO₄. The solvent was removed under vacuum and the crude was purified by flash chromatography with the gradient of methanol in dichloromethane (0-8%) to yield 5.41 g (90%) of product SD038 as a colorless oil.

### Benzyl 4-[2-(4-tert-butylphenoxy)ethyl]piperazine-1-carboxylate (SD040)

A ice-cold solution of 4-tertbutylphenol (0.85 g, 5.67 mmol), intermediate SD038 (1.5 g, 5.67 mmol) and triphenylphosphine (1.93 g, 7.37 mmol) in anhydrous THF (50 mL) was stirred and kept under nitrogen for 20 minutes. This was followed by a dropewise addition of 40% DEAD in toluene (3.2 mL, 7.37 mmol) and the reaction mixture was allowed to react for 2 more hours at room temperature. After removal of the solvent under vacuum, the residue was dissolved in dichloromethane. Addition of n-Hexane formed a precipitate of triphenylphosphine oxide that was filtered off. Removal of the solvents under reduced pressure generated a crude that was purified by flash chromatography with the gradient of EtOAc in c-Hhexane (40-100%) to yield 1.62 g (72%) of product SD040 as a colorless oil.

### 1-[2-(4-tert-butylphenoxy)ethyl]piperazine (SD048)

Palladium on charcoal 10% (75 mg, 60 mg per mmol) was carefully added to a solution of intermediate SD040 (500 mg, 1.26 mmol) in methanol (10 mL). A catalytic amount of acetic acid was added. The reaction mixture was flushed under hydrogen at room temperature and atmospheric pressure for 1.5 hours and then filtered over celite. The solvent was then removed under vacuum to yield 320 mg (97%) of product SD048 as a yellow oil which was taken to the next step without further purification.

### 33-{5-tert-butyl-2-[2-(piperazin-1-yl)ethoxy]phenyl}-3-phenylindol-2-one (SD049)

Triflic acid (690 μL, 7.76 mmol) was added to a stirred ice-cold solution of intermediates SD018 (175 mg, 0.78 mmol) and SD048 (300 mg, 0.93 mmol) in dichloromethane (5 mL) that was kept under nitrogen. After 1.5 hours, the reaction mixture was quenched over several grams of ice. The aqueous phase was then extracted twice with dichloromethane and the combined organic phases were washed with brine and dried over anhydrous Na₂SO₄. After removal of the solvent under vacuum, the crude was purified by crystallization from EtOAc/n-Hexane to yield 146 mg (40%) of product SD049 as a white powder.

1H NMR (500 MHz, DMSO, δ): 10.40 (s, 1H, NH), 7.30-7.12 (m, 8H, CH_{arom.}), 6.95-6.820 (m, 4H, CH_{arom.}), 3.87 (m, 1H, CH₂O), 3.60 (m, 1H, CH₂O), 2.60 (m, 4H, CH₂O CH₂), 2.28-2.08 (m, 6H, CH₂N, CH₂N CH₂), 1.11 (s, 9H, C(CH₃)₃).

### 8. Preparation of 3-{5-tert-butyl-2-[2-(morpholin-4-yl)ethoxy]phenyl}-3-(4-methoxy phenyl)-indol-2-one (SD139)

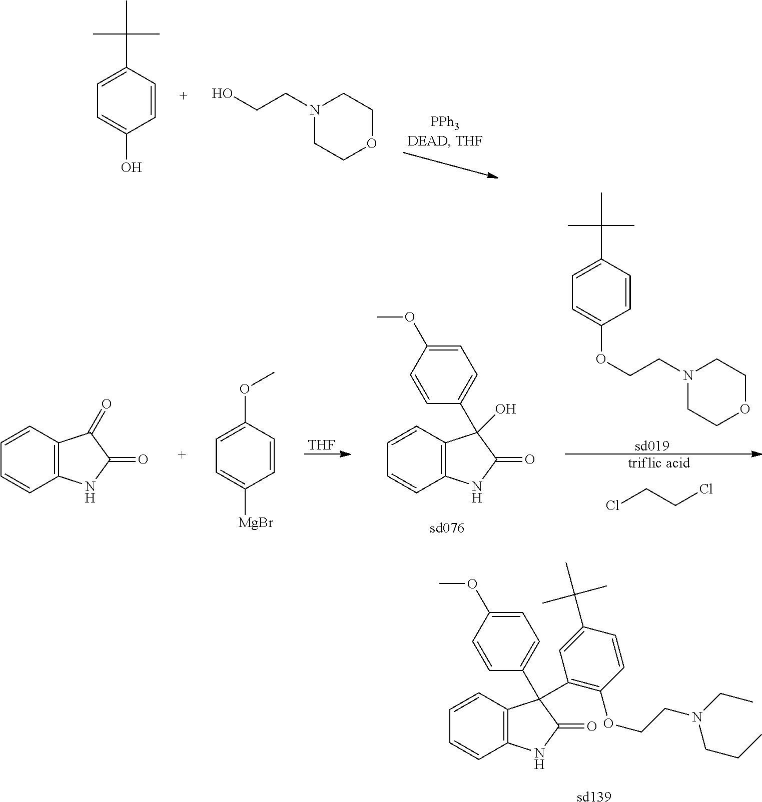

### 4-(2-(4-tert-Butylphenoxy)ethyl)morpholine (SD019)

A ice-cold solution of 4-tertbutylphenol (1 g, 6.67 mmol), 4(2-hydroxyethyl)morpholine (0.873 g, 6.67 mmol) and triphenylphosphine (2.27 g, 8.67 mmol) in anhydrous THF (50 mL) was stirred and kept under nitrogen for 20 minutes. This was followed by a dropewise addition of 40% DEAD in toluene (3.77 mL, 8.67 mmol) and the reaction mixture was allowed to react for 2 more hours at room temperature. After removal of the solvent under vacuum, the residue was dissolved in dichloromethane. Addition of n-Hexane formed a precipitate of triphenylphosphine oxide that was filtered off. Removal of the solvents under reduced pressure generated a crude that was purified by flash chromatography with the gradient of EtOAc in c-Hhexane (10-50%) to yield 0.96 g (55%) of product SD019 as a colorless oil.

### 3-hydroxy-3-(4-methoxyphenyl)-indol-2-one (Sd076)

A solution of 4-methoxyphenylmagnesium bromide in THF (40.8 mL of a 0.5 M) was added dropwise to a stirred ice-cold solution of isatin (1 g, 6.80 mmol) in anhydrous THF (20 mL) that was kept under nitrogen. The reaction was stirred at room temperature for two hours and then quenched with a saturated aqueous solution of NH₄Cl (20 mL). Water was then added (30 mL) and the aqueous phase was extracted twice with dichloromethane (2×80 mL). The organic phase was washed with brine and dried over anhydrous Na₂SO₄. The solvent was removed under vacuum and the crude was purified by crystallization from EtOAc/n-Hexane to yield 0.90 g (52%) of product SD076 as a white powder.

### 3-{5-tert-butyl-2-[2-(morpholin-4-yl)ethoxy]phenyl}-3-(4-methoxyphenyl)-indol-2-one (SD139)

Triflic acid (350 μL, 3.92 mmol) was added to a stirred ice-cold solution of intermediates SD019 (247.5 mg, 0.94 mmol) and SD076 (200 mg, 0.78 mmol) in dichloromethane (5 mL) that was kept under nitrogen. After 1.5 hours, the reaction mixture was quenched over several grams of ice. The aqueous phase was then extracted twice with dichloromethane and the combined organic phases were washed with brine and dried over anhydrous Na₂SO₄. After removal of the solvent under vacuum, the crude was purified by flash chromatography with the gradient of methanol in ethyl acetate (2-8%) to yield 165 mg (42%) of product SD139 as a white powder.

1H NMR (500 MHz, DMSO, δ): 10.32 (s, 1H, NH), 7.21-7.19 (m, 4H, CH_{arom.}), 7.00-6.83 (m, 7H, CH_{arom.}), 3.87 (m, 1H, CH₂O), 3.73-3.67 (m, 4H, CH₂O, OCH₃), 3.49 (m, 4H, CH₂OCH₂), 2.36 (m, 1H, CH₂N), 2.26 (m, 5H, CH₂N, CH₂NCH₂), 1.12 (s, 9H, C(CH₃)₃).

### 9. Preparation of 3-{5-tert-butyl-2-[2-(morpholin-4-yl)ethoxy]phenyl}-3-[4-(dimethyl amino)phenyl]-indol-2-one (SD154)

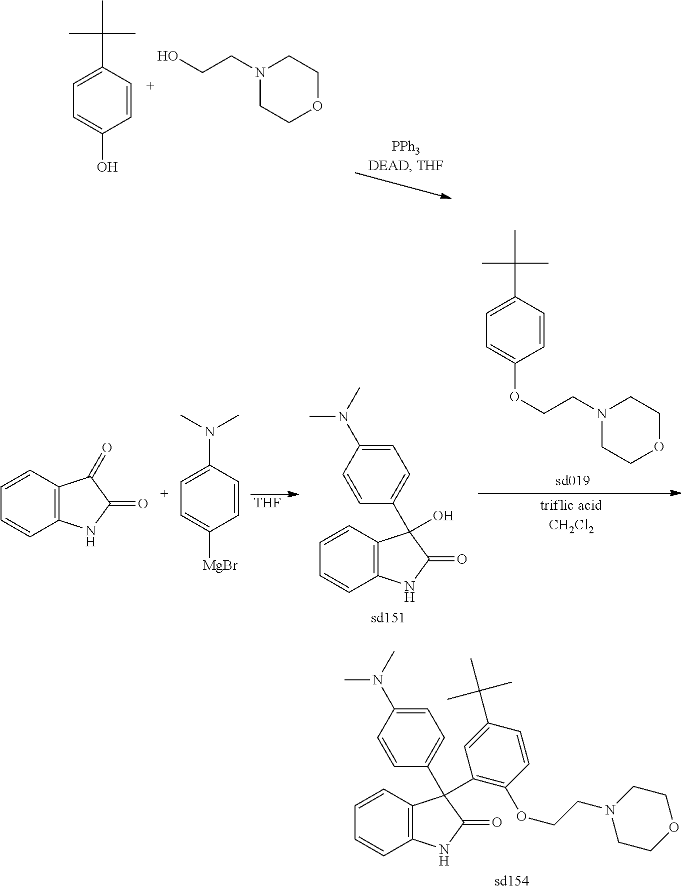

### 4-(2-(4-tert-Butylphenoxy)ethyl)morpholine (SD019)

A ice-cold solution of 4-tertbutylphenol (1 g, 6.67 mmol), 4(2-hydroxyethyl)morpholine (0.873 g, 6.67 mmol) and triphenylphosphine (2.27 g, 8.67 mmol) in anhydrous THF (50 mL) was stirred and kept under nitrogen for 20 minutes. This was followed by a dropewise addition of 40% DEAD in toluene (3.77 mL, 8.67 mmol) and the reaction mixture was allowed to react for 2 more hours at room temperature. After removal of the solvent under vacuum, the residue was dissolved in dichloromethane. Addition of n-Hexane formed a precipitate of triphenylphosphine oxide that was filtered off. Removal of the solvents under reduced pressure generated a crude that was purified by flash chromatography with the gradient of EtOAc in c-Hhexane (10-50%) to yield 0.96 g (55%) of product SD019 as a colorless oil.

### 3-[4-(dimethylamino)phenyl]-3-hydroxy-indol-2-one (Sd151)

A solution of 4-(dimethylamino)phenylmagnesium bromide in THF (20.4 mL of a 0.5 M) was added dropwise to a stirred ice-cold solution of isatin (500 mg, 3.40 mmol) in anhydrous THF (20 mL) that was kept under nitrogen. The reaction was stirred at room temperature for two hours and then quenched with a saturated aqueous solution of NH₄Cl (20 mL). Water was then added (30 mL) and the aqueous phase was extracted twice with dichloromethane (2×60 mL). The organic phase was washed with brine and dried over anhydrous Na₂SO₄. The solvent was removed under vacuum and the crude was purified by crystallization from EtOAc/n-Hexane to yield 834 mg (91%) of product SD151 as a white powder.

### 3-{5-tert-butyl-2-[2-(morpholin-4-yl)ethoxy]phenyl}-3-[4-(dimethyl amino)phenyl]-indol-2-one (SD154)

Triflic acid (422 μL, 2.80 mmol) was added to a stirred ice-cold solution of intermediates SD019 (177 mg, 0.54 mmol) and SD151 (120 mg, 0.45 mmol) in dichloromethane (5 mL) that was kept under nitrogen. After 1.5 hours, the reaction mixture was quenched over several grams of ice. The aqueous phase was then extracted twice with dichloromethane and the combined organic phases were washed with brine and dried over anhydrous Na₂SO₄. After removal of the solvent under vacuum, the crude was purified by flash chromatography with the gradient of methanol in dichloromethane (2-5%) to yield 176 mg (77%) of product SD154 as a white powder.

1H NMR (500 MHz, DMSO, δ): 10.21 (s, 1H, NH), 7.201-7.16 (m, 3H, CH_{arom.}), 7.08 (m, 1H, CH_{arom.}), 6.90-6.84 (m, 5H, CH_{arom.}), 6.66 (m, 2H, CH_{arom.}), 3.85 (m, 1H, CH₂O), 3.69 (m, 1H, CH₂O), 3.48 (t, J=4.5 Hz, 4H, CH₂O, CH₂OCH₂), 2.86 (s, 6H, CH₃N), 2.36 (m, 1H, CH₂N), 2.28-2.21 (m, 5H, CH₂N, CH₂NCH₂), 1.12 (s, 9H, C(CH₃)₃).

### 10. Preparation of 3-{5-tert-butyl-2-[2-(morpholin-4-yl)ethoxy]phenyl}-5-methoxy-3-(4-methoxyphenyl)-indol-2-one (SD185)

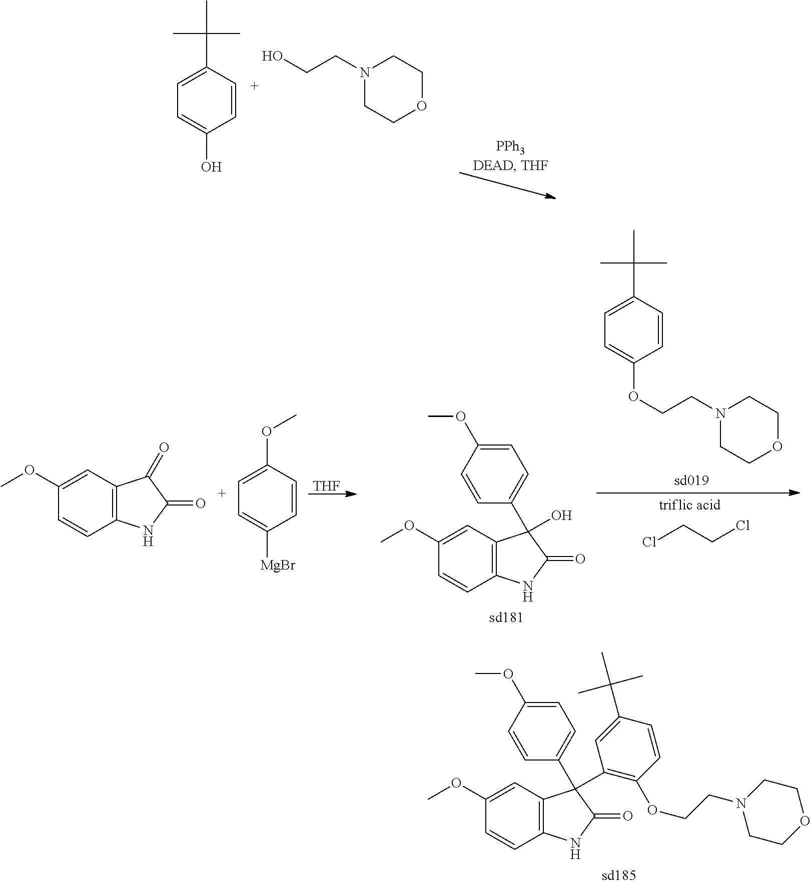

### 4-(2-(4-tert-Butylphenoxy)ethyl)morpholine (SD019)

A ice-cold solution of 4-tertbutylphenol (1 g, 6.67 mmol), 4(2-hydroxyethyl)morpholine (0.873 g, 6.67 mmol) and triphenylphosphine (2.27 g, 8.67 mmol) in anhydrous THF (50 mL) was stirred and kept under nitrogen for 20 minutes. This was followed by a dropewise addition of 40% DEAD in toluene (3.77 mL, 8.67 mmol) and the reaction mixture was allowed to react for 2 more hours at room temperature. After removal of the solvent under vacuum, the residue was dissolved in dichloromethane. Addition of n-Hexane formed a precipitate of triphenylphosphine oxide that was filtered off. Removal of the solvents under reduced pressure generated a crude that was purified by flash chromatography with the gradient of EtOAc in c-Hhexane (10-50%) to yield 0.96 g (55%) of product SD019 as a colorless oil.

### 3-Hydroxy-5-methoxy-3-(4-methoxyphenyl)-indol-2-one (Sd181)

A solution of 4-methoxyphenylmagnesium bromide in THF (17.0 mL of a 0.5M) was added dropwise to a stirred ice-cold solution of 5-methoxyisatin (500 mg, 2.82 mmol) in anhydrous THF (20 mL) that was kept under nitrogen. The reaction was stirred at room temperature for two hours and then quenched with a saturated aqueous solution of NH₄Cl (10 mL). Water was then added (20 mL) and the aqueous phase was extracted twice with dichloromethane (2×50 mL). The organic phase was washed with brine and dried over anhydrous Na₂SO₄. The solvent was removed under vacuum and the crude was purified by crystallization from EtOAc/n-Hexane to yield 690 mg (86%) of product SD181 as a white powder.

### 3-{5-tert-butyl-2-[2-(morpholin-4-yl)ethoxy]phenyl}-5-methoxy-3-(4-methoxyphenyl)-indol-2-one (SD185)

Triflic acid (233 μL, 2.63 mmol) was added to a stirred ice-cold solution of intermediates SD019 (166 mg, 0.63 mmol) and SD181 (150 mg, 0.53 mmol) in dichloromethane (5 mL) that was kept under nitrogen. After 1.5 hours, the reaction mixture was quenched over several grams of ice. The aqueous phase was then extracted twice with dichloromethane and the combined organic phases were washed with brine and dried over anhydrous Na₂SO₄. After removal of the solvent under vacuum, the crude was purified by flash chromatography with the gradient of methanol in ethyl acetate (0-10%) to yield 108 mg (39%) of product SD185 as a white powder.

1H NMR (500 MHz, DMSO, δ): 10.17 (s, 1H, NH), 7.23-7.16 (m, 3H, CH_{arom.}), 6.90-6.79 (m, 6H, CH_{arom.}), 6.60 (s, 1H, CH_{arom.}), 3.89 (m, 1H, CH₂O), 3.72-3.69 (m, 4H, CH₂O, OCH₃), 3.65 (s, 3H, OCH₃), 3.49 (m, 4H, CH₂OCH₂), 2.34 (m, 1H, CH₂N), 2.27-2.21 (m, 5H, CH₂N, CH₂NCH₂), 1.12 (s, 9H, C(CH₃)₃).

### 11. Preparation of 3-{5-tert-butyl-2-[2-(morpholin-4-yl)ethoxy]phenyl}-5-iodo-3-(4-methoxyphenyl)indol-2-one (SD204)

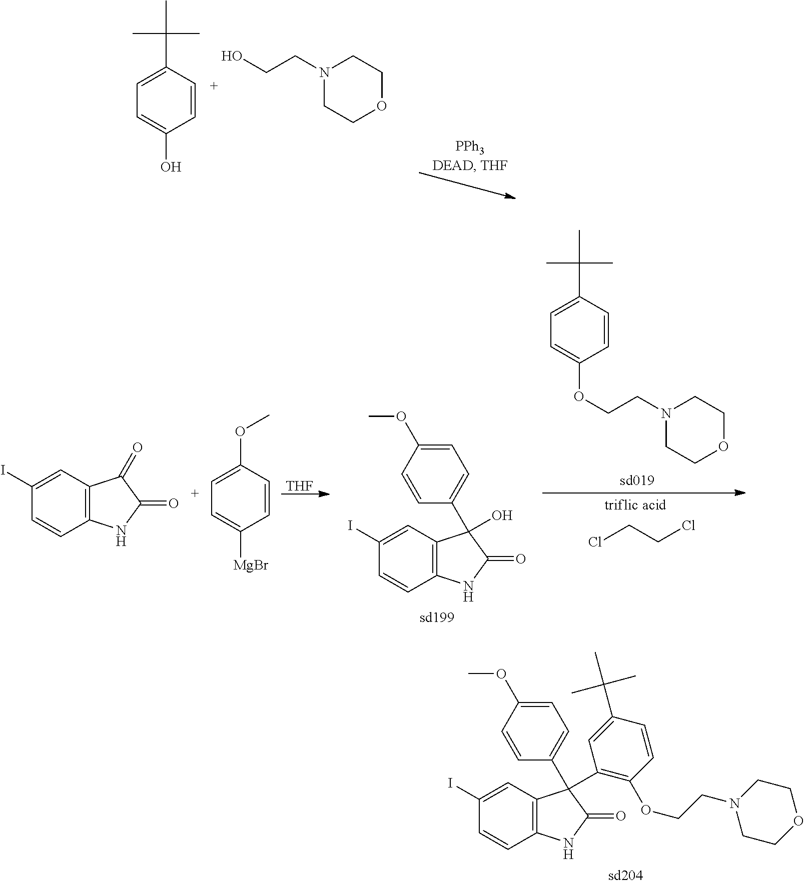

### 4-(2-(4-tert-Butylphenoxy)ethyl)morpholine (SD019)

A ice-cold solution of 4-tertbutylphenol (1 g, 6.67 mmol), 4(2-hydroxyethyl)morpholine (0.873 g, 6.67 mmol) and triphenylphosphine (2.27 g, 8.67 mmol) in anhydrous THF (50 mL) was stirred and kept under nitrogen for 20 minutes. This was followed by a dropewise addition of 40% DEAD in toluene (3.77 mL, 8.67 mmol) and the reaction mixture was allowed to react for 2 more hours at room temperature. After removal of the solvent under vacuum, the residue was dissolved in dichloromethane. Addition of n-Hexane formed a precipitate of triphenylphosphine oxide that was filtered off. Removal of the solvents under reduced pressure generated a crude that was purified by flash chromatography with the gradient of EtOAc in c-Hhexane (10-50%) to yield 0.96 g (55%) of product SD019 as a colorless oil.

### 3-Hydroxy-5-iodo-3-(4-methoxyphenyl)indol-2-one (Sd199)

A solution of 4-methoxyphenylmagnesium bromide in THF (11 mL of a 0.5M) was added dropwise to a stirred ice-cold solution of 5-iodoisatin (500 mg, 1.83 mmol) in anhydrous THF (20 mL) that was kept under nitrogen. The reaction was stirred at room temperature for two hours and then quenched with a saturated aqueous solution of NH₄Cl (10 mL). Water was then added (20 mL) and the aqueous phase was extracted twice with dichloromethane (2×50 mL). The organic phase was washed with brine and dried over anhydrous Na₂SO₄. The solvent was removed under vacuum and the crude was purified by crystallization from EtOAc/n-Hexane to yield 533 mg (76%) of product SD199 as a white powder.

### 3-{5-tert-butyl-2-[2-(morpholin-4-yl)ethoxy]phenyl}-5-iodo-3-(4-methoxyphenyl)indol-2-one (SD204)

Triflic acid (175 μL, 1.97 mmol) was added to a stirred ice-cold solution of intermediates SD019 (124 mg, 0.47 mmol) and SD199 (150 mg, 0.394 mmol) in dichloromethane (5 mL) that was kept under nitrogen. After 1.5 hours, the reaction mixture was quenched over several grams of ice. The aqueous phase was then extracted twice with dichloromethane and the combined organic phases were washed with brine and dried over anhydrous Na₂SO₄. After removal of the solvent under vacuum, the crude was purified by crystallization from EtOAc/n-Hexane to yield 122 mg (49%) of product SD204 as a white powder.

1H NMR (500 MHz, DMSO, δ): 10.48 (s, 1H, NH), 7.56 (d, J=7.5 Hz, 1H, CH_{arom.}), 7.25-7.15 (m, 4H, CH_{arom.}), 6.93-6.90 (m, 3H, CH_{arom.}), 6.80 (s, 1H, CH_{arom.}), 6.75 (d, J=7.5 Hz, 1H, CH_{arom.}), 3.87 (m, 1H, CH₂O), 3.77-3.63 (m, 4H, CH₂O, OCH₃), 3.50 (m, 4H, CH₂OCH₂), 2.31-2.23 (m, 6H, CH₂N, CH₂NCH₂), 1.12 (s, 9H, C(CH₃)₃).

### 12. Preparation of HY016, HY003, HY017

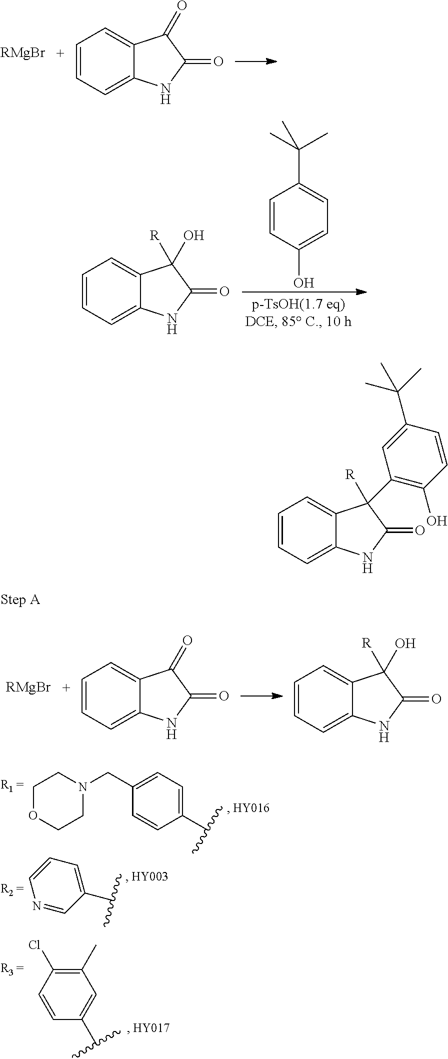

A solution of arylmagnesium bromide in THF (7.5 mmol, 2 equivalents) was added dropwise to a stirred ice-cold solution of isatin (3.75 mmol, 1 equivalent) in anhydrous THF (20 mL) that was kept under nitrogen. After stirring the reaction mixture for 2 h at 0° C. followed by 6 h at room temperature it was quenched with a saturated aqueous solution of NH₄Cl (100 mL). The aqueous solution was extracted with CH₂Cl₂ (100 mL) and the separated organic layer was washed with water followed by brine and dried over anhydrous Na₂SO₄. The solvent was removed under vacuum and the crude was purified by flash silica gel chromatography.

### 3-Hydroxy-3-(Pyridin-3-yl)Indolin-2-One

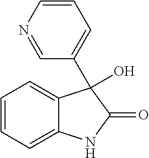 [SMILES: O=C1Nc2ccccc2C1(O)c1cccnc1]

The crude was purified by flash column chromatography on silica gel with the gradient of EtOAc in cyclohexane (30-100%) to afford 140 mg (yield 17%) of the anticipated product as a yellow solid. ESI-MS (MW calcd. 226.23) m/z=227.03 (M+H)⁺.

### 3-(4-(Morpholinomethyl)phenyl)Indolin-2-One

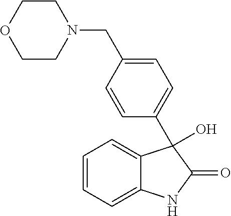 [SMILES: O=C1Nc2ccccc2C1(O)c1ccc(CN2CCOCC2)cc1]

The crude was purified by flash column chromatography on silica gel with the gradient of EtOAc in cyclohexane (10-60%) to afford 800 mg (yield 66%) of the anticipated product as a yellow oil. ESI-MS (MW calcd. 324.37) m/z=325.10 (M+H)⁺.

### 3-(4-Chloro-3-Methylphenyl)Indolin-2-One

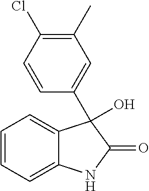 [SMILES: Cc1cc(C2(O)C(=O)Nc3ccccc32)ccc1Cl]

The crude was purified by flash column chromatography on silica gel with the gradient of methanol in dichlormethane (5-25%) to afford 542 mg (yield 53%) of the anticipated product as a yellow solid. ESI-MS (MW calcd. 273.71) m/z=256.02 (M⁺-H₂O).

### Step B

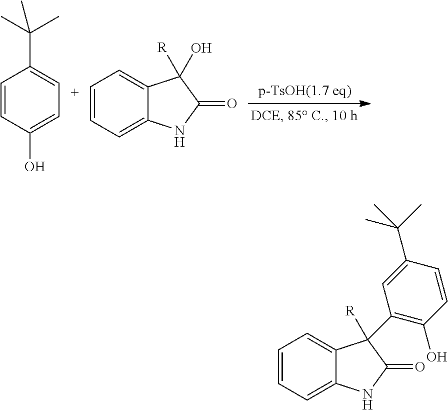

p-TsOH (1 mmol, 190 mg) was added to a solution of 4-tert-butylphenol (135 mg, 0.9 mmol) and 3-hydroxy-3-arylindolin-2-one (0.62 mmol) in dry 1,2-dichloroethane. The stirred reaction mixture was heated for 10 h at 85° C. The residue obtained after the removal of the solvent under vacuum was purified by Flash Chromatography on silica gel.

### 3-(5-tert-Butyl-2-Hydroxyphenyl)-3-(Pyridin-3-yl)Indolin-2-One (HY003)

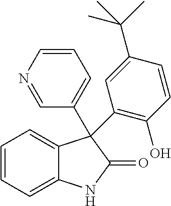 [SMILES: CC(C)(C)c1ccc(O)c(C2(c3cccnc3)C(=O)Nc3ccccc32)c1]

The crude was purified by flash column chromatography on silica gel with the gradient of methanol in dichlormethane (0-20%) to afford 30 mg (yield 18%, purity is 97.2%) of white solid. ¹H NMR (CD₃OD, 400 MHz) in ppm: δ 1.20 (s, 9H), 6.72 (d, J=8 Hz, 1H), 7.02-7.10 (m, 3H), 7.20-7.23 (m, 1H), 7.29-7.33 (m, 1H), 7.41 (d, J=8 Hz, 1H), 7.62-7.65 (m, 1H), 8.00-8.03 (m, 1H), 8.46 (s, 1H), 8.58 (s, 1H); ¹³C NMR (CD₃OD, 100 MHz) in ppm: δ 30.9, 33.8, 60.0, 110.4, 116.1, 122.7, 126.0, 126.1, 126.2, 126.4, 129.0, 131.6, 140.4, 141.8, 142.2, 144.5, 153.0, 179.9. RP-HPLC on a C18 Xbridge column retention time 5.96 min, employing a linear gradient system of acetonitrile-water: 30%-70% B in A for 10 min followed by 70%-100% B in A for 5 min, where A is 0.1% AcOH in H₂O and B is 0.1% AcOH in acetonitrile; ESI-MS (MW calcd. 358.43) m/z=359.16 (M+H)⁺.

### 3-(5-tert-butyl-2-hydroxyphenyl)-3-(4-(Morpholinomethyl)phenyl)Indolin-2-One (HY016)

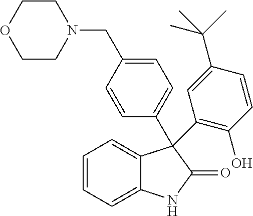 [SMILES: CC(C)(C)c1ccc(O)c(C2(c3ccc(CN4CCOCC4)cc3)C(=O)Nc3ccccc32)c1]

The crude was purified by flash column chromatography on silica gel with the gradient of EtOAc in cyclohexane (0-20%) to afford 430 mg (yield 92%, purity is 97%) of white solid. ¹H NMR (DMSO, 400 MHz) in ppm: δ 1.06 (s, 9H), 2.27 (s, 4H), 3.33 (s, 4H), 3.55 (s, 2H), 6.58 (d, J=8.0 Hz, 1H), 6.67 (s, 1H), 6.86 (d, J=8.0 Hz, 1H), 6.91-7.02 (m, 2H), 7.09 (d, J=8.0 Hz, 2H), 7.16 (t, 1H), 7.28 (s, 3H), 7.46 (d, J=8.0 Hz, 2H); RP-HPLC on a C18 Xbridge column, retention time 7.18 min, employing a linear gradient system of acetonitrile-water: 10%-60% B in A for 10 min followed by 60%-100% B in A for 3 min, where A is 0.1% AcOH in H₂O and B is 0.1% AcOH in acetonitrile; ESI-MS (MW calcd. 456.58) m/z=457.30 (M+H)⁺.

### 3-(5-tert-Butyl-2-Hydroxyphenyl)-3-(4-Chloro-3-Methylphenyl)Indolin-2-One (HY017)

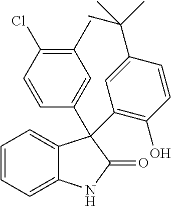 [SMILES: Cc1cc(C2(c3cc(C(C)(C)C)ccc3O)C(=O)Nc3ccccc32)ccc1Cl]

The crude was purified by flash column chromatography on silica gel with the gradient of EtOAc in cyclohexane (5-25%) to afford 280 mg (yield 74%, purity is 98.1%) of white solid. ¹H NMR (CD₃OD, 500 MHz) in ppm: δ 1.14 (s, 9H), 2.23 (s, 3H), 6.69 (d, J=10.5 Hz, 1H), 6.95-6.99 (m, 3H), 7.04-7.07 (m, 2H), 7.13-7.16 (m, 1H), 7.20-7.23 (m, 3H); ¹³C NMR (CD₃OD, 125 MHz) in ppm: δ 19.2, 30.9, 33.8, 61.2, 110.1, 116.1, 122.1, 125.7, 126.0, 126.8, 127.1, 127.2, 128.2, 128.5, 130.8, 133.1, 133.3, 135.6, 139.2, 141.8, 141.9, 153.4, 182.3; RP-HPLC on a C18 Xbridge column, retention time 9.35 min, employing a linear gradient system of acetonitrile-water: 50%-100% B in A for 15 min. Where A is 0.1% AcOH in water and B is 0.1% AcOH in acetonitrile; ESI-MS (MW calcd. 405.92) m/z=406.17 (M+H)⁺.

### 13. Preparation of HY032, HY040, HY0443

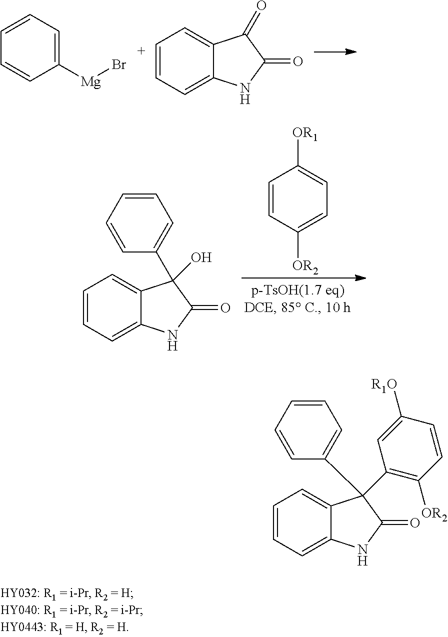

### Step A Syntheses of 3-Hydroxy-3-Phenylindolin-2-One

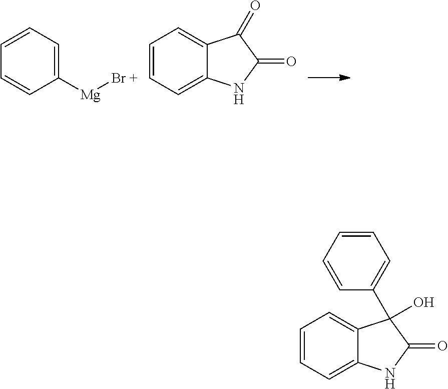

A solution of phenylmagnesium bromide in THF (120 mmol, 1.6 equivalents) was added dropwise to a stirred ice-cold solution of isatin (75 mmol, 120 mL of 1M PhMgBr in THF, 1.0 equivalent) in anhydrous THF (100 mL) that was kept under nitrogen. After stirring the reaction mixture for 2 h at 0° C. followed by 6 h at room temperature it was quenched with a saturated aqueous solution of NH₄Cl (100 mL). The aqueous solution was extracted with CH₂Cl₂ (100 mL) and the separated organic layer was washed with water followed by brine and dried over anhydrous Na₂SO₄. The solvent was removed under vacuum. The crude product was recrystallized with EtOAc twice to afford 11 g (yield 65%) of the anticipated product as a white solid. ESI-MS (MW calcd. 225.04) m/z=207.8 (M⁺-H₂O).

### Synthesis of 4-Isopropoxyphenol

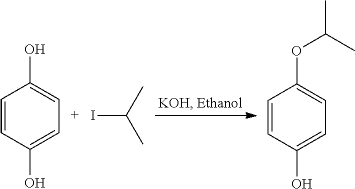

To a solution of hydroquinone (5.57 g, 50 mmol, 1.0 equivalent) and 2-iodopropane (3.37 mL, 33 mmol, 0.66 equivalent) in ethanol was added a solution of potassium hydroxide (7.85 g, 50 mmol, 1.0 equivalent) in water (10 mL). The dark brown solution was then refluxed for 16 hours. The solution was concentrated and the remaining aqueous phase was acidified with 2N HCl and extracted with ethyl acetate (3×200 mL). The combined organic layers were washed with brine, dried over magnesium sulfate and filtered. And the organic solution was concentrated and purified by Flash Chromatography on silica gel (5˜35% ethyl acetate in cyclohexane) to afford 1.9 g (yield 38%). ESI-MS (MW calcd. 152.19) m/z=150.90 (M−H)⁻.

### Synthesis of 1,4-Diisopropoxybenzene

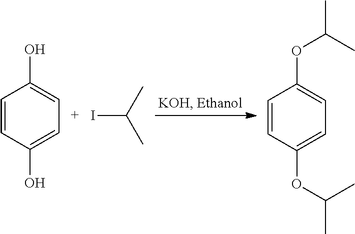

To a solution of hydroquinone (2.8 g, 25 mmol, 1.0 equivalent) and 2-iodopropane (6.74 mL, 66 mmol, 2.6 equivalents) in ethanol was added a solution of potassium hydroxide (3.3 g, 60 mmol, 2.4 equivalents) in water (10 mL). The dark brown solution was then refluxed for 16 hours. The solution was concentrated and the remaining aqueous phase was acidified with 2N HCl and extracted with ethyl acetate (3×200 mL). The combined organic layers were washed with brine, dried over magnesium sulfate and filtered. And the organic solution was concentrated and purified by flash chromatography on silica gel (0˜20% ethyl acetate in cyclohexane) to afford 2.8 g (yield 58%). ESI-MS (MW calcd. 194.27) m/z=193.94 (M⁺).

### Step B

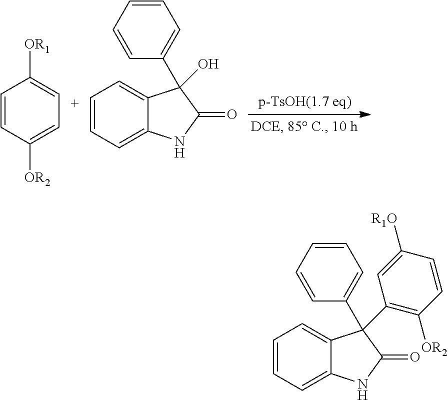

p-TsOH (1.7 mmol, 323 mg, 1.7 equivalents) was added to a solution of substituted phenol or 1,4-diisopropoxybenzene (1.2 mmol, 1.2 equivalents) and 3-hydroxy-3-phenylindolin-2-one (1 mmol, 225 mg, 1.0 equivalent) in dry 1,2-dichloroethane. The stirred reaction mixture was heated for 10 h at 85° C. The residue obtained after the removal of the solvent under vacuum to be purified.

### 3-(2-Hydroxy-5-Isopropoxyphenyl)-3-Phenylindolin-2-One (HY032)

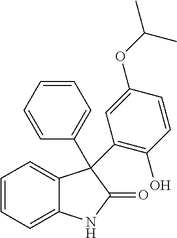 [SMILES: CC(C)Oc1ccc(O)c(C2(c3ccccc3)C(=O)Nc3ccccc32)c1]

The crude was purified by Prepared HPLC with the gradient of acetonitrile in water (30%-75% for 25 min, retention time 14.73 min) to afford 290 mg (yield 81%, purity is 99.8%) of white solid. ¹H NMR (DMSO, 400 MHz) in ppm: δ 1.11 (t, 6H), 4.20 (m, 1H), 6.25 (d, J=2.8 Hz, 1H), 6.62 (d, J=8.8 Hz, 1H), 6.68-6.71 (m, 1H), 6.90-6.97 (m, 2H), 7.07 (d, J=7.2 Hz, 1H), 7.19 (t, 1H), 7.31-7.34 (m, 5H), 9.07 (s, 1H), 10.43 (s, 1H); ¹³C NMR (DMSO, 100 MHz) in ppm: δ 22.4, 22.5, 60.5, 70.4, 110.0, 115.6, 116.7, 119.3, 121.8, 126.3, 127.9, 128.5, 128.7, 129.1, 130.6, 133.2, 140.0, 143.1, 149.7, 150.2, 180.0; RP-HPLC on a C18 Xbridge column, retention time 8.62 min, employing a linear gradient system of acetonitrile-water: 30%-100% B in A for 15 min. where A is 0.1% AcOH in water and B is 0.1% AcOH in acetonitrile; ESI-MS (MW calcd. 359.42) m/z=360.04 (M+H)⁺.

### 3-(2,5-Diisopropoxyphenyl)-3-Phenylindolin-2-One (HY040)

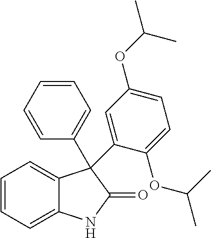 [SMILES: CC(C)Oc1ccc(OC(C)C)c(C2(c3ccccc3)C(=O)Nc3ccccc32)c1]

The crude was purified by flash column chromatography on silica gel with the gradient of EtOAc in cyclohexane (0-25%) to afford 280 mg (yield 74%, purity is 98.5%) of white solid. ¹H NMR (DMSO, 400 MHz) in ppm: δ 0.48 (d, J=6 Hz, 3H), 1.06-1.13 (m, 9H), 4.20-4.26 (m, 1H), 4.20-4.26 (m, 1H), 6.19 (d, J=2.4 Hz, 1H), 6.72-6.78 (m, 2H), 6.86-6.95 (m, 3H), 7.17-7.21 (m, 1H), 7.29-7.38 (m, 5H) 10.34 (s, 1H); ¹³C NMR (DMSO, 100 MHz) in ppm: δ 20.1, 21.8, 22.5, 60.1, 69.3, 70.2, 109.9, 113.7, 114.1, 119.5, 121.5, 126.1, 120.1, 128.4, 129.4, 133.3, 139.2, 149.3, 150.6, 179.8. RP-HPLC on a C18 Xbridge column, retention time 11.32 min, employing a linear gradient system of acetonitrile-water: 30%-100% B in A for 15 min. Where A is 0.1% AcOH in water and B is 0.1% AcOH in acetonitrile; ESI-MS (MW calcd. 401.5) m/z=402.0 (M+H)⁺.

### 3-(2,5-Dihydroxyphenyl)-3-Phenylindolin-2-One (HY0443)

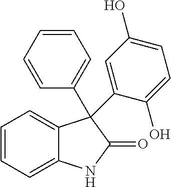 [SMILES: O=C1Nc2ccccc2C1(c1ccccc1)c1cc(O)ccc1O]

The crude was purified by flash column chromatography on silica gel with the gradient of EtOAc in cyclohexane (10-45%) to afford 280 mg (yield 88%, purity is 94.7%) of white solid. RP-HPLC on a C18 Xbridge column, retention time 11.43 min, employing a linear gradient system of acetonitrile-water: 0-45% B in A for 10 min followed by 45% to 100% B in A for 5 min. Where A is 0.1% AcOH in water and B is 0.1% AcOH in acetonitrile; ESI-MS (MW calcd. 317.34) m/z=318.11 (M+H)⁺.

### 14. Synthesis of 3-(2-Hydroxy-5-Galactopyranosylphenyl)-3-Phenylindolin-2-One (HY043)

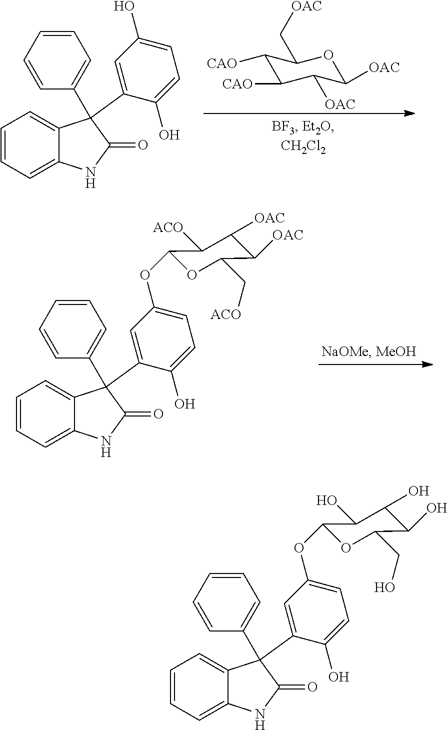

β-D-Galactose pentaacetate (936 mg, 2.4 mmol, 1.2 equivalents) and 3-(2,5-dihydroxyphenyl)-3-phenylindolin-2-one (634 mg, 2 mmol, 1 equivalent) were dissolved in dry dichloromethane (5 mL) in the presence of molecular sieves (4 Å, 3 g). Boron trifluoride-diethyl ether (0.32 mL, 2.4 mmol, 1.2 equivalents) was added dropwise. The reaction was stirred at room temperature for 24 h under nitrogen atmosphere. The reaction mixture was filtered and to the filtrate was added dichloromethane (100 mL). The organic layer was washed with sat. aq. NaHCO₃ (3×40 mL), 0.5 N NaOH (3×33 mL) and water, dried (MgSO₄), filtered and concentrated in vacuo, affording the crude product as a brown oil. The crude product was not purified and used into the next step. The crude product which was obtained from previous step was dissolved in sodium methoxide in methanol (0.5 N, 10 mL). The reaction mixture was stirred at room temperature. After 3.5 h the reaction mixture was made slightly acidic by addition of acetic acid to the mixture, and then all solvent was removed in vacuo. The product was purified by flash chromatography on the reverse phase column with the gradient of acetonitrile in water (0%-40%) to afford 147 mg (yield 15%, purity is 99.3%) of white solid. ¹H NMR (DMSO, 400 MHz) in ppm: δ 3.29-3.49 (m, 5H), 3.65 (d, J=2.8 Hz, 1H), 4.48 (t, 1H), 4.82 (t, 4H), 6.38 (t, 1H), 6.57 (d, J=8.4 Hz, 1H), 6.77-7.18 (m, 5H), 7.31 (s, 5H), 9.15 (s, 1H), 10.4 (d, 1H); ¹³C NMR (CD₃OD, 100 MHz) in ppm: δ 60.4, 68.5, 70.9, 73.9, 75.7, 102.2, 102.5, 110.0, 116.2, 116.4, 121.8, 126.2, 127.9, 128.5, 128.7, 129.0, 130.5, 133.1, 133.3, 139.8, 143.1, 150.3, 150.6, 180.0; RP-HPLC on a C18 Xbridge column retention time 9.66 min, employing a linear gradient system of acetonitrile-water: 0%-45% B in A for 10 min followed by 45%-100% B in A for 5 min, where A is 0.1% AcOH in H₂O and B is 0.1% AcOH in acetonitrile; ESI-MS (MW calcd. 479.48) m/z=478.15 (M−H)⁻.

### 15. Preparation of HY021, HY033, HY037, HY048, HY050, HY051

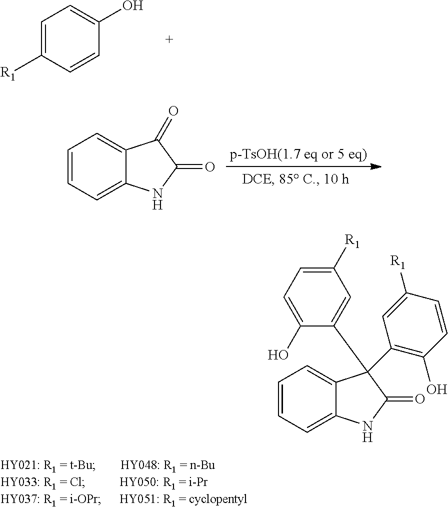

p-TsOH (1.9 g, 10 mmol, 5 equivalents; or 0.64 g, 3.4 mmol, 1.7 equivalents) was added to a solution of substituted phenol (4.4 mmol, 2.2 equivalents) and isatin (294 mg, 2 mmol, 1.0 equivalent) in dry 1,2-dichloroethane. The stirred reaction mixture was heated for 10 h at 85° C. The residue obtained after the removal of the solvent under vacuum was purified by Flash Chromatography.

### 3,3-Bis(5-tert-Butyl-2-Hydroxyphenyl)Indolin-2-One (HY021)

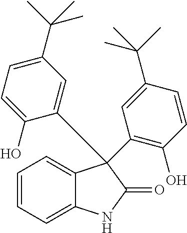 [SMILES: CC(C)(C)c1ccc(O)c(C2(c3cc(C(C)(C)C)ccc3O)C(=O)Nc3ccccc32)c1]

1.7 equivalents of p-TsOH was using the reaction. The crude was purified by flash column chromatography on silica gel with the gradient of EtOAc in cyclohexane (5-25%) to afford 576 mg (yield 67%, purity is 99.5%) of white solid. ¹H NMR (CD₃OD, 400 MHz) in ppm: δ 1.12 (s, 18H), 1.45 (s, 3H), 6.61 (m, 1H), 6.79-6.85 (m, 3H), 6.99-7.10 (m, 4H), 7.35-7.29 (m, 2H); ¹³C NMR (CD₃OD, 100 MHz) in ppm: δ 26.8, 30.7, 61.1, 105.0, 110.1, 114.7, 121.8, 125.0, 126.1, 126.2, 126.3, 126.4, 128.1, 132.5, 142.1, 185.1; RP-HPLC on a C18 Xbridge column, retention time 10.40 min, employing a linear gradient system of acetonitrile-water: 50%-100% B in A for 15 min. Where A is 0.1% AcOH in water and B is 0.1% AcOH in acetonitrile; ESI-MS (MW calcd. 429.55) m/z=430.30 (M+H)⁺.

### 3,3-Bis(5-Chloro-2-Hydroxyphenyl)Indolin-2-One (HY033)

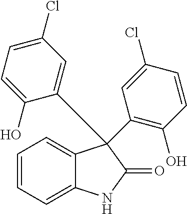 [SMILES: O=C1Nc2ccccc2C1(c1cc(Cl)ccc1O)c1cc(Cl)ccc1O]

1.7 equivalents of p-TsOH was using the reaction. The crude was purified by Prepared HPLC with the gradient of acetonitrile in water (20%-75% in 30 min, retention time 22.52 min) to afford 140 mg (yield 18%, purity is 98.73%) of white solid. ¹H NMR (DMSO, 400 MHz) in ppm: δ 6.72 (s, 3H), 6.91 (s, 1H), 6.95-7.06 (m, 3H), 7.17 (s, 1H), 7.24-7.28 (m, 2H), 10.1 (s, 2H), 11.2 (s, 1H); ¹³C NMR (DMSO, 100 MHz) in ppm: δ 60.4, 111.0, 122.9, 126.6, 129.3, 131.6, 142.6, 182.4; RP-HPLC on a C18 Xbridge column, retention time 9.67 min, employing a linear gradient system of acetonitrile-water: 30%-100% B in A for 15 min. Where A is 0.1% AcOH in water and B is 0.1% AcOH in acetonitrile; ESI-MS (MW calcd. 386.23) m/z=386.03 (M⁺).

### 3,3-Bis(2-Hydroxy-5-Isopropoxyphenyl)Indolin-2-One (HY037)

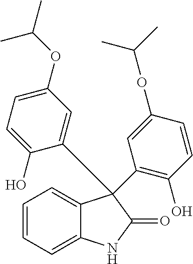 [SMILES: CC(C)Oc1ccc(O)c(C2(c3cc(OC(C)C)ccc3O)C(=O)Nc3ccccc32)c1]

1.7 equivalents of p-TsOH was using the reaction. The crude was purified by flash column chromatography on silica gel with the gradient of EtOAc in cyclohexane (5-45%) to afford 86 mg (yield 10%, purity is 98.03%) of white solid. ¹H NMR (DMSO, 400 MHz) in ppm: δ 1.08-1.14 (m, 12H), 14.22-4.28 (m, 1H), 4.31-4.37 (m, 1H), 6.25 (q, 2H), 6.53-6.59 (m, 1H), 6.68-6.82 (m, 4H), 6.94-7.01 (m, 2H), 7.25 (t, 1H), 8.76 (s, 1H), 9.58 (s, 1H), 10.92 (s, 1H); RP-HPLC on a C18 Xbridge column, retention time 8.14 min, employing a linear gradient system of acetonitrile-water: 30%-100% B in A for 15 min. Where A is 0.1% AcOH in water and B is 0.1% AcOH in acetonitrile; ESI-MS (MW calcd. 433.5) m/z=434.1 (M+H)⁺.

### 3,3-Bis(5-Butyl-2-Hydroxyphenyl)Indolin-2-One (HY048)

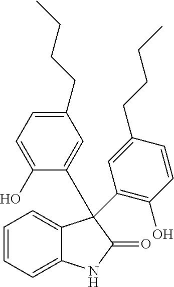 [SMILES: CCCCc1ccc(O)c(C2(c3cc(CCCC)ccc3O)C(=O)Nc3ccccc32)c1]

5.0 equivalents of p-TsOH was using the reaction. The product was purified by flash chromatography on the reverse phase column with the gradient of acetonitrile in water (50%-100%) to afford 710 mg (yield 82.8%, purity is 99.97%) of white solid. ¹H NMR (CDCl₃, 400 MHz) in ppm: δ 0.88 (t, 6H), 1.29 (m, 4H), 1.46 (m, 4H), 2.46 (t, 4H), 6.30 (d, J=7.6 Hz, 1H), 6.46 (t, 1H), 6.61-6.64 (m, 2H), 6.72-6.76 (m, 2H), 6.81 (d, J=7.6 Hz, 1H), 6.95 (dd, J=8.0 Hz and J=18.8 Hz, 2H), 7.15 (d, J=8.0 Hz, 1H), 9.14 (s, 1H); ¹³C NMR (CDCl₃, 100 MHz) in ppm: δ 14.2, 22.3, 33.8, 35.0, 60.7, 111.4, 116.8, 119.7, 122.9, 123.2, 125.8, 126.4, 128.6, 129.1, 129.3, 129.7, 130.2, 131.6, 134.7, 135.5, 140.7, 150.7, 155.2, 185.6; RP-HPLC on a C18 Xbridge column, retention time 7.27 min, employing a linear gradient system of acetonitrile-water: 70%-100% B in A for 15 min. Where A is 0.1% AcOH in water and B is 0.1% AcOH in acetonitrile; ESI-MS (MW calcd. 429.5) m/z=430.2 (M+H)⁺.

### 3,3-Bis(2-Hydroxy-5-Isopropylphenyl)Indolin-2-One (HY050)

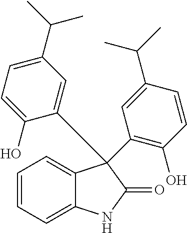 [SMILES: CC(C)c1ccc(O)c(C2(c3cc(C(C)C)ccc3O)C(=O)Nc3ccccc32)c1]

5.0 equivalents of p-TsOH was using the reaction. The crude was purified by flash column chromatography on silica gel with the gradient of EtOAc in cyclohexane (5-35%) to afford 633 mg (yield 79%, purity is 99.56%) of white solid. ¹H NMR (CDCl₃, 400 MHz) in ppm: δ 1.13 (q, 12H), 2.74 (m, 2H), 6.25 (d, J=7.2 Hz, 1H), 6.42 (t, 1H), 7.64-6.83 (m, 5H), 7.00 (d, J=8.4 Hz, 2H), 7.19-7.21 (m, 1H), 9.14 (s, 1H); ¹³C NMR (CDCl₃, 100 MHz) in ppm: δ 14.4, 21.3, 24.2, 24.5, 33.4, 60.7, 111.4, 116.7, 119.6, 122.8, 123.1, 125.8, 126.5, 126.7, 127.7, 127.9, 128.0, 128.6, 131.6, 140.7, 140.8, 141.5, 150.8, 155.2, 185.5; RP-HPLC on a C18 Xbridge column, retention time 5.78 min, employing a linear gradient system of acetonitrile-water: 70%-100% B in A for 15 min. Where A is 0.1% AcOH in water and B is 0.1% AcOH in acetonitrile; ESI-MS (MW calcd. 401.5) m/z=402.1 (M+H)⁺.

### 3,3-Bis(5-Cyclopentyl-2-Hydroxyphenyl)Indolin-2-One (HY051)

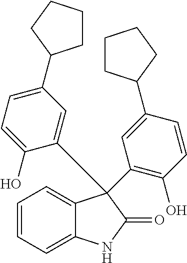 [SMILES: O=C1Nc2ccccc2C1(c1cc(C2CCCC2)ccc1O)c1cc(C2CCCC2)ccc1O]

5.0 equivalents of p-TsOH was using the reaction. The crude was purified by flash column chromatography on silica gel with the gradient of EtOAc in cyclohexane (5-35%) to afford 688 mg (yield 76%, purity is 98.74%) of white solid. ¹H NMR (CDCl₃, 400 MHz) in ppm: δ 1.44 (s, 4H), 1.57-1.69 (m, 8H), 1.95 (s, 4H), 2.81-2.86 (m, 2H), 6.28 (d, J=7.6 Hz, 1H), 6.46 (t, 1H), 6.61 (t, 2H), 6.71-6.83 (m, 3H), 6.97 (t, 2H), 7.18 (d, J=7.2 Hz, 1H), 9.05 (s, 1H); ¹³C NMR (CDCl₃, 100 MHz) in ppm: δ 25.4, 34.8, 45.2, 60.8, 111.3, 116.7, 119.5, 122.8, 123.0, 125.6, 126.5, 127.3, 128.2, 128.5, 128.6, 131.6, 138.4, 139.2, 140.7, 150.7, 155.1, 185.4; RP-HPLC on a C18 Xbridge column, retention time 8.19 min, employing a linear gradient system of acetonitrile-water: 70%-100% B in A for 15 min. Where A is 0.1% AcOH in water and B is 0.1% AcOH in acetonitrile; ESI-MS (MW calcd. 453.6) m/z=454.1 (M+H)⁺.

### 16. Preparation of 3,3-Bis(5-tert-Butyl-2-Hydroxyphenyl)-6,7-Difluoroindolin-2-One (HY036)

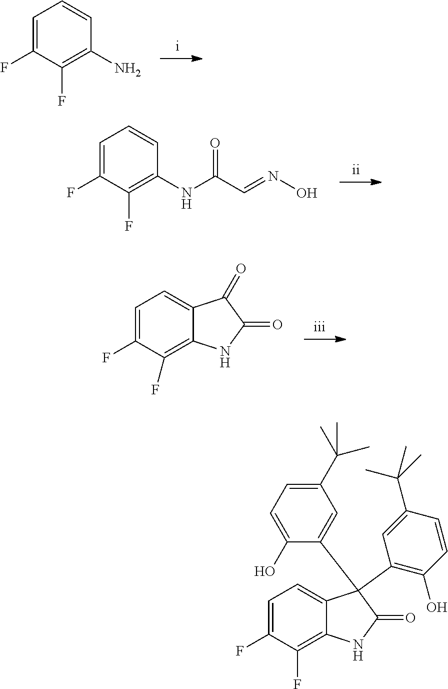 [SMILES: Nc1cccc(F)c1F ; O=C(/C=N/O)Nc1cccc(F)c1F ; O=C1Nc2c(ccc(F)c2F)C1=O ; CC(C)(C)c1ccc(O)c(C2(c3cc(C(C)(C)C)ccc3O)C(=O)Nc3c2ccc(F)c3F)c1]

In the above reaction, i. sodium sulfate (8.0 eqiv.), hydroxylamine hydrochloride (3.0 eqiv.), chloral hydrate (1.0 eqiv.), concentrated hydrochloric acid, water. ii. H₂SO₄. iii. 4-tert-butylphenol (1.0 eqiv.), p-TsOH (1.7 eqiv.), 1,2-dichloroethane.

### Step A Syntheses of N-(2,3-Difluorophenyl)-2-(Hydroxyimino)Acetamide^{[2]}

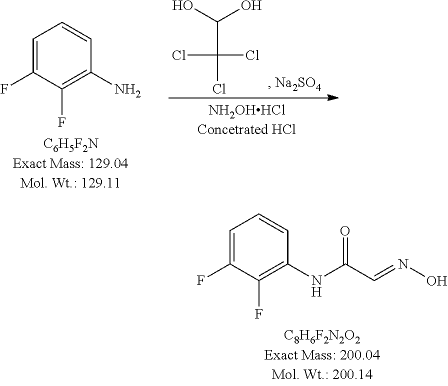 [SMILES: Nc1cccc(F)c1F ; OC(O)C(Cl)(Cl)Cl ; O=C(/C=N/O)Nc1cccc(F)c1F]

To a well stirred suspension of sodium sulfate (26.5 g, 186 mmol, 8 equivalents) in water (60 mL) at 60° C. were added in sequence hydroxylamine hydrochloride (4.83 g, 70 mmol, 3 equivalents), chloral hydrate (4.23 g, 25.6 mmol, 1.1 equivalents), 2,3-difluoroamine (2.5 g, 23.3 mmol, 1 equivalent) in water (42 mL) and finally concentrated hydrochloric acid (1.87 mL). The mixture temperature was risen to 100° C. After 1 h, the brown solution was left to cool to room temperature and kept stirring overnight. The solid present was filtered, washed with water, cyclohexane and dried at 60° C. Under vacuum for 6 h. Obtained 4.7 g as a beige solid used without further purification. ESI-MS (MW calcd. 200.14) m/z=199.74 (M−H)⁻.

### Step B Syntheses of 6,7-Difluoroindoline-2,3-Dione

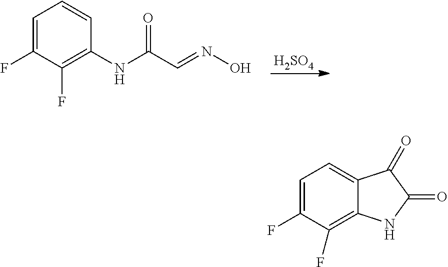

To well stirred sulphuric acid (12 mL) heated at 50° C. was added N-(2,3-difluorophenyl)-2-(hydroxyimino)acetamide (2.1 g, 10.5 mmol) in small portion over 20 min (exothermic up to 70° C.). After addition was completed, the temperature was risen to 80° C. And kept for 40 min after which the reaction was left cool to r. m. The brown mixture was slowly poured into ice and water, diluted with more water to yield brown mixture slurry. After extracted with ethyl acetate three times, the organic phase was merged. After all solvent was removed in vacuo, the crude product was purified by flash column chromatography on silica gel with the gradient of EtOAc in cyclohexane (10-30%) to afford 1.2 g (yield 57%) of yellow solid. ESI-MS (MW calcd. 183.11) m/z=181.70 (M−H)⁻.

### Step C Syntheses of 3,3-Bis(5-tert-Butyl-2-Hydroxyphenyl)-6,7-Difluoroindolin-2-One

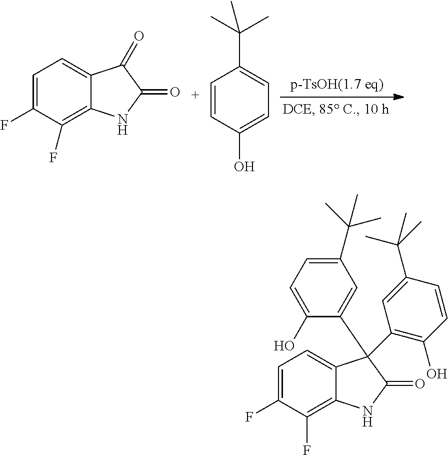

p-TsOH (3.4 mmol, 646 mg, 1.7 equivalents) was added to a solution of 4-tert-butylphenol (4.4 mmol, 594 mg, 2.2 equivalents) and 6,7-difluoroindoline-2,3-dione (2 mmol, 366 mg, 1.0 equivalent) in dry 1,2-dichloroethane. The stirred reaction mixture was heated for 10 h at 85° C. The residue obtained after the removal of the solvent under vacuum was purified by flash chromatography on silica gel with the gradient of EtOAc in cyclohexane (0-15%) to afford 502 mg (yield 54%, purity is 96.54%) of white solid. ¹H NMR (DMSO, 400 MHz) in ppm: δ 1.09 (s, 18H), 6.64-6.83 (m, 5H), 6.98-7.01 (m, 1H), 7.09 (s, 1H), 7.24 (s, 1H), 9.38 (s, 1H), 9.45 (s, 1H), 11.7 (s, 1H); RP-HPLC on a C18 Xbridge column, retention time 11.06 min, employing a linear gradient system of acetonitrile-water: 50%-100% B in A for 15 min. Where A is 0.1% AcOH in water and B is 0.1% AcOH in acetonitrile; ESI-MS (MW calcd. 465.53) m/z=464.03 (M−H)⁻.

### Example II

### Analysis of Diaryloxindole Compounds

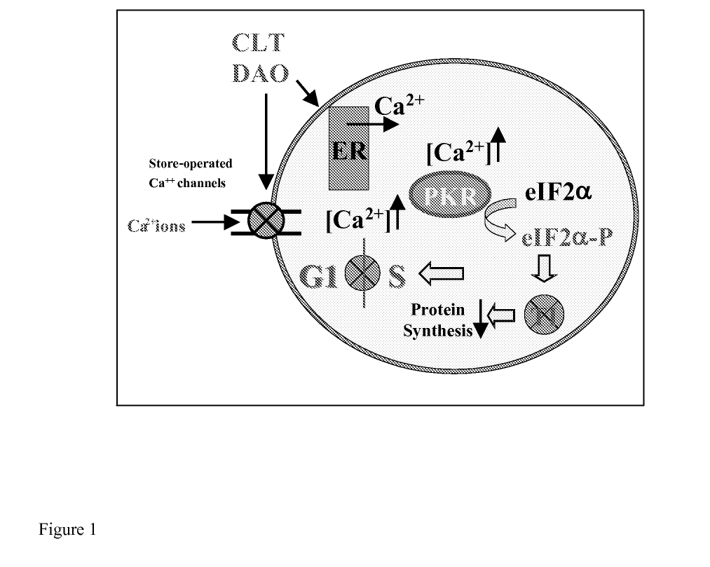

Without intending to be bound by theory, the mechanism of action of translation initiation inhibitors is set forth in FIG. 1, and includes the depletion (complete or partial depletion) of intracellular calcium (Ca²⁺) stores and phosphorylation of eIF2.

For assay development, a bi-directional plasmid was designed in which a common promoter/enhancer complex drives the transcription of firefly luciferase (F-luc) ORF fused to the 5′ untranslated region (UTR) of ATF-4, and of the renilla luciferase (R-luc) ORF fused to a 90-nucleotide 5′ UTR derived from the plasmid (FIG. 6A). Because the tetracycline-regulated transactivator ((tTA), required for driving transcription from this vector) is not normally expressed in the mammalian cells, stable KLN cancer cells were constructed that expressed tTA (KLN-tTA).

The KLN-tTA colonies that drove expression of reporter genes from pBISA-DL plasmid were selected by transient transfection and dual luciferase assay. One of these KLN-tTA cell lines was transfected with the pBISA-DL^{(ATF-4)} expression vector, stable colonies were selected by dual luciferase assay and expanded.

Plasmids and Ternary Complex Assay

(ATF-4 Assay). Briefly, bi-directional mammalian expression vector pBI (Clontech, CA) was modified to expand the multiple cloning sites (MCSs) and designated thereafter as pBISA. This vector contains seven copies of the tetracycline-regulated transactivator response element (TRE), which together act as core promoter/enhancer. The TRE is flanked on both sides by minimal human cytomegalovirus (CMV) minimal promoters allowing bi-directional transcription and two MCSs. Firefly and renilla luciferases were subcloned into MCS-I and MCS-II, respectively. This plasmid, designated pBISA-DL, transcribes two mRNAs that contain the 90 nucleotide plasmid derived 5′UTR (same sequence in both mRNAs), and the ORF encoding either firefly or renilla luciferase followed by a polyadenylation sequence as described in Ziegeler, G.; Ming, J.; Koseki, J. C.; Sevinc, S.; Chen, T.; Ergun, S.; Qin, X.; Aktas, B. H. J. Biol. Chem. 2010, 285, 15408 hereby incorporated by reference. This plasmid was further modified by inserting the 5′UTR of ATF-4 into MCS-I in front of the firefly luciferase mRNA. Transcription from this direction generates an mRNA that contains the firefly luciferase ORF preceded by a 5′UTR composed of 90 nucleotides derived from the plasmid and 267 nucleotides derived from the 5′UTR of ATF-4 mRNA. Transcription from the other direction generates an mRNA that contains the renilla luciferase ORF proceeded only by the 90-nucleotide plasmid-derived sequence in the 5′UTR. This expression plasmid is called pBISA-DL(ATF-4). In the dual luciferase assay, cells expressing firefly and renilla luciferases were lysed and the extracts assayed with a Dual-Glo Luciferase assay kit, per manufacturer's instruction (Promega Inc., Madison, Wis.). The data calculations were carried out as the ratio of firefly to renilla luciferase signal as described in Chen, T.; Ozel, D.; Qiao, Y.; Harbinski, F.; Chen, L.; Denoyelle, S.; He, X.; Zvereva, N.; Supko, J. G.; Chorev, M.; Halperin, J. A.; Aktas, B. H. Nat. Chem. Biol. 2011, 7, 610 hereby incorporated by reference.

In order to validate the compounds described herein as modifiers of the abundance of the eIF2•GTP•Met-tRNAᵢ ternary complex, the effects of the compounds on endogenous cellular markers of the ternary complex were determined. For example, reducing amount of the ternary complex increases translation of ATF-4, which results in elevated expression of CHOP mRNA and protein. Therefore, expression of endogenous CHOP mRNA and CHOP protein in KLN-tTA/pBISA-DL^{(ATF-4)} cells were utilized as secondary assays to validate the compounds as modifiers of the eIF2•GTP•Met-tRNAᵢ ternary complex.

Real Time PCR.

Total RNA was extracted with TaqMan Gene Expression Cells-to-Ct™ Kit (Applied Biosystems, Branchburg, N.J.) and DNAse I treated according to manufacturer's recommendations. 1-Step Real-time PCR was performed on a Bio-Rad iCycler IQ5 system by using B-R 1-Step SYBR Green qRT-PCR Kit (Quanta BioSciences, Gaithersburg, Md.) according to manufacturer's specifications. The thermal cycler conditions were as follows: 10 minutes at 50° C., hold for 5 minutes at 95° C., followed by 2-step PCR for 45 cycles of 95° C. for 15 seconds followed by 60° C. for 30 seconds. All PCRs were performed in triplicate in at least two independent PCR runs. Mean values of these repeated measurements were used for calculation. To calibrate the results, all the transcript quantities were normalized to 18S rRNA (18S ribosomal RNA-like mRNA in mouse). The following primers were used in real-time PCR reactions.

### Human CHOP

(SEQ ID NO: 1)
5' AGAACCAGGAAACGGAAACAGA 3'
(SEQ ID NO: 2)
5' TCTCCTTCATGCGCTGCTTT 3'

### Mouse CHOP

(SEQ ID NO: 3)
5' CATACACCACCACACCTGAAAG 3'
(SEQ ID NO: 4)
5' CCGTTTCCTAGTTCTTCCTTGC 3'

The effects of these compounds on the ternary complex assay and the expression of CHOP mRNA was assayed in adherent mouse (KLN) and human solid tumors cells (CRL-2351 and CRL-2813).

Cell Growth Assay.

Adherent mouse (KLN) and human solid tumor cells (CRL-2351, and CRL-2813) were plated in 96-well plates and maintained for 5 days in the presence of 0.54 to 6 μM of individual compound, and cell proliferation was measured by the sulforhodamine B (SRB) assay as described in ref. 11: briefly, cells were fixed in 10% cold trichloroacetic acid at 4° C. for 1 h, extensively washed with double-distilled H₂O and air-dried. Plates were then incubated with 0.4% SRB in 1% acetic acid for 1 h, washed with 1% acetic acid to remove the unbound dye, and air-dried. The bound dye was solubilized by addition of 10 mM Tris (pH 10), and the absorbance was determined in a Titertek Multiscan plate reader at 490 nm. The data calculations were carried out as described (the values for mean±SD of data from replicate wells are calculated): data are expressed in terms of % T/C [(OD of treated cells/OD of control cells)×100], as a measure of cell viability and survival in the presence of test materials. Calculations are also made for the concentration of test agents giving a T/C value of 50%, or 50% growth inhibition (IC₅₀). With the SRB assay, a measure is made of the cell population density at time 0 (the time at which drugs are added) from two extra reference plates of inoculated cells fixed with TCA just prior to drug addition to the test plates. Thus, we have three measurements: control optical density (C), test optical density (T), and optical density at time zero (T₀). The calculation is 100×[(T−T₀)/(C−T₀)]. If T is less than T₀, cell killing has occurred and can be calculated from 100×[(T−T₀)/T₀]. Thus, for each drug-cell line combination, a dose-response curve is generated and three levels of effect are calculated.

Table 1 sets forth biological data for diaryloxindole compounds.

| | IC₅₀ (μM) | | | CHOP PCR (μM) | | ATF-4 |
|---|---|---|---|---|---|---|
| Compounds | KLN | 2813 | 2351 | KLN | 2351 | Xmax @ 40 μM |
| 1181 | 13.5 ± 1.2 | 2.9 ± 0.3 | 3.8 ± 1 | | | 3.92 ± 0.17/40 |
| 1282 | >20 | 4.4 ± 1 | 5 ± 1.9 | 14.8 at | | Not Active |
| | | | | 20 uM | | |
| HY161 | >20 | 1.4 ± .09 | 7.2 ± 3 | | | 3.91 ± 0.65/40 |
| 1289 | 16 ± 2.2 | 2.5 ± 0.3 | 3.2 ± 1 | | | 2.87 ± 2.19/40 |
| HY069 | >20 | 2.9 ± 0.3 | 8.8 ± 0.9 | | | 10.7 ± 1 |
| | | | | | | At 40 uM |
| HY021 | 12 ± 0.7 | 10.2 ± 1.2 | 2.8 ± 0.4 | | | 5.2 ± 2.2/10 uM |
| | | | | | | 10.58/20 |
| | | | | | | 4.4/40 |
| HY050 | 15.2 ± 2 | 2.9 ± 0.3 | 2.6 ± 0.5 | | | 3.34 ± 0.07/20 |
| HY061 | 11 ± 0.4 | 2.8 ± 0.2 | 3 ± 0.6 | 4.23 at | | 2.9 ± 1.21/80 |
| | | | | 40 | | |
| | | | | 12.3 at | | |
| | | | | 80 uM | | |
| HY051 | >20 | 3.1 ± 0.2 | 5.3 ± 2.4 | 13.8 at | | 5.48 ± 0.30/20 uM |
| | | | | 20 uM | | |
| HY058 | 15.2 ± 1.1 | 1.3 ± 0.4 | 2.4 ± 0.1 | | | 3.35 ± 0.74/20 |
| HY169 | 11 ± 2.4 | 2.9 ± 0.1 | 2.7 ± 0.3 | | | 3.4 ± 0.1/at 20 uM |
| HY081 | >20 | 0.9 ± 0.1 | 3.9 ± 2 | 1.78 at | | 1.21 ± 0.16/40 |
| | | | | 40 uM | | |
| HY084 | 11.4 ± 0.2 | 1.6 ± 0.4 | 2.7 ± 0.2 | | | 8.59 ± 1.67/40 |
| | | | | | | 11.4 ± 0.4/80 uM |
| HY083 | 11.4 ± 2.2 | 1.0 ± 0.9 | 2.4 ± 1.6 | | | 12.94 ± 2.11/40 uM |
| HY032 | 16.1 ± 2.2 | 10.5 ± 1.9 | 4.8 ± 1.6 | | | 4.65 ± 0.58/40 uM |
| HY181 | >20 | 0.9 ± 0.1 | 3.9 ± 2 | 1.1 at | | 1.58 ± 0.07/80 uM |
| | | | | 80 uM | | |
| HY184 | >20 | 2 ± 0.9 | 2 ± 0.6 | | | 2.07 ± 1.60/80 uM |
| HY067 | 19.8 ± 1.3 | 0.24 | 2.1 ± 1.1 | | | 1.19 ± 0.19/80 uM |
| HY283 | >20 | 1.4 ± 0.4 | 1.5 ± 0.7 | | | 9.60 ± 0.16/40 uM |
| HY183 | >20 | 1.1 ± 0.3 | 2.9 ± 1.2 | 1.83 at | | 1.9 ± 0.03/80 uM |
| | | | | 80 | | |
| HY037 | >20 | 5.6 ± 0.8 | 3.6 ± 0.6 | | | 2.79 ± 0.9/80 uM |
| 1287 | >20 | 2.8 ± 0.3 | 3.5 ± 0.1 | | | |
| HY077 | 1.7 ± 0.9 | 1.2 ± 0.4 | 1.9 ± 1 | | | 4.65 ± 0.88/20 uM |
| HY087 | >20 | >20 | 2.7 ± 0.2 | | | 7.12 ± 1.30/20 uM |
| HY277 | 0.3 ± 0.1 | 0.9 ± 0.1 | 0.3 ± 0.11 | | | 14.27 ± 1.86/10 uM |
| HY096 | 0.8 ± 0.4 | 1.5 ± 0.6 | 0.6 ± 0.1 | 12.4 at | | 16.68 ± 3.24/20 uM |
| | | | | 20 uM | | |
| HY048 | 14.3 ± 1.6 | 3 ± 0.4 | 1.9 ± 0.5 | | | 5 ± 1.5/20 uM |
| HY033 | 15.2 ± 1.6 | 4.2 ± 0.2 | 6.8 ± 0.8 | | 7.8/20 uM | 6.14 ± 0.63/40 μM |
| | | | | | 18.5/40 uM | |
| HY177 | >20 | 12.5 ± 1 | 11.5 ± 1.5 | | | 1.34 ± 0.38/40 uM |
| HY092 | 0.2 ± 0.03 | 0.9 ± 0.01 | 0.34 ± 0.18 | | | 16.75 ± 2.18/2.5 uM |
| HY196 | 0.3 ± 0.1 | 1 ± 0.1 | 0.65 ± 0.1 | 13.1 at | | 16.79 ± 2.23/10 uM |
| | | | | 10 | | |
| HY091 | 1.4 ± 0.4 | 10.7 ± 0.8 | 1 ± 0.1 | | | 2.73 ± 0.65/10 uM |
| | | | | | | 5.3 ± 0.7/20 uM |
| HY093 | 4.1 ± 0.8 | 7.25 ± 1.6 | 3.6 ± 0.7 | | | 2.21 ± 0.75/20 uM |
| | | | | | | 9 ± 1.7/40 uM |
| HY098 | 0.5 ± 0.2 | 0.9 ± 0.2 | 0.3 ± 0.1 | | | 8.7 ± 1.2/10 uM |
| HY072 | 2.9 ± 0.4 | 3.35 ± 0.50 | 0.7 ± 0.1 | | | 4.15 ± 0.50/20 uM |
| HY063 | 7.4 ± 2.5 | 3.2 ± 0.2 | 1.6 ± 0.2 | | | 4.4 ± 1.5/40 uM |
| HY296 | 1.6 ± 0.5 | 0.72 ± 0.23 | 0.59 ± 0.26 | | | 14.26 ± .78/2.5 uM |
| HY191 | 3.5 ± 1.2 | 0.8 ± 0.1 | 0.65 ± 0.1 | 8.5 at | | 13.90 ± 0.97/5 uM |
| | | | | 5 uM | | |
| HY193 | 2.11 ± 1.1 | 1.47 ± 1.0 | 0.55 ± 0.17 | | | 12.1 ± 1.8 at 2.5 uM |
| | | | | | | 9.09/40 uM |
| HY198 | 0.30 ± 0.10 | 0.8 ± 0.1 | 0.2 ± 0.05 | 3.76 at | | 11.82 ± 0.11/2.5 uM |
| | | | | 20 | | |
| HY172 | 1.3 ± 0.3 | 11.6 ± 1.4 | 2.14 ± 1.28 | | | 5.91 ± 0.52/80 uM |
| SD167 | >20 | 2.4 ± 0.1 | 1.68 ± 1.26 | | | 4..98 ± 0.28/20 uM |
| HY304 | 17.5 ± 1.3 | 1.6 ± 0.2 | 2.2 ± 0.2 | 9.1 at | | 4.31 ± 0.10/40 uM |
| | | | | 40 | | |
| HY301 | 4.6 ± 1.2 | 1.5 ± 0.7 | 0.9 ± 0.2 | | | 15.08 ± 1.04/20 uM |
| HY080 | 2.9 ± 0.8 | 1.4 ± 0.5 | 0.75 ± 0.1 | | | 24.56 ± 3.37/20 uM |
| SD132 | >20 | 3.74 ± 0.28 | >20 | | | 8.8 ± 1.97 at 80 |
| | | | | | | uM |
| HY305 | 5 ± 3.5 | 2 ± 0.4 | 3.6 ± 0.7 | 10 at | | 3.61 ± 0.47/40 |
| | | | | 40 | | 31.89 ± 9.27/80 |
| HY306 | 2.3 ± 1.6 | 2.7 ± 0.8 | 2.3 ± 0.6 | 8.11 at | | 19.2 ± 11.2 at 40 uM |
| | | | | 20 uM | | |
| HY401 | 10.6 ± 0.9 | 1.5 ± 0.9 | 0.8 ± 0.1 | | | 11.4 ± 0.65/40 uM |
| HY413 | 0.85 ± 0.02 | 0.74 ± 0.01 | 0.2 ± 0.08 | | | 9.7 ± 3.8 at 10 uM |
| HY313 | 9.3 ± 3.2 | >20 | 0.6 ± 0.1 | 6.73 at | | NA |
| | | | | 10 uM | | |
| HY036 | 11.2 ± 3.2 | 2.7 ± 0.1 | 2.4 ± 0.6 | | | 4.45 ± 1.07/40 uM |
| HY190 | >20 | >20 | >20 | | | NA |
| HY055 | 15.6 ± 0.4 | 9.7 ± 2 | 9 ± 1.5 | | | 1.2 ± 0.6/80 uM |
| Sd029 | 6.8 ± 3.7 | 2.4 ± 0.3 | 3.35 ± 0.3 | | | 4 ± 0.1/40 |
| Sd028 | 1.2 ± 0.3 | 2.4 ± 0.4 | 1.4 ± 0.3 | | | 2.69 ± 0.36/10 |
| Sd049 | 1.2 ± 0.3 | 12.8 ± 0.8 | 0.9 ± 0.1 | | | 2.1 ± 0.65/20 |
| Sd123 | >20 | >20 | 17.5 ± 2 | | | 5.1 ± 1.65/80 |
| Sd177 | >20 | >20 | >20 | | | 1.65 ± 0.04 at 80 |
| Sd086 | 15.7 ± 1.1 | 2.75 ± 0.1 | 2.65 ± 0.1 | 4.09 | | 5.6 ± 0.5/40 |
| Sd139 | 4.3 ± 1 | 2.4 ± 0.9 | 2.65 ± 1.5 | 7.1 | | 6.3 ± 0.3/40 |
| Sd153 | 10.9 ± 1.3 | 11 ± 0.58 | 2.3 ± 0.3 | | | 3.55 ± at 40 |
| Sd140 | 12.3 ± 2 | 3 ± 0.9 | 0.95 ± 0.08 | | | 1.2 ± 0.46/40 |
| Sd184 | 14.3 ± 3 | 11.1 ± 1.1 | 2.9 ± 0.3 | | | 4 ± 0.36/40 |
| Sd185 | 2.75 ± 0.3 | 2.9 ± 0.3 | 4.5 ± 0.7 | | | 3.92 ± 0.16/40 |
| Sd202 | >20 | 12.2 ± 0.5 | 3.4 ± 0.6 | | | 4.12 ± 0.42/40 |
| Sd203 | 17 ± 3.5 | 14.6 ± 2.8 | 16.3 ± 1.6 | | | 1.51 ± 0.3/5 |
| Sd204 | 12 ± 1.2 | 2.6 ± 0.4 | 6.1 ± 1.7 | | | 4.5 ± 0.93/40 |
| Sd225 | 9.3 ± 2.7 | 1.9 ± 0.7 | 2.9 ± 0.27 | 3.49 | | 5.11 ± 0.48/40 |
| Sd163 | 15.7 ± 1.9 | 11 ± 1.3 | 4 ± 1 | | | 5.1 ± 0.1/80 |
| Sd096 | 17.3 ± 1.8 | 4 ± 0.28 | 2 ± 0.3 | | | 1.69 ± 0.6/40 |
| Sd154 | 2.2 ± 0.6 | 7.5 ± 1 | 4.7 ± 0.3 | 4.15 | | 7.15 ± 0.49/40 |
| Sd152 | >20 | >20 | >20 | | | 2.8/80 |
| Sd205 | 3.4 ± 1.2 | 2.7 ± 0.4 | 2.5 ± 0.6 | | | 5.2 ± 2.1/80 |
| Sd223 | 14.9 ± 3.8 | 2.8 ± 0.2 | 3.5 | 4.36 | | 7.2 ± 1.2/40 |
| Sd224 | 8 ± 1.7 | 1 ± 0.1 | 2.5 ± 0.9 | | | 2.56 ± 0.3/40 |
| Sd233 | 8 ± 3.3 | 0.95 ± 0.1 | 2.2 ± 1 | | | 1.2 ± 0.05/40 |
| Sd141 | 15.3 ± 1.9 | 10.7 ± 3.9 | >20 | 3.09 | | 2.9 ± 0.6/40 |
| Sd231 | 13.3 ± 2.4 | 2.5 ± 1.5 | 1.8 ± 0.4 | | | 15.6 ± 0.4/80 |
| Sd179 | 11.8 ± 1 | 13.4 ± 0.7 | 8.6 ± 1 | 20.8 | | 2.6 ± 0.14/80 |
| HY003 | >20 | 11.5 ± 1.8 | 6.4 ± 3 | | | 1.67/80 |
| HY016 | 17.3 ± 2.4 | 7.8 ± 0.66 | 7.3 ± 3.6 | 4.68 | | 8.5 ± 5.3/80 |
| HY017 | 4.3 ± 1 | 2.9 ± 0.1 | 2.9 ± 0.1 | | | 0.65 ± 0.7/40 |

Other embodiments will be evident to those of skill in the art. It should be understood that the foregoing description is provided for clarity only and is merely exemplary. The spirit and scope of the present invention are not limited to the above examples, but are encompassed by the following claims. All publications and patent applications cited above are incorporated by reference herein in their entirety for all purposes to the same extent as if each individual publication or patent application were specifically indicated to be so incorporated by reference.

1. A method of inhibiting translation initiation in cells or inhibiting abnormal proliferation of cells comprising contacting the cells with a compound of formula I 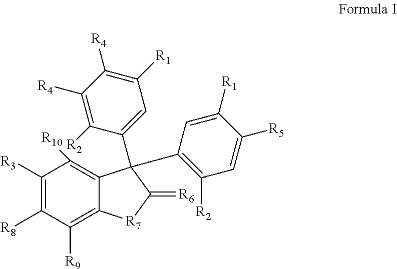
wherein
R₁ is the same or different and is selected from hydrogen, halogen, substituted or unsubstituted alkyl or heteroalkyl, substituted or unsubstituted alkenyl or heteroalkenyl, substituted or unsubstituted alkynyl or heteroalkynyl, cycloalkyl, —OR₁₁ where R₁₁ is substituted or unsubstituted alkyl or heteroalkyl, substituted or unsubstituted alkenyl or heteroalkenyl, or substituted or unsubstituted alkynyl or heteroalkynyl; exemplary R₁ is hydrogen, fluorine, chlorine, bromine, iodine, methyl, ethyl, propyl, n-propyl, isopropyl, butyl, n-butyl, isobutyl, tert-butyl, sec-butyl, pentyl, n-pentyl, cyclopentyl, hexyl, n-hexyl, cyclohexyl, heptyl, n-heptyl, cycloheptyl, methoxy, ethoxy, propoxy, butoxy, pentoxy, hexoxy, heptoxy or 2-oxy-3,4,5-trihydroxy-6-(hydroxymethyl)tetahydro-2H-pyran-2-yl;
R₂ is the same or different and is selected from hydrogen, hydroxyl, alkoxy, substituted alkoxy, methoxy, —O—CH₂—R₁₂, ethoxy, —O—CH₂—CH₂—R₂₁, —O—R₂₂, propargyloxy, 1H-1,2,3-triazol-1-yl, 4-(pyridin-2-ylmethyl)-1H-1,2,3-triazol-1-yl, 4-((5-(2-hydroxyethylamino)pyridin-2-yl)methyl)-1H-1,2,3-triazol-1-yl) wherein R₁₂ is 1H-1,2,3-triazol-4-yl, 1-(2-morpholinoethyl)-1H-1,2,3-triazol-4-yl, carboxymethylene, 2-acetamide, N-(2-hydroxyethyl)-2-acetamido, N-(2-(3,4,5-trihydroxy-6-(hydroxymethyl)tetrahydro-2H-pyran-2-yloxy)ethyl)acetamido, wherein R₂₁ is hydroxy, methoxy, amino, N-methylamino, N,N-dimethylamino, N,N-diethylamino, [N-methyl,N-ethyl]amino, bromo, acetoxy, N-morpholino, N-pyrrolidino, N-piperidino, N-piperazino, N-[4-methyl]-piperazino, ((3R,4S,5S,6R)-2-oxy-3,4,5-trihydroxy-6-(hydroxymethyl)tetrahydro-2H-pyran-2-yl) or 2-oxy-3,4,5-trihydroxy-6-(hydroxymethyl)tetrahydro-2H-pyran-2-yl), and wherein R₂₂ is substituted or unsubstituted alkyl or heteroalkyl, substituted or unsubstituted alkenyl or heteroalkenyl, or substituted or unsubstituted alkynyl or heteroalkynyl, heterocycloalkyl linked to O with an alkyl, alkenyl or alkynyl group, branched or unbranched; exemplary R₂ is hydrogen, hydroxy, methoxy, substituted methoxy, ethoxy, substituted ethoxy, N-(2-ethoxy)-morpholino, 2-(N,N-dimethylamino)ethoxy, N-(2-ethoxy)-piperazin-1-yl, (1-(N-2-ethyl-morpholino)-1H-1,2,3-triazol-5-yl)methoxy, 4-((5-((2-hydroxyethyl)amino)pyridin-2-yl)methyl)-1H-1,2,3-triazol-1-yl, 4-(pyridin-2-ylmethyl)-1H-1,2,3-triazol-1-yl, prop-2-ynyloxy, N-(3,4,5-trihydroxy-6-(hydroxymethyl)tetrahydro-2H-pyran-2-yl)oxy)ethyl)-2-oxyacetamido, 2-oxyacetic acid, 2-oxyethanol, 2-methoxyethoxy, 2-acetoxyethyl, 2-bromoethoxy, 2-acetoxyethoxy, ethyl 2-oxyacetate, 2-(N,N-dimethylamino)ethoxy, 2-(N,N-diethylamino)ethoxy, 2-(pyrrolidin-1-yl)ethoxy, 2-(piperidin-1-yl)ethoxy, 2-morpholinoethoxy, 2-(piperazin-1-yl)ethoxy, or 2-(4-methylpiperazin-1-yl)ethoxy;
R₃ is hydrogen, halogen, —NH₂, morpholino, morpholinoalkyl, morpholinomethyl, morpholinoethyl, pyridino, 6-morpholinopyridin-3-yl, —OR₁₂ where R₁₂ is substituted or unsubstituted alkyl or heteroalkyl, substituted or unsubstituted alkenyl or heteroalkenyl, or substituted or unsubstituted alkynyl, heteroalkynyl or R₃ is connected to R₁₀ wherein R₃—X—R₁₀ is S—C═N or N═C—S;
R₄ is the same or different and is selected from hydrogen, hydroxy, alkyl, methyl, ethyl, halogen, —N(CH₃)₂, alkoxy, heteroalkyl, or —OR₁₂ where R₁₂ is substituted or unsubstituted alkyl or heteroalkyl, substituted or unsubstituted alkenyl or heteroalkenyl, or substituted or unsubstituted alkynyl or heteroalkynyl;
R₅ is hydrogen or hydroxy;
R₆ is oxygen, sulfur, NH or CH₂;
R₇ is oxygen, SH, NH or CH₃;
R₈ is hydrogen, halogen, fluorine, chlorine, bromine, iodine, carboxylate, carboxylate alkyl ester, alkoxycarbonyl or methoxycarbonyl;
R₉ is hydrogen, halogen, fluorine, chlorine, bromine, iodine, carboxylate, carboxylate alkyl ester, alkoxycarbonyl or methoxycarbonyl; and
R₁₀ is hydrogen or R₁₀ is connected to R₃ wherein R₁₀—X—R₃ is S—C═N or N═C—S;

2. The method of claim 1 wherein the cells are cancer cells.

3. The method of claim 1 carried out in an individual by administration of the compound to the individual.

4. The method of claim 4 wherein the compound is administered by inhalation, transdermally, orally, rectally, transmucosally, intestinally, parenterally, intramuscularly, subcutaneously or intravenously.

5. A method of inhibiting translation initiation in cells or inhibiting abnormal proliferation of cells comprising contacting the cells with a compound of formula II 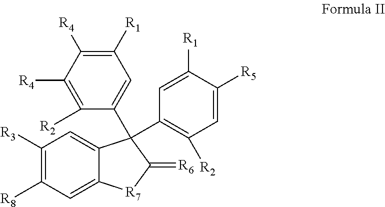
wherein R₁ is hydrogen, fluorine, chlorine, bromine, iodine, methyl, ethyl, propyl, n-propyl, isopropyl, butyl, n-butyl, isobutyl, tert-butyl, sec-butyl, pentyl, n-pentyl, cyclopentyl, hexyl, n-hexyl, cyclohexyl, heptyl, n-heptyl, cycloheptyl, methoxy, ethoxy. propoxy, butoxy, pentoxy, hexoxy, heptoxy or 2-oxy-3,4,5-trihydroxy-6-(hydroxymethyl)tetahydro-2H-pyran-2-yl;
R₂ is the same or different and is selected from hydrogen, hydroxyl, alkoxy, substituted alkoxy, methoxy, —O—CH₂—R₁₂, ethoxy, —O—CH₂—CH₂—R₂₁, propargyloxy, 1H-1,2,3-triazol-1-yl, 4-(pyridin-2-ylmethyl)-1H-1,2,3-triazol-1-yl, 4-((5-(2-hydroxyethylamino)pyridin-2-yl)methyl)-1H-1,2,3-triazol-1-yl) wherein R₁₂ is 1H-1,2,3-triazol-4-yl, 1-(2-morpholinoethyl)-1H-1,2,3-triazol-4-yl, carboxymethylene, 2-acetamide, N-(2-hydroxyethyl)-2-acetamido, N-(2-(3,4,5-trihydroxy-6-(hydroxymethyl)tetrahydro-2H-pyran-2-yloxy)ethyl)acetamido, wherein R₂₁ is hydroxy, methoxy, amino, N-methylamino, N,N-dimethylamino, N,N-diethylamino, [N-methyl,N-ethyl]amino, bromo, acetoxy, N-morpholino, N-pyrrolidino, N-piperidino, N-piperazino, N-[4-methyl]-piperazino, ((3R,4S,5S,6R)-2-oxy-3,4,5-trihydroxy-6-(hydroxymethyl)tetrahydro-2H-pyran-2-yl) or 2-oxy-3,4,5-trihydroxy-6-(hydroxymethyl)tetrahydro-2H-pyran-2-yl);
R₃ is hydrogen, fluorine, chlorine, bromine, iodine, trifluoroacetyl, 6-morpholinopyridin-3-yl, —NH₂, methoxy, ethoxy, propoxy, butoxy, pentoxy, hexoxy, or heptoxy;
R₄ is the same or different and is selected from hydrogen, hydroxyl, methyl, fluorine, chlorine, bromine, iodine, —N(CH₃)₂, methoxy, ethoxy, propoxy, butoxy, pentoxy, hexoxy, heptoxy, morpholinomethyl, 2-oxy-3,4,5-trihydroxy-6-(hydroxymethyl)tetrahydro-2H-pyran-2-yl, or N-(2-(3,4,5-trihydroxy-6-(hydroxymethyl)tetrahydro-2H-pyran-2-yloxy)ethyl)acetamido;
R₅ is hydrogen or hydroxyl;
R₆ is oxygen, sulfur, NH, or CH₂;
R₇ is O, SH, NH or CH₃; and
R₈ is hydrogen, halogen, fluorine, chlorine, bromine, iodine, carboxylate, carboxylate alkyl ester, alkoxycarbonyl or methoxycarbonyl.

6. The method of claim 5 wherein the cells are cancer cells.

7. The method of claim 5 carried out in an individual by administration of the compound to the individual.

8. The method of claim 7 wherein the compound is administered by inhalation, transdermally, orally, rectally, transmucosally, intestinally, parenterally, intramuscularly, subcutaneously or intravenously.

9. A method of inhibiting translation initiation in cells or inhibiting abnormal proliferation of cells comprising contacting the cells with a compound of formula III 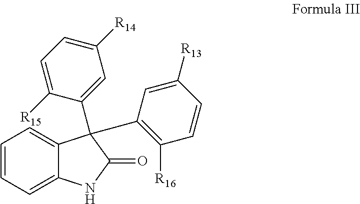
wherein R₁₃ is hydrogen, fluorine, chlorine, bromine, iodine, methyl, ethyl, propyl, n-propyl, isopropyl, butyl, n-butyl, isobutyl, tert-butyl, sec-butyl, pentyl, n-pentyl, cyclopentyl, hexyl, n-hexyl, cyclohexyl, heptyl, n-heptyl, cycloheptyl, methoxy, ethoxy. propoxy, butoxy, pentoxy, hexoxy, heptoxy or 2-oxy-3,4,5-trihydroxy-6-(hydroxymethyl)tetahydro-2H-pyran-2-yl.
R₁₄ is hydrogen, fluorine, chlorine, bromine, iodine, methyl, ethyl, propyl, n-propyl, isopropyl, butyl, n-butyl, isobutyl, tert-butyl, sec-butyl, pentyl, n-pentyl, cyclopentyl, hexyl, n-hexyl, cyclohexyl, heptyl, n-heptyl, cycloheptyl, methoxy, ethoxy. propoxy, butoxy, pentoxy, hexoxy, heptoxy or 2-oxy-3,4,5-trihydroxy-6-(hydroxymethyl)tetahydro-2H-pyran-2-yl.
R₁₅ is hydrogen, hydroxyl, alkoxy, substituted alkoxy, methoxy, —O—CH₂—R₁₂, ethoxy, —O—CH₂—CH₂—R₂₁, propargyloxy, 1H-1,2,3-triazol-1-yl, 4-(pyridin-2-ylmethyl)-1H-1,2,3-triazol-1-yl, 4-((5-(2-hydroxyethylamino)pyridin-2-yl)methyl)-1H-1,2,3-triazol-1-yl) wherein R₁₂ is 1H-1,2,3-triazol-4-yl, 1-(2-morpholinoethyl)-1H-1,2,3-triazol-4-yl, carboxymethylene, 2-acetamide, N-(2-hydroxyethyl)-2-acetamido, N-(2-(3,4,5-trihydroxy-6-(hydroxymethyl)tetrahydro-2H-pyran-2-yloxy)ethyl)acetamido, wherein R₂₁ is hydroxy, methoxy, amino, N-methylamino, N,N-dimethylamino, N,N-diethylamino, [N-methyl,N-ethyl]amino, bromo, acetoxy, N-morpholino, N-pyrrolidino, N-piperidino, N-piperazino, N-[4-methyl]-piperazino, ((3R,4S,5S,6R)-2-oxy-3,4,5-trihydroxy-6-(hydroxymethyl)tetrahydro-2H-pyran-2-yl) or 2-oxy-3,4,5-trihydroxy-6-(hydroxymethyl)tetrahydro-2H-pyran-2-yl); and
R₁₆ is hydrogen, hydroxyl, alkoxy, substituted alkoxy, methoxy, —O—CH₂—R₁₂, ethoxy, —O—CH₂—CH₂—R₂₁, propargyloxy, 1H-1,2,3-triazol-1-yl, 4-(pyridin-2-ylmethyl)-1H-1,2,3-triazol-1-yl, 4-((5-(2-hydroxyethylamino)pyridin-2-yl)methyl)-1H-1,2,3-triazol-1-yl) wherein R₁₂ is 1H-1,2,3-triazol-4-yl, 1-(2-morpholinoethyl)-1H-1,2,3-triazol-4-yl, carboxymethylene, 2-acetamide, N-(2-hydroxyethyl)-2-acetamido, N-(2-(3,4,5-trihydroxy-6-(hydroxymethyl)tetrahydro-2H-pyran-2-yloxy)ethyl)acetamido, wherein R₂₁ is hydroxy, methoxy, amino, N-methylamino, N,N-dimethylamino, N,N-diethylamino, [N-methyl,N-ethyl]amino, bromo, acetoxy, N-morpholino, N-pyrrolidino, N-piperidino, N-piperazino, N-[4-methyl]-piperazino, ((3R,4S,5S,6R)-2-oxy-3,4,5-trihydroxy-6-(hydroxymethyl)tetrahydro-2H-pyran-2-yl) or 2-oxy-3,4,5-trihydroxy-6-(hydroxymethyl)tetrahydro-2H-pyran-2-yl).

10. The method of claim 9 wherein the cells are cancer cells.

11. The method of claim 9 carried out in an individual by administration of the compound to the individual.

12. The method of claim 11 wherein the compound is administered by inhalation, transdermally, orally, rectally, transmucosally, intestinally, parenterally, intramuscularly, subcutaneously or intravenously.

13. A method of inhibiting translation initiation in cells or inhibiting abnormal proliferation of cells comprising contacting the cells with a compound of formula IV 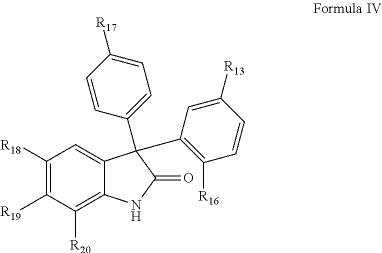
wherein R₁₃ is hydrogen, fluorine, chlorine, bromine, iodine, methyl, ethyl, propyl, n-propyl, isopropyl, butyl, n-butyl, isobutyl, tert-butyl, sec-butyl, pentyl, n-pentyl, cyclopentyl, hexyl, n-hexyl, cyclohexyl, heptyl, n-heptyl, cycloheptyl, methoxy, ethoxy. propoxy, butoxy, pentoxy, hexoxy, heptoxy or 2-oxy-3,4,5-trihydroxy-6-(hydroxymethyl)tetahydro-2H-pyran-2-yl;
R₁₆ is hydrogen, hydroxyl, alkoxy, substituted alkoxy, methoxy, —O—CH₂—R₁₂, ethoxy, —O—CH₂—CH₂—R₂₁, propargyloxy, 1H-1,2,3-triazol-1-yl, 4-(pyridin-2-ylmethyl)-1H-1,2,3-triazol-1-yl, 4-((5-(2-hydroxyethylamino)pyridin-2-yl)methyl)-1H-1,2,3-triazol-1-yl) wherein R₁₂ is 1H-1,2,3-triazol-4-yl, 1-(2-morpholinoethyl)-1H-1,2,3-triazol-4-yl, carboxymethylene, 2-acetamide, N-(2-hydroxyethyl)-2-acetamido, N-(2-(3,4,5-trihydroxy-6-(hydroxymethyl)tetrahydro-2H-pyran-2-yloxy)ethyl)acetamido, wherein R₂₁ is hydroxy, methoxy, amino, N-methylamino, N,N-dimethylamino, N,N-diethylamino, [N-methyl,N-ethyl]amino, bromo, acetoxy, N-morpholino, N-pyrrolidino, N-piperidino, N-piperazino, N-[4-methyl]-piperazino, ((3R,4S,5S,6R)-2-oxy-3,4,5-trihydroxy-6-(hydroxymethyl)tetrahydro-2H-pyran-2-yl) or 2-oxy-3,4,5-trihydroxy-6-(hydroxymethyl)tetrahydro-2H-pyran-2-yl);
R₁₇ is hydrogen, hydroxyl, methyl, fluorine, chlorine, bromine, iodine, —N(CH₃)₂, methoxy, ethoxy, propoxy, butoxy, pentoxy, hexoxy, heptoxy, morpholinomethyl, 2-oxy-3,4,5-trihydroxy-6-(hydroxymethyl)tetrahydro-2H-pyran-2-yl, or N-(2-(3,4,5-trihydroxy-6-(hydroxymethyl)tetrahydro-2H-pyran-2-yloxy)ethyl)acetamido;
R₁₈ is hydrogen, fluorine, chlorine, bromine, iodine, trifluoroacetyl, 6-morpholinopyridin-3-yl, —NH₂, methoxy, ethoxy, propoxy, butoxy, pentoxy, hexoxy, or heptoxy.
R₁₉ is hydrogen, halogen, fluorine, chlorine, bromine, iodine, carboxylate, carboxylate alkyl ester, alkoxycarbonyl or methoxycarbonyl; and
R₂₀ is hydrogen, halogen, fluorine, chlorine, bromine, iodine, carboxylate, carboxylate alkyl ester, alkoxycarbonyl or methoxycarbonyl.

14. The method of claim 13 wherein the cells are cancer cells.

15. The method of claim 13 carried out in an individual by administration of the compound to the individual.

16. The method of claim 15 wherein the compound is administered by inhalation, transdermally, orally, rectally, transmucosally, intestinally, parenterally, intramuscularly, subcutaneously or intravenously.

17. A method of inhibiting translation initiation in cells or inhibiting abnormal proliferation of cells comprising contacting the cells with a compound of formula V 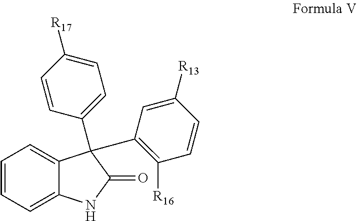
wherein R₁₃ is hydrogen, fluorine, chlorine, bromine, iodine, methyl, ethyl, propyl, n-propyl, isopropyl, butyl, n-butyl, isobutyl, tert-butyl, sec-butyl, pentyl, n-pentyl, cyclopentyl, hexyl, n-hexyl, cyclohexyl, heptyl, n-heptyl, cycloheptyl, methoxy, ethoxy. propoxy, butoxy, pentoxy, hexoxy, heptoxy or 2-oxy-3,4,5-trihydroxy-6-(hydroxymethyl)tetahydro-2H-pyran-2-yl;
R₁₆ is hydrogen, hydroxyl, alkoxy, substituted alkoxy, methoxy, —O—CH₂—R₁₂, ethoxy, —O—CH₂—CH₂—R₂₁, propargyloxy, 1H-1,2,3-triazol-1-yl, 4-(pyridin-2-ylmethyl)-1H-1,2,3-triazol-1-yl, 4-((5-(2-hydroxyethylamino)pyridin-2-yl)methyl)-1H-1,2,3-triazol-1-yl) wherein R₁₂ is 1H-1,2,3-triazol-4-yl, 1-(2-morpholinoethyl)-1H-1,2,3-triazol-4-yl, carboxymethylene, 2-acetamide, N-(2-hydroxyethyl)-2-acetamido, N-(2-(3,4,5-trihydroxy-6-(hydroxymethyl)tetrahydro-2H-pyran-2-yloxy)ethyl)acetamido, wherein R₂₁ is hydroxy, methoxy, amino, N-methylamino, N,N-dimethylamino, N,N-diethylamino, [N-methyl,N-ethyl]amino, bromo, acetoxy, N-morpholino, N-pyrrolidino, N-piperidino, N-piperazino, N-[4-methyl]-piperazino, ((3R,4S,5S,6R)-2-oxy-3,4,5-trihydroxy-6-(hydroxymethyl)tetrahydro-2H-pyran-2-yl) or 2-oxy-3,4,5-trihydroxy-6-(hydroxymethyl)tetrahydro-2H-pyran-2-yl); and
R₁₇ is hydrogen, hydroxyl, methyl, fluorine, chlorine, bromine, iodine, —N(CH₃)₂, methoxy, ethoxy, propoxy, butoxy, pentoxy, hexoxy, heptoxy, morpholinomethyl, 2-oxy-3,4,5-trihydroxy-6-(hydroxymethyl)tetrahydro-2H-pyran-2-yl, or N-(2-(3,4,5-trihydroxy-6-(hydroxymethyl)tetrahydro-2H-pyran-2-yloxy)ethyl)acetamido.

18. The method of claim 17 wherein the cells are cancer cells.

19. The method of claim 17 carried out in an individual by administration of the compound to the individual.

20. The method of claim 19 wherein the compound is administered by inhalation, transdermally, orally, rectally, transmucosally, intestinally, parenterally, intramuscularly, subcutaneously or intravenously.

21. A method of inhibiting translation initiation in cells or inhibiting abnormal proliferation of cells comprising contacting the cells with a compound of formula VI 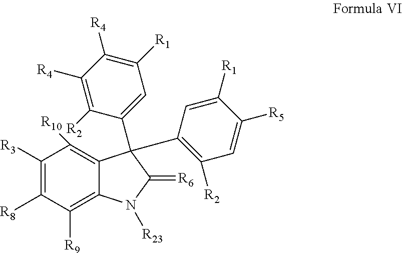
wherein R₁ is the same or different and is selected from hydrogen, halogen, substituted or unsubstituted alkyl or heteroalkyl, substituted or unsubstituted alkenyl or heteroalkenyl, substituted or unsubstituted alkynyl or heteroalkynyl, cycloalkyl, —OR₁₁ where R₁₁ is substituted or unsubstituted alkyl or heteroalkyl, substituted or unsubstituted alkenyl or heteroalkenyl, or substituted or unsubstituted alkynyl or heteroalkynyl; exemplary R₁ is hydrogen, fluorine, chlorine, bromine, iodine, methyl, ethyl, propyl, n-propyl, isopropyl, butyl, n-butyl, isobutyl, tert-butyl, sec-butyl, pentyl, n-pentyl, cyclopentyl, hexyl, n-hexyl, cyclohexyl, heptyl, n-heptyl, cycloheptyl, methoxy, ethoxy, propoxy, butoxy, pentoxy, hexoxy, heptoxy or 2-oxy-3,4,5-trihydroxy-6-(hydroxymethyl)tetahydro-2H-pyran-2-yl;
R₂ is the same or different and is selected from hydrogen, hydroxyl, alkoxy, substituted alkoxy, methoxy, —O—CH₂—R₁₂, ethoxy, —O—CH₂—CH₂—R₂₁, —O—R₂₂, propargyloxy, 1H-1,2,3-triazol-1-yl, 4-(pyridin-2-ylmethyl)-1H-1,2,3-triazol-1-yl, 4-((5-(2-hydroxyethylamino)pyridin-2-yl)methyl)-1H-1,2,3-triazol-1-yl) wherein R₁₂ is 1H-1,2,3-triazol-4-yl, 1-(2-morpholinoethyl)-1H-1,2,3-triazol-4-yl, carboxymethylene, 2-acetamide, N-(2-hydroxyethyl)-2-acetamido, N-(2-(3,4,5-trihydroxy-6-(hydroxymethyl)tetrahydro-2H-pyran-2-yloxy)ethyl)acetamido, wherein R₂₁ is hydroxy, methoxy, amino, N-methylamino, N,N-dimethylamino, N,N-diethylamino, [N-methyl,N-ethyl]amino, bromo, acetoxy, N-morpholino, N-pyrrolidino, N-piperidino, N-piperazino, N-[4-methyl]-piperazino, ((3R,4S,5S,6R)-2-oxy-3,4,5-trihydroxy-6-(hydroxymethyl)tetrahydro-2H-pyran-2-yl) or 2-oxy-3,4,5-trihydroxy-6-(hydroxymethyl)tetrahydro-2H-pyran-2-yl), and wherein R₂₂ is substituted or unsubstituted alkyl or heteroalkyl, substituted or unsubstituted alkenyl or heteroalkenyl, or substituted or unsubstituted alkynyl or heteroalkynyl, heterocycloalkyl linked to O with an alkyl, alkenyl or alkynyl group, branched or unbranched;
R₃ is hydrogen, halogen, —NH₂, morpholino, morpholinoalkyl, morpholinomethyl, morpholinoethyl, pyridino, 6-morpholinopyridin-3-yl, —OR₁₂ where R₁₂ is substituted or unsubstituted alkyl or heteroalkyl, substituted or unsubstituted alkenyl or heteroalkenyl, or substituted or unsubstituted alkynyl, heteroalkynyl or R₃ is connected to R₁₀ wherein R₃—X—R₁₀ is S—C═N or N═C—S, such as thiazolo, exemplary R₃ is hydrogen, fluorine, chlorine, bromine, iodine, trifluoroacetyl, 6-morpholinopyridin-3-yl, —NH₂, methoxy, ethoxy, propoxy, butoxy, pentoxy, hexoxy, or heptoxy;
R₄ is the same or different and is selected from hydrogen, hydroxy, alkyl, methyl, ethyl, halogen, —N(CH₃)₂, alkoxy, heteroalkyl, or —OR₁₂ where R₁₂ is substituted or unsubstituted alkyl or heteroalkyl, substituted or unsubstituted alkenyl or heteroalkenyl, or substituted or unsubstituted alkynyl or heteroalkynyl, exemplary R₄ is hydrogen, hydroxyl, methyl, fluorine, chlorine, bromine, iodine, —N(CH₃)₂, methoxy, ethoxy, propoxy, butoxy, pentoxy, hexoxy, heptoxy, morpholinomethyl, 2-oxy-3,4,5-trihydroxy-6-(hydroxymethyl)tetrahydro-2H-pyran-2-yl, or N-(2-(3,4,5-trihydroxy-6-(hydroxymethyl)tetrahydro-2H-pyran-2-yloxy)ethyl)acetamido;
R₅ is hydrogen or hydroxy;
R₆ is oxygen, sulfur, NH or CH₂;
R₈ is hydrogen, halogen, fluorine, chlorine, bromine, iodine, carboxylate, carboxylate alkyl ester, alkoxycarbonyl or methoxycarbonyl;
R₉ is hydrogen, halogen, fluorine, chlorine, bromine, iodine, carboxylate, carboxylate alkyl ester, alkoxycarbonyl or methoxycarbonyl;
R₁₀ is hydrogen or R₁₀ is connected to R₃ wherein R₁₀—X—R₃ is S—C═N or N═C—S; and
R₂₃ is hydrogen, 2-bromoethyl, 2(N,N-dimethylamino)ethyl, 2(N,N-diethylamino)ethyl, 2-(pyrrolidin-1-yl)ethyl, 2-morpholinoethyl, 2-(piperazin-1-yl)ethyl or 2-(4-methylpiperazin-1-yl)ethyl.

22. The method of claim 21 wherein the cells are cancer cells.

23. The method of claim 21 carried out in an individual by administration of the compound to the individual.

24. The method of claim 23 wherein the compound is administered by inhalation, transdermally, orally, rectally, transmucosally, intestinally, parenterally, intramuscularly, subcutaneously or intravenously.

25. A method of inhibiting translation initiation in cells or inhibiting abnormal proliferation of cells comprising contacting the cells with a compound of formula VII 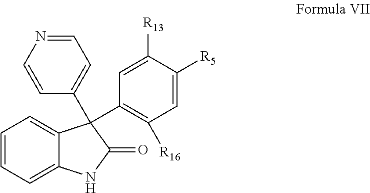
wherein R₁₃ is hydrogen, fluorine, chlorine, bromine, iodine, methyl, ethyl, propyl, n-propyl, isopropyl, butyl, n-butyl, isobutyl, tert-butyl, sec-butyl, pentyl, n-pentyl, cyclopentyl, hexyl, n-hexyl, cyclohexyl, heptyl, n-heptyl, cycloheptyl, methoxy, ethoxy. propoxy, butoxy, pentoxy, hexoxy, heptoxy or 2-oxy-3,4,5-trihydroxy-6-(hydroxymethyl)tetahydro-2H-pyran-2-yl.
R₁₆ is hydrogen, hydroxyl, alkoxy, substituted alkoxy, methoxy, —O—CH₂—R₁₂, ethoxy, —O—CH₂—CH₂—R₂₁, propargyloxy, 1H-1,2,3-triazol-1-yl, 4-(pyridin-2-ylmethyl)-1H-1,2,3-triazol-1-yl, 4-((5-(2-hydroxyethylamino)pyridin-2-yl)methyl)-1H-1,2,3-triazol-1-yl) wherein R₁₂ is 1H-1,2,3-triazol-4-yl, 1-(2-morpholinoethyl)-1H-1,2,3-triazol-4-yl, carboxymethylene, 2-acetamide, N-(2-hydroxyethyl)-2-acetamido, N-(2-(3,4,5-trihydroxy-6-(hydroxymethyl)tetrahydro-2H-pyran-2-yloxy)ethyl)acetamido, wherein R₂₁ is hydroxy, methoxy, amino, N-methylamino, N,N-dimethylamino, N,N-diethylamino, [N-methyl,N-ethyl]amino, bromo, acetoxy, N-morpholino, N-pyrrolidino, N-piperidino, N-piperazino, N-[4-methyl]-piperazino, ((3R,4S,5S,6R)-2-oxy-3,4,5-trihydroxy-6-(hydroxymethyl)tetrahydro-2H-pyran-2-yl) or 2-oxy-3,4,5-trihydroxy-6-(hydroxymethyl)tetrahydro-2H-pyran-2-yl); and
R₅ is hydrogen or hydroxyl.

26. The method of claim 25 wherein the cells are cancer cells.

27. The method of claim 25 carried out in an individual by administration of the compound to the individual.

28. The method of claim 27 wherein the compound is administered by inhalation, transdermally, orally, rectally, transmucosally, intestinally, parenterally, intramuscularly, subcutaneously or intravenously.

29. A method of inhibiting translation initiation in cells or inhibiting abnormal proliferation of cells comprising contacting the cells with a compound of formula VIII 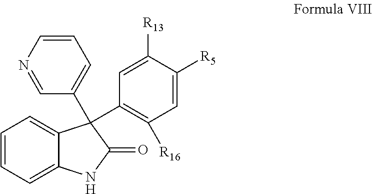
wherein R₁₃ is hydrogen, fluorine, chlorine, bromine, iodine, methyl, ethyl, propyl, n-propyl, isopropyl, butyl, n-butyl, isobutyl, tert-butyl, sec-butyl, pentyl, n-pentyl, cyclopentyl, hexyl, n-hexyl, cyclohexyl, heptyl, n-heptyl, cycloheptyl, methoxy, ethoxy. propoxy, butoxy, pentoxy, hexoxy, heptoxy or 2-oxy-3,4,5-trihydroxy-6-(hydroxymethyl)tetahydro-2H-pyran-2-yl.
R₁₆ is hydrogen, hydroxyl, alkoxy, substituted alkoxy, methoxy, —O—CH₂—R₁₂, ethoxy, —O—CH₂—CH₂—R₂₁, propargyloxy, 1H-1,2,3-triazol-1-yl, 4-(pyridin-2-ylmethyl)-1H-1,2,3-triazol-1-yl, 4-((5-(2-hydroxyethylamino)pyridin-2-yl)methyl)-1H-1,2,3-triazol-1-yl) wherein R₁₂ is 1H-1,2,3-triazol-4-yl, 1-(2-morpholinoethyl)-1H-1,2,3-triazol-4-yl, carboxymethylene, 2-acetamide, N-(2-hydroxyethyl)-2-acetamido, N-(2-(3,4,5-trihydroxy-6-(hydroxymethyl)tetrahydro-2H-pyran-2-yloxy)ethyl)acetamido, wherein R₂₁ is hydroxy, methoxy, amino, N-methylamino, N,N-dimethylamino, N,N-diethylamino, [N-methyl,N-ethyl]amino, bromo, acetoxy, N-morpholino, N-pyrrolidino, N-piperidino, N-piperazino, N-[4-methyl]-piperazino, ((3R,4S,5S,6R)-2-oxy-3,4,5-trihydroxy-6-(hydroxymethyl)tetrahydro-2H-pyran-2-yl) or 2-oxy-3,4,5-trihydroxy-6-(hydroxymethyl)tetrahydro-2H-pyran-2-yl); and
R₅ is hydrogen or hydroxyl.

30. The method of claim 29 wherein the cells are cancer cells.

31. The method of claim 29 carried out in an individual by administration of the compound to the individual.

32. The method of claim 31 wherein the compound is administered by inhalation, transdermally, orally, rectally, transmucosally, intestinally, parenterally, intramuscularly, subcutaneously or intravenously.


SEQUENCE LISTING (4 sequences)

SEQ ID NO: 1 - DNA, 22 bp, Homo sapiens
agaaccaggaaacggaaacaga

SEQ ID NO: 2 - DNA, 20 bp, Homo sapiens
tctccttcatgcgctgcttt

SEQ ID NO: 3 - DNA, 22 bp, Mus musculus
catacaccaccacacctgaaag

SEQ ID NO: 4 - DNA, 22 bp, Mus musculus
ccgtttcctagttcttccttgc